(19) United States
(12) Patent Application Publication
(10) Pub. No.: US 2012/0171195 A1
(43) Pub. Date: Jul. 5, 2012

(54) ANTI-HLA-E ANTIBODIES, THERAPEUTIC IMMUNOMODULATORY ANTIBODIES TO HUMAN HLA-E HEAVY CHAIN, USEFUL AS IVIG MIMETICS AND METHODS OF THEIR USE

(76) Inventors: Mepur H. Ravindranath, Los Angeles, CA (US); Paul I. Terasaki, Los Angeles, CA (US)

(21) Appl. No.: 12/983,856

(22) Filed: Jan. 3, 2011

(51) Int. Cl. A61K 39/395; A61P 31/04; A61P 31/12; A61P 3/10; A61P 11/00; A61P 29/00; A61P 27/02; A61P 27/16; A61P 7/00; A61P 37/02

(52) U.S. Cl. 424/133.1; 424/173.1; 424/142.1

(57) ABSTRACT

Provided herein are compositions comprising antibodies immunoreactive to human leukocyte antigen E (HLA-E) and HLA Ia alleles, methods of their use, for example, as therapeutic IVIg mimetics, methods of their preparation and methods of their immunomodulatory benefits and applications. In particular embodiments provided herein are compositions and methods for treating an inflammatory condition or graft rejection.

### FIELD OF THE INVENTION

Provided herein are IVIg mimetics useful for the prevention, treatment, therapy and/or amelioration of inflammation induced diseases and allograft rejection. In certain embodiments, provided herein are compositions comprising chimeric, humanized or human antibodies immunoreactive to HLA-E but not to other class Ib Human Leukocyte Antigens, namely, HLA-F or HLA-G. In particular embodiments provided herein, are compositions and methods for treating or ameliorating one or more inflammatory diseases and/or graft rejection using a composition comprising chimeric, humanized or human antibodies that are immunoreactive to HLA-E and also to HLA class la antigens, namely, FILA-A, HLA-B and HLA-Cw alleles.

### BACKGROUND

Intravenous immune globulin (IVIg) is a blood product administered intravenously. It contains the pooled IgG (immunoglobulin G) extracted from the plasma (without any other proteins) from over 1,000 to 60,000 blood donors. IVIg contains a high percentage of native human monomeric IgG with very low IgA content. IVIg's effects last between 2 weeks to 3 months.

When administered intravenously, IVIg has been shown to ameliorate several disease conditions. Therefore, the United States Food and Drug Administration has approved the use of IVIg for (1) Kawasaki disease; (2) immune-mediated thrombocytopenia; (3) primary immunodeficiencies; (4) hematopoietic stem cell transplantation (for those older than 20 yrs); (5) chronic B-cell lymphocytic leukemia; and (6) pediatric HIV type 1 infection. In 2004, the FDA approved the Cedars-Sinai IVIg Protocol for kidney transplant recipients so that such recipients could accept a living donor kidney from any healthy donor, regardless of blood type (ABO incompatible) or tissue match.

In addition, several other inflammatory diseases are also treated with IVIg, listed below:

1. Solid organ transplantation

2. Hematological Diseases
a. Aplastic anemia
b. Pure red cell aplasia
c. Diamond-Blackfan anemia
d. Autoimmune hemolytic anemia
e. Hemolytic disease of the newborn
f. Acquired factor inhibitors
g. Acquired von Willebrand disease
h. Immune-mediated neutropenia
i. Refractoriness to platelet transfusion
j. Neonatal alloimmune/ne thrombocytopenia
k. Post transfusion purpura
l. Thrombotic thrombocytopenia purpura/hemolytic uremic syndrome
m. Hemolytic transfusion reaction
n. Hemophagocytic syndrome
o. Thrombocytopenia
p. Acute lymphoblastic leukemia
q. Multiple myeloma
r. Human T-cell lymphotrophic virus-1-myelopathy

3. Nephropathy
a. Nephritic syndrome
b. Membranous nephropathy
c. Nephrotic syndrome
d. Acute renal failure

4. Neuropathy
a. Epilepsy
b. Chronic inflammatory demyelinating polyneuropathy and Guillain-Barre syndrome
c. Myasthenia gravis
d. Lambert-Eaton myasthenic syndrome
e. Multifocal motor neuropathy
f. Multiple sclerosis
g. Wegener granulomatosis
h. Amyotrophic lateral sclerosis
i. Lower motor neuron syndrome
j. Acute disseminated encephalomyelitis
k. Paraneoplastic cerebellar degeneration
l. Paraproteinemic neuropathy
m. Polyneuropathy,
n. Progressive lumbosacral plexopathy

5. Infection
a. HIV infection
b. Lyme radiculoneuritis
c. Endotoxemia of Pregnancy
d. Parvovirus infection
e. Streptococcal toxic shock syndrome

6. Autoimmune Diseases
a. Rheumatoid arthritis
b. Systemic lupus erythematosus
c. Systemic vasculitis
d. Dermatomyositis, polymyositis
e. Inclusion-body myositis
f. Autoimmune blistering dermatosis

7. Cardiomyopathy
a. Acute cardiomyopathy

8. Eye and Ear diseases
a. Euthyroid ophthalmopathy
b. Uveitis
c. Recurrent otitis media

9. Lung diseases
a. Asthma
b. Cystic fibrosis

10. Other disease conditions
a. Recurrent pregnancy loss.
b. Behcet syndrome
c. Chronic fatigue syndrome
d. Congenital heart block
e. Diabetes mellitus
f. Acute idiopathic dysautonomia
g. Opsoclonus-myoclonus
h. Rasmussen syndrome
i. Reiter syndrome
j. Vogt-Koyanagi-Harada syndrome trauma,
k. burns

IVIg is also presently used as a therapeutic immunomodulatory agent. For instance, IVIg is administered at a high dose (generally 1-2 grams IVIg per kg body weight) to decrease the severity of the immune response in patients with autoimmune diseases. Previous studies have shown that IgG antibodies in IVIg have immunosuppressive capabilities. It remains unclear from these studies, however, how these IgG antibodies act as immunomodulatory agents in the context of IVIg and whether these immunomodulatory effects are due to all IgGs or specific IgGs within IVIg. To date, the major component of IVIg that may be responsible for its immunomodulatory function has not been identified. Preparations of IVIg require labor-intensive and cost-intensive processes. See, e.g., access-medical.com/alpha-trax/Download/IGIV-ALPHA.ppt. It is well known that commercial preparations of IVIg vary in composition. See Table 1. A preparation of IVIg typically comprises pooled IgG from over a thousand blood donors. Reports in 2009 estimate that the utilization of IVIg (approx. $60/gm) regularly exceeds $10,000 per treatment course.

TABLE 1
Summary of Characteristics of Different
Commercial Preparations of IVIg

| Characteristics | Alpha | Baxter | Bayer | Centeon | Novartis |
|---|---|---|---|---|---|
| Donor Pool | 10,000 | 8,000 | 2,000 | 1,000 | 16,000 |
| (min) | | | | | |
| IgG (%) | >99 | >90 | >98 | >99 | >96 |
| IgG | All | Low IgG4 | All | Lowe IgG4 | All |
| IgA (mg/ml | 22 | <3.7 | 270 | 25 | 720 |

The lack of uniformity in commercial preparations of IVIg can lead to varying side effects among the different commercial preparations. Common adverse side effects include chills, headache, fever, nausea/vomiting, back pain, hypotension, joint pain and allergic responses. Serious adverse side effects include anaphylactic shock, renal insufficiency, Steven-Johnson syndrome, aseptic meningitis, thromboembolic events, thrombosis, cytopenia, hemolysis, stroke, seizure, loss of consciousness, acute respiratory distress syndrome, pulmonary edema, acute bronchospasm, transfusion associated lung injury, aseptic meningitis, delayed hemolytic reaction, acute myocardial infarction and even acute renal failure. Twenty-nine cases of thrombotic complications associated with the use of IVIg have been reported and include acute myocardial infarction, cerebral infarction, pulmonary embolism, deep venous thrombosis, hepatic veno-occlusive disease, and spinal cord ischemia. Specific adverse side effects were attributed to differences in osmolality, pH, and sugar and sodium content of IVIg products. Due to the varying side effects in the different IVIg commercial preparations, the FDA has allowed only certain IVIg preparations for the treatment of particular diseases. See Table 2.

TABLE 2
Summary of FDA Approved Uses of Different
Commercial Preparations of IVIg

| | Commercial IVIg Prep Approved by FDA | | | | |
|---|---|---|---|---|---|
| | for Treatment | | | | |
| Diseases | Alpha | Baxter | Bayer | Centeon | Novartis |
| Primary Immune | Yes | Yes | Yes | Yes | Yes |
| Deficiencies (PID) | | | | | |
| Idiopathic | Yes | Yes | Yes | No | Yes |
| Thrombocytopenic | | | | | |
| Purpura (ITP) | | | | | |
| Chronic | No | Yes | No | No | No |
| Lymphocytic | | | | | |
| Leukemia (CLL) | | | | | |
| Kawasaki Disease | Yes | Yes | No | No | No |
| Bone Marrow | No | No | Yes | No | No |
| Transplantation | | | | | |
| (BMT) | | | | | |
| Pediatric HIV | No | No | Yes | No | No |
| Infection | | | | | |

There is a need for a cost-effective IVIg substitute comprising a uniform composition that retains the therapeutic and/or prophylactic effects of IVIg while minimizing Wig related side effects.

### SUMMARY

Provided herein are IVIg mimetics useful for the prevention, treatment, therapy and/or amelioration of inflammation induced diseases and allograft rejection.

While not intending to be bound by any particular theory of operation, certain aspects provided herein are based, at least in part, on the identification of a potent immunoreactivity to HLA-E in commercial preparations of IVIg. The immunoreactivity increased with dilutions of Wig from 1/2 to 1/32 as shown in FIGS. 1A and 1B. The observation suggests that IVIg comprises aggregates of IgGs with immunoreactivity to HLA-E. These findings indicate that IgG with immunoreactivity to HLA-E is a substantial component of IVIg.

Further, while not intending to be bound by any particular theory of operation, certain aspects provided herein are based, at least in part, on the identification of immunoreactivity to free and β2-microglobulin-associated heavy chains of HLA-Ia accompanying the immunoreactivity of IVIg to HLA-E (FIGS. 2A and 2B). Both HLA-E and HLA-Ia immunoreactivity of IVIg was lost after adsorbing IVIg to gel conjugated only to HLA-E, indicating that the immunoreactivity to HLA-Ia is due to anti-HLA-E immunoreactivity in IVIg (FIGS. 3A and 3B). In particular, it has been observed that IVIg reacted to free and β2-microglobulin-associated heavy chains of several alleles of HLA-A, HLA-B and HLA-Cw, a feature characteristic of anti-FILA-E monoclonal antibodies (MAb-1 and MAb-2) that specifically react to HLA-E, but not to HLA-F or HLA-G among the non-classical HLA Ib molecules. These monoclonal antibodies were immunoreactive to free and β2-microglobulin-associated heavy chains of several HLA-Ia antigens (HLA-A alleles, HLA-B alleles and HLA-Cw alleles) (FIG. 4). Moreover, the HLA-E peptide sequences that were used to block the binding of anti-HLA-E antibodies to HLA-E also blocked the binding of the anti-HLA-E antibodies to HLA Ia alleles. See Ravindranath et al., 2010, Mol. Immunol. 47: 1121-1131; Ravindranath, et al., 2011, Mol. Immunol. 48: 423-430. Anti-HLA-E antibodies are also found in normal, non-alloimmunized, healthy males and HLA-Ia reactivity of anti-HLA-E IgG antibodies in the sera of these healthy individuals are also observed. Ravindranath et al., 2010, J. Immunol. 185: 1935-1948. IVIg's immunoreactivity to HLA-Ia, which is attributed to anti-HLA-E activity of IVIg, is identified to be strong and potent. These findings indicate that the anti-HLA-Ia reactivity of IVIg is associated with the anti-HLA-E IgG immunoreactivity of IVIg.

Further, while not intending to be bound by any particular theory of operation, certain aspects provided herein are based, at least in part, on the identification of T-cell suppressive immunomodulatory activity of human IVIg. This activity (FIGS. 5, 6, 9A-C, 10, 12 and 14) has been identified to be similar to the T-cell suppressive activity of different anti-HLA-E monoclonal antibodies (FIGS. 5, 7, 8, 9E-G, 11, 13 and 14).

Provided herein, in certain aspects, are chimeric, humanized or human anti-HLA-E antibodies immunoreactive to the heavy chain polypeptide of HLA-E and not immunoreactive to the heavy chain polypeptide of HLA-F or HLA-G.

Further provided herein, in certain aspects, are pharmaceutical compositions that can provide cost effective substitutes for IVIg. In certain embodiments, the pharmaceutical compositions are uniform in composition and can minimize the side effects often associated with the varying commercial preparations of IVIg. Certain pharmaceutical compositions provided herein comprise antibodies in a pharmaceutically acceptable carrier, wherein said antibodies are chimeric, humanized or human anti-HLA-E antibodies immunoreactive to HLA-E and not immunoreactive to HLA-F or HLA-G. In some embodiments, said anti-HLA-E antibodies are purified antibodies immunoreactive to the heavy chain polypeptide of HLA-E and not immunoreactive to the heavy chain polypeptide of HLA-F or HLA-G and not immunoreactive to β2-microglobulin.

In some embodiments, the anti-HLA-E antibodies are purified monoclonal antibodies. purified polyclonal antibodies, recombinantly produced antibodies, Fab fragments, F(ab′) fragments, or epitope-binding fragments. In particular embodiments, the anti-HLA-E antibodies are purified monoclonal antibodies. In particular embodiments, the anti-HLA-E antibodies are purified polyclonal antibodies. In other embodiments, the anti-HLA class-E antibodies are Fab fragments.

In some embodiments. the anti-HLA-E antibodies are IgG antibodies. In particular embodiments, the anti-HLA-E antibodies are IgG1 antibodies.

In some embodiments of the pharmaceutical compositions provided herein, the composition is suitable for intramuscular administration, intradermal administration, intraperitoneal administration, intravenous administration, subcutaneous administration, or any combination thereof. In some embodiments, the pharmaceutical composition is suitable for subcutaneous administration. In some embodiments, the composition is suitable for intravenous administration. In some embodiments, the composition is suitable for intramuscular administration.

In some embodiments, said anti-HLA-E antibodies are also immunoreactive to heavy chains of HLA-E and of one or more of HLA-A, HLA-B and HLA-Cw. In some embodiments, said heavy chains are free heavy chains, not associated with β2-microglobulin. In some embodiments, said heavy chains are associated with β2-microglobulin. In specific embodiments, said anti-HLA-E antibodies are also immunoreactive to heavy chains of HLA-E and of one or more of HLA-A, HLA-B and HLA-Cw present in the circulation or blood (plasma or serum), synovial fluid, seminal fluid or in any other body fluid, wherein the anti-HLA-E antibodies are capable of clearing and/or neutralizing soluble HLA-E and soluble HLA-A, HLA-B and HLA-Cw from the circulation or the body fluid.

In certain embodiments, the anti-HLA-E antibodies are immunoreactive to less than five HLA-A alleles and to more than five HLA-B and HLA-Cw alleles.

In some embodiments, the immunoreactivity of the anti-HLA-E antibodies as well as their immunoreactivity to HLA Ia can be blocked by peptide sequences of HLA-E shared with other HLA Ia alleles. The polypeptides comprising the amino acid sequences QFAYDGKDY (SEQ ID NO: 5) and DTAAQI (SEQ ID NO: 8) effectively block anti-HLA-E monoclonal antibodies. See Ravindranath et al., 2010, Mol. Immunology 47: 1121-1131. In some embodiments, the immunoreactivity of the anti-HLA-E antibodies can be blocked by polypeptides comprising the amino acid sequences QFAYDGKDY (SEQ ID NO: 5), LNEDLRSWTA (SEQ ID NO: 7) and/or DTAAQI (SEQ ID NO: 8). Ravindranath. et al., 2011, Mol. Immunol. 48: 423-430. In some embodiments, the immunoreactivity of the anti-HLA-E antibodies can be blocked by polypeptides comprising the amino acid sequence EYWDRETR (SEQ ID NO: 2). In some embodiments, the immunoreactivity of the anti-HLA-E antibodies can be blocked by poly peptides comprising the amino acid sequence EPPKTHVT (SEQ ID NO: 12). In some embodiments, the immunoreactivity of the anti-HLA-E antibodies can be blocked by polypeptides comprising the amino acid sequence RAYLED (SEQ ID NO: 10). See Ravindranath et al., 2010, J. Immunology 185(3): 1935-48. In some embodiments. the immunoreactivity of the anti-HLA-E antibodies can be blocked by polypeptides comprising the amino acid sequence RSARDTA (SEQ ID NO: 13). In some embodiments, the immunoreactivity of the anti-HLA-E antibodies can be blocked by polypeptides comprising the amino acid sequence SEQKSNDASE (SEQ ID NO: 14).

In some embodiments, the composition is capable of suppressing naïve and/or activated T-cells in a recipient of the pharmaceutical composition, in a manner similar or identical to that of IVIg. In certain embodiments, the composition is capable of suppressing the proliferation and/or blastogenesis of naïve and/or activated CD 3+/CD4+ T-cells in a recipient of the pharmaceutical composition, in a manner similar or identical to that of IVIg. In certain embodiments, the composition is capable of modulating the proliferation and/or blastogenesis of naïve and/or activated CD 3+/CD8+ T-cells in a recipient of the pharmaceutical composition, in a manner similar or identical to that of IVIg.

In some embodiments, the composition is capable of inducing cell death of naïve and/or activated T-cells in a recipient of the pharmaceutical composition, in a manner similar or identical to that of IVIg. In certain embodiments, the composition is capable of inducing cell death of naïve and/or activated CD 3+/CD4+ T-cells in a recipient of the pharmaceutical composition, in a manner similar or identical to that of IVIg. In certain embodiments, the composition is capable of inducing cell death of activated CD 3+/CD8+ T-cells in a recipient of the pharmaceutical composition, in a manner similar or identical to that of IVIg.

In some embodiments, the pharmaceutical composition is capable of suppressing formation of T-cell dependent HLA antibodies in a recipient. In certain embodiments, the T-cell dependent HLA antibodies are anti-HLA la antibodies. In certain embodiments, the recipient is a transplant recipient.

In some embodiments of the pharmaceutical composition provided herein, the anti-HLA-E antibodies are immunoreactive to HLA la heavy chains and HLA-E heavy chains similar to a commercial preparation of IVIg. In certain embodiments. the anti-HLA-E antibodies are immunoreactive to at least 70% of the same HLA la antigens as IVIg.

In some embodiments, the pharmaceutical composition is therapeutically effective for the treatment of one or more inflammatory diseases or symptoms thereof treatable by commercial preparations of IVIg. In specific embodiments, the pharmaceutical composition is therapeutically effective for the treatment of a graft rejection.

In certain embodiments, the anti-HLA-E antibodies have immunomodulatory activity comparable to commercial preparations of IVIg. In certain embodiments, the anti-HLA-E antibodies modulate T-cell growth, expansion and/or proliferation comparable to a commercial preparation of IVIg.

In another aspect provided herein is a method of preventing, managing, treating and/or ameliorating a graft rejection, the method comprising administering to a mammal a therapeutically effective amount of any one of the pharmaceutical compositions provided herein.

In some embodiments, the method is for the prevention, management, treatment and/or amelioration of a tissue graft rejection. In some embodiments, the method is for the prevention, management, treatment and/or amelioration of an organ graft rejection. In particular embodiments, the organ graft is a heart, kidney or liver graft. In other embodiments, the method is for the prevention, management, treatment and/or amelioration of a cell graft rejection. In particular embodiments, the cell graft is a bone marrow transplantation or a blood transfusion.

In yet another aspect provided herein is a method of managing, treating and/or ameliorating an inflammatory disease or condition selected from the group consisting of: Kawasaki disease, immune-mediated thrombocytopenia, primary immunodeficiencies, hematopoietic stem cell transplantation, chronic B-cell lymphocytic leukemia, pediatric HIV type 1 infection, hematological disease, nephropathy, neuropathy, abacterial infection, a viral infection, an autoimmune disease that is not vasculitis, cardiomyopathy, an eye or ear inflammatory disease. a lung inflammatory disease, recurring pregnancy loss, Behçet syndrome, chronic fatigue syndrome, congenital heart block, diabetes mellitus, acute idiopathic dysautonomia, opsoclonus-myoclonus, Rasmussen syndrome, Reiter syndrome, or Vogt-Koyanagi-Harada syndrome, the method comprising administering to a mammal a therapeutically effective amount of any of the pharmaceutical compositions provided herein.

In some embodiments, at least 80% of the antibodies of the composition are anti-HLA-E antibodies according to the description provided herein. In some embodiments, at least 85% of the antibodies of the composition are anti-HLA-E antibodies. In some embodiments, at least 90% of the antibodies of the composition are anti-HLA-E antibodies. In some embodiments, at least 95% of the antibodies of the composition are anti-HLA-E antibodies. In some embodiments, at least 99% of the antibodies of the composition are anti-HLA-E antibodies.

### BRIEF DESCRIPTION OF THE FIGURES AND TABLES

FIGS. 1A and 1B show that IgG immunoreactive to HLA-E is present in two different commercial sources of IVIg. The levels of IgG immunoreactive to HLA-E are expressed as mean fluorescent intensity (MEI). The level of IgG immunoreactive to HLA-E is high as evidenced at different dilutions. The MFI values increase from dilution 1/2 to 1/32 dilution for one IVIg source (IVIGlob® EX, FIG. 1A) and from 1/2 to 1/8 for IVIg from a different commercial source (GamaSTAN™ S/D, TALECRIS, FIG. 1B). Such increases signify the aggregation of anti-HLA-E reactive IgG at high concentration and also indicates the high titer of anti-HLA-E IgG antibodies in the IVIg preparations.

FIGS. 2A and 2B show that the immunoreactivity to HLA Ia seen in two different commercial sources of IVIg is due to anti-HLA-E antibodies that are immunoreactive to HLA Ia.

FIGS. 3A-3C show that IVIg immunoreactivity to HLA-E and HLA Ia is lost after adsorbing IVIg to Affi-Gel conjugated with HLA-E.

FIG. 4 shows that anti-HLA-E monoclonal antibodies (MAb-1 and MAb-2), which are not immunoreactive to HLA-F and HLA-G, are immunoreactive to HLA-class Ia alleles. It is evident that immunoreactivity to HLA-E accompanies immunoreactivity to HLA Ia as evidenced by the affinity of two different sources of anti-HLA-E monoclonal antibodies.

FIGS. 5A and 5B show that the lectin Phytohemagglutinin (PHA-L) is capable of stimulating human T-Iymphocytes. FIGS. 5A and 5B illustrate the events occurring 70 hrs after PHA-L stimulation of T-Iymphocytes (CD3+/CD4+).

FIGS. 6A and 6B shows that IVIg induces cell death, arrests proliferation and blastogenesis of PHA-L stimulated T-Iymphocytes (CD3+/CD4+).

FIGS. 7A and 7B shows that anti-HLA-E MAb-1 induces cell death, arrests proliferation and blastogenesis of PHA-L stimulated T-lymphocytes (CD3+/CD4+) similar to IVIg.

FIGS. 8A and 8B shows that anti-HLA-E MAb-2 induces cell death, arrests proliferation and blastogenesis of PHA-L stimulated T-lymphocytes (CD3+/CD4+).

FIGS. 9A-9G show that IV inhibits PHA-induced T-cell proliferation identical to anti-HLA-E MAb (MAb-1) as determined by carboxyfluorescein diacetate succinimidyl ester (CFSE) staining technology.

FIG. 9A depicts a profile of the CFSE fluorescence intensity of proliferating T-cells after 70 hours of exposure to PHA-L. The profile closely follows the predicted sequential halving due to cell division (M1, M2, M3 and M4).

FIG. 9B shows the inhibition of PHA-L induced proliferation of CD3+ CFSE+T-lymphocytes by IVIg at 72 hrs.

FIG. 9C depicts the inhibition of PHA-L induced proliferation of CD3+ CFSE+T-lymphoblasts by IVIg at 72 hrs.

FIG. 9D depicts the percentage of inhibition of T-cell proliferation by IVIg at different dilutions, 72 hrs after PHA-L stimulation.

FIG. 9E depicts the inhibition of PHA-L induced proliferation of CD3+ CFSE+T-lymphocytes by anti-HLA-E MAb-1 at 72 hrs.

FIG. 9F depicts the inhibition of PHA-L induced proliferation of CD3+ CFSE+T-lymphoblasts by anti-HLA-E MAb-1 at 72 hrs.

FIG. 9G depicts the percentage of inhibition of T-cell proliferation by anti-HLA-E MAb-1 at different dilutions, 72 hrs after PHA-L stimulation.

FIG. 10 shows that IVIg dosimetrically inhibits PHA-L stimulated CD4+ T-lymphocytes and T-lymphoblasts.

FIG. 11 shows that anti-HLA-E MAb-1 dosimetrically inhibits PHA-L stimulated CD4+ T-lymphocytes and T-lymphoblasts.

FIG. 12 shows that IVIg inhibits PHA-L stimulated blastogenesis but promotes proliferation of CD8+ T-lymphocytes.

FIG. 13 shows that anti-HLA-E monoclonal antibody MAb-1 inhibits PHA-L stimulated blastogenesis but promotes proliferation of CD8+ T-lymphocytes.

FIG. 14 depicts the similarities between IVIg and anti-HLA-E MAb-1 in the dose dependent inhibition of PHA-L stimulated proliferation and blastogenesis of CD4+ T-cells on one hand and the failure to inhibit proliferation of PHA-L stimulated CD8+ T-cells on the other hand. The differences in the dilutions show the differences in the potency between IVIg and anti-HLA-E Ab. Anti-HLA-E Ab, though functionally similar to IVIg, seems to be more potent than IVIg.

FIG. 15 depicts the presence of soluble HLA-E in the sera of liver transplant recipients (TFL-Michigan Sera: Patient ID: I, Mi-9707, 2, Mi-11151, 3, Mi-11553, 4, Mi-10788, 5. Mi-11909, 6, Mi-12172. 7, Mi-13041, 8, Mi-13100) as shown through Western blots immunostained with MAbs MEM-E/02 (A) or MEM-E/06 (B).

FIG. 16 depicts Western blots of electropherograms showing the presence of soluble HLA-E in the sera of kidney transplant recipients as shown through Western blots. Western blots of electropherograms were obtained without (A) or with (B) reducing agents and immunostained with MAb MEM-E/02.

FIG. 17 depicts the various immunodulatory effects thought to be provided by IVIg. These immunomodulatory activities of IVIg are thought to include, but are not limited to, modulation of T-cell, B-cell and dendritic cell growth, expansion or proliferation, downregulation of expression of MHC class II molecules, inhibition of expression of CD80/CD86 molecules, suppression of dendritic cell-mediated activation and proliferation of alloreactive T-cells, induction of apoptosis of T-cells, suppression of the expansion of autoreactive B-cells, inhibition of complement activation, and enhancement of clearance of endogenous pathogenic autoantibodies.

Table 1 depicts the characteristics of five different commercial preparations of IVIg. The number of donors used for each commercial preparation differs although all the commercial preparations follow the guidelines recommended by WHO. According to the WHO requirements for intravenous immunoglobulin preparations, IVIg should be extracted from a pool of at least 1000 individual donors. Non-paid donors are preferred by many manufacturers, since paid donors increase the risks of viral and other contaminants. The IgG preparations should contain at least 90% intact IgG and as small an amount of IgA concentration as possible, as well as being free from fragments and aggregates. IVIg should be modified biochemically as little as possible and should possess opsonising and complement fixing characteristics as well as other natural biological characteristics. All IgG subclasses should be present, whenever possible, in similar distributions as in normal human plasma. The immunoglobulins should meet WHO standards and be free from prekallikrein activator, kinins, plasmins, accumulating preservatives (stabilizers) and other damaging contaminants as far as possible.

Table 2 summarizes the FDA approved uses for different commercial preparations of IVIg. The FDA has approved selected commercial IVIg preparations for certain diseases. The basis for an FDA licensure of solvent detergent process include viral inactivation of antihemophilic factor (AHF), demonstrated inactivation of marker viruses, effects against lipid-enveloped viruses , and paucity of adverse effect of solvent detergent and AHF proteins. Solvent detergent process is virucidal for VSV (vesicular stomatitis virus), Sindbis virus, HIV (human immunodeficiency virus), HBV (hepatitis B virus) and HCV (hepatitis C virus NANBHV). The following proteins are not affected by Antihemophilic Factor (AHF): Factor VIII, Factor IX, Fibrin, Fibrinogen, IgG and IgM. Based on these and other evaluations, the FDA recommends selected commercial preparations for certain diseases.

Table 3 depicts peptide sequences of HLA-E shared and not shared (*) by other Class Ia and Ib alleles. The heavy chains of classical FILA class Ia (HLA-A, -B and -C) and non-classical HLA-E share several peptide sequence similarities. However, two peptide sequences (*) are unique to HLA-E and are not found in any of the HLA Ia alleles or HLA-F and HLA-G alleles. Theoretically, an anti-HLA-E specific monoclonal antibody can be expected to bind only to these two peptide sequences but not to other shared sequences. Since HLA-E share several peptide sequence similarities with the heavy chains of classical HLA class Ia (HLA-B and -C) molecules, we hypothesized that the antibodies to HLA-E that recognize shared sequences, may bind to HLA la alleles. This hypothesis is tested by examining the affinity of HLA-E monoclonal antibodies (HLA-E-MAbs) to HLA Ia molecules and by inhibiting the antibody binding to both HLA-E and HLA-Ia with the shared peptide sequence(s) See Ravindranath et al., 2010, Mol. Immunol. 47: 1121-1131; Ravindranath, et al., 2011, Mol. Immunol. 48: 423-430.

Table 4 demonstrates that soluble HLA-E in the sera of liver allograft recipients (Mi127, Mi114. Mi92 & Mi59; sera diluted 1/100) was able to inhibit HLA Ia reactivity of the murine monoclonal antibody (MAb) MEM-E/02. Inhibition is expressed as percentage inhibition of Mean Fluorescent Intensity (MFI) of the MEM-E/02.

Table 5 demonstrates that different dilutions of soluble HLA-E in the IgG-free serum of a liver allograft recipient (Mi 92) inhibited HLA-Ia reactivity of the murine monoclonal antibody (MAb) MEM-E/02. The inhibition is compared with that of HLA-E. The values are expressed as Mean Fluorescent Intensity (MFI) of the MAb.

### DETAILED DESCRIPTION OF THE EMBODIMENTS

Definitions

As used herein, “administer” or “administration” refers to the act of injecting or otherwise physically delivering a substance as it exists outside the body (e.g., a pharmaceutical composition described herein) into a patient, such as by, but not limited to, pulmonary (e.g., inhalation), mucosal (e.g., intranasal), intradermal, intravenous, intramuscular delivery and/or any other method of physical delivery described herein or known in the art. When a disease. or symptoms thereof, is being treated, administration of the substance typically occurs after the onset of the disease or symptoms thereof. When a disease, or symptoms thereof, is being prevented. administration of the substance typically occurs before the onset of the disease or symptoms thereof.

The term “antibodies that are immunoreactive” to a particular human leukocyte antigen (HLA) refer to antibodies that specifically bind to a particular HLA. For example, “antibodies immunoreactive to HLA-E” refers to antibodies, including both modified antibodies and unmodified antibodies that specifically bind to an HLA-E polypeptide heavy chain polypeptide). An antibody or a fragment thereof is immunoreactive to a particular HLA or HLAs when it binds to the particular HLA or HLAs determined using experimental immunoassays known to those skilled in the art. Immunoassays combine the principles of immunology and biochemistry enabling tests, which include but are not limited to RIAs (radioimmunoassays), enzyme immunoassays like ELISAs (enzyme-linked immunosorbent assays), LIAs (Luminescent immunoassays) and FIAs (fluorescent immunoassays). Antibodies used in the aforementioned assays, for instance primary or secondary antibodies, can be labeled with radioisotopes (e.g., ¹²⁵I), fluorescent dyes (e.g., PC or FITC) or enzymes (e.g., peroxidase or alkaline phosphatase), which catalyze fluorogenic or luminogenic reactions. See e.g., Eleftherios et al., 1996, Immunoassay, Academic Press; Law et al., 2005, Immunoassay: A Practical Guide, Taylor & Francis; Wild et al., 2005, The Immunoassay Handbook, Third Edition, Elsevier; Paul et al., 1989, Fundamental Immunology, Second Edition, Raven Press, for a discussion regarding antibody specificity.

In general, an antibody immunoreactive to HLA-E can bind to HLA-E alleles. Antibodies immunoreactive to a particular HLA allele (e.g., an HLA-E allele) can specifically bind to a polypeptide comprising the amino acid sequence of that particular HLA allele and to other HLA alleles if the other HLA alleles share the amino acid sequence and physical conformation of the same polypeptide found in said particular HLA allele (e.g., an HLA-E allele). See Ravindranath et al., 2010, Mol, Immunol. 47: 1121-1131; Ravindranath et al., 2011, Mol. Immunol. 48: 423-430.

Antibodies provided herein include any form of antibody known to those skilled in the art. Antibodies provided herein include both modified antibodies (i.e., antibodies that comprise a modified IgG (e.g., IgG1) constant domain, or FcRn-binding fragment thereof, (e.g., the Fc-domain or hinge-Fc domain)) and unmodified antibodies (i.e., antibodies that do not comprise a modified IgG (e.g., IgG1) constant domain). Antibodies provided herein include, but are not limited to, synthetic antibodies, monoclonal antibodies, polyclonal antibodies, recombinantly produced antibodies, human antibodies, humanized antibodies. chimeric antibodies, intrabodies, single-chain Fvs (scFv) (e.g., including monospecific, bispecific, etc.). Fab fragments, F(ab′) fragments, disulfide-linked Fvs (sdFv), anti-idiotypic (anti-Id) antibodies, and epitope-binding fragments of any of the above. In particular. antibodies include immunoglobulin molecules and immunologically active portions of immunoglobulin molecules.

The term “antigen,” with respect to HLAs, refers to an HLA heavy chain or portion of an HLA heavy chain that is bound to another HLA heavy chain to form a homodimer, or an HLA heavy chain or portion of an HLA heavy chain associated with a β2-microglobulin to form a heterodimer or an HLA heavy chain or portion of an HLA heavy chain that is free (i.e., not bound to another HLA or β2-microglobulin). HLA antigens include those expressed or located on a cell surface or those occurring in soluble form in circulation or body fluids.

Antibodies provided herein can be of any subclass of IgG (e.g., IgG1, IgG2 (IgG2a and IgG2b), IgG3, IgG4).

The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “effective amount” as used herein refers to the dose or amount required for treatment (e.g., an antibody provided herein) which is sufficient to reduce and/or ameliorate the severity and/or duration of any one of the disease or conditions described herein. In some embodiments, the effective amount of an antibody of the pharmaceutical composition provided herein is between about 0.025 mg/kg and about 60 mg/kg body weight of a human subject. In some embodiments. the effective amount of an antibody of the pharmaceutical composition provided herein is about 0.025 mg/kg or less, about 0.05 mg/kg or less, about 0.10 mg/kg or less, about 0.20 mg/kg or less, about 0.40 mg/kg or less, about 0.80 mg/kg or less, about 1.0 mg/kg or less, about 1.5 mg/kg or less, about 3 mg/kg or less, about 5 mg/kg or less, about 10 mg/kg or less, about 15 mg/kg or less, about 20 mg/kg or less, about 25 mg/kg or less, about 30 mg/kg or less, about 35 mg/kg or less, about 40 mg/kg or less. about 45 mg/kg or less, about 50 mg/kg or about 60 mg/kg or less.

The term “epitopes” as used herein refers to continuous or discontinuous peptide sequence or sequences or fragments of a polypeptide (e.g., an HLA-E, HLA-F or HLA-G a chain polypeptide) recognized by the Fab portion of the antibody, and having immunogenic activity in an animal, preferably a mammal, and most preferably in a human. An epitope having immunogenic activity is a fragment of a polypeptide that elicits an antibody response in an animal or in a human. See Table 3 for epitope sequences of HLA-E.

The term “excipients” as used herein refers to inert substances which are commonly used as a diluent, vehicle, preservatives, binders, or stabilizing agent for drugs and includes, but not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). Also see Remington et al., 1990, Remington's Pharmaceutical Sciences, Mack Publishing Co, which is hereby incorporated in its entirety.

In the context of a peptide or polypeptide, the term “fragment” as used herein refers to a peptide or polypeptide comprising an amino acid sequence of at least 5 contiguous amino acid residues, at least 10 contiguous amino acid residues, at least 15 contiguous amino acid residues of the amino acid sequence of a particular polypeptide to which an antibody immunospecifically binds.

The terms “IgG Fc region,” “Fc region,” “Fc domain,” “Fc fragment” and other analogous terms as used herein refer the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but may or may not contain carbohydrate moiety and the binding sites for complement and Fc receptors, including the FcRn receptor (see below).

The term “immunomodulatory agent” and variations thereof including, but not limited to, immunomodulatory agents, as used herein refer to an agent that modulates one or more of the components (e.g., immune cells, or subcellular factors, genes regulating immune components, cytokines, chemokines or such molecules) of a host's immune system. In certain embodiments, an immunomodulatory agent is an immunosuppressive agent. In certain other embodiments, an immunomodulatory agent is an immunostimulatory agent. Immunomodulatory agents may include, but are not limited to, small molecules, peptides, polypeptides, proteins, fusion proteins, antibodies, inorganic molecules, mimetic agents, and organic molecules.

An “isolated”- or “purified” antibody is substantially free of cellular material or other contaminating proteins or other antibodies. The language “substantially free of cellular material” includes preparations of an antibody in which the antibody is separated from cellular components of the cells from which it is isolated or recombinantly produced. When the antibody is recombinantly produced, it can also be substantially free of culture medium. When the antibody is produced by chemical synthesis, it can also be substantially free of chemical precursors or other chemicals, i.e., it is separated from chemical precursors or other chemicals which are involved in the synthesis of the protein. In a specific embodiment, antibodies provided herein are isolated or purified.

As used herein, the terms “manage,” “managing,” and “management” refer to the beneficial effects that a subject derives from a therapy (e.g., a prophylactic or therapeutic agent), which does not result in a cure of the disease or condition described herein.

As used herein, the term “modified antibody” encompasses any antibody described herein that comprises one or more “modifications” to the amino acid residues at given positions of the antibody constant domain (e.g., an IgG or an IgG1 constant domain), or FcRn-binding fragment thereof wherein the antibody has an increased in vivo half-life as compared to known antibodies and/or as compared to the same antibody that does not comprise one or more modifications in the IgG constant domain, or FcRn-binding fragment thereof. As used herein, a “modified antibody” may or may not be a high potency, high affinity and/or high avidity modified antibody. In certain embodiments, the modified antibody is a high potency antibody. In certain embodiments, the modified antibody is a high potency, high affinity modified antibody.

The term “pharmaceutically acceptable” as used herein means being approved by a regulatory agency of the Federal or a state government, or listed in the U.S. Pharmacopia, European Pharmacopia or other generally recognized pharmacopia for use in animals, and more particularly in humans.

As used herein, the terms “prevent,” “preventing,” and “prevention” refer to the total or partial inhibition of any of the diseases or conditions described herein.

The terms “stability” and “stable” as used herein in the context of a liquid formulation comprising an antibody provided herein refer to the resistance of the antibody in the formulation to thermal and chemical unfolding, aggregation, degradation or fragmentation under given manufacture, preparation, transportation and storage conditions. The “stable” formulations of the antibodies and pharmaceutical compositions provided herein retain biological activity equal to or more than 80%, 85%, 90%, 95%, 98%, 99%, or 99.5% under given manufacture, preparation, transportation and storage conditions. The stability of the antibody can be assessed by degrees of aggregation, degradation or fragmentation by techniques known to those skilled in the art, including but not limited to reduced Capillary Gel Electrophoresis (rCGE), Sodium Dodecyl Sulfate Polyacrylamide Gel Electrophoresis (SDS-PAGE) and HPSEC. The overall stability of a formulation comprising an antibody that immunospecifically binds to an HLA-E antigen can be assessed by various immunological assays including, for example, ELISA and radioimmunoassay using the entire or part of the polypeptide of HLA-E.

As used herein, the terms “subject” and “patient” are used interchangeably. In some embodiments, the subject is a human and in others it is an animal.

The term “substantially free of surfactant” as used herein refers to a formulation of a pharmaceutical composition, said formulation containing less than 0.0005%, less than 0.0003%, or less than 0.0001% of surfactants and/or less than 0.0005%, less than 0.0003%, or less than 0.0001% of surfactants.

The term “substantially free of salt” as used herein refers to a formulation of a pharmaceutical composition, said formulation containing less than 0.0005%, less than 0.0003%, or less than 0.0001% of inorganic salts.

The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials.

As used herein, the term “therapeutic agent” refers to any agent that can be used in the treatment, management or amelioration of one of the diseases or conditions described herein.

As used herein, the term “therapy” refers to any protocol, method and/or agent that can be used in the prevention, management, treatment and/or amelioration of one of the diseases or conditions described herein.

In certain embodiments provided herein, the term “therapeutically effective” with respect to the pharmaceutical composition, refers to the ability of the composition to reduce the severity, the duration and/or the symptoms of a particular disease or condition.

As used herein. the terms “treat,” “treatment” and “treating” refer to the reduction or amelioration of the progression, severity, and/or duration of one of the conditions described herein.

Antibodies and Pharmaceutical Compositions

Provided herein are chimeric, humanized or human anti-HLA-E IgG antibodies immunoreactive to the heav) chain polypeptide of HLA-E and not immunoreactive to the heavy chain polypeptide of HLA-F or HLA-G. Also provided herein are pharmaceutical compositions comprising said antibodies in a pharmaceutically acceptable carrier.

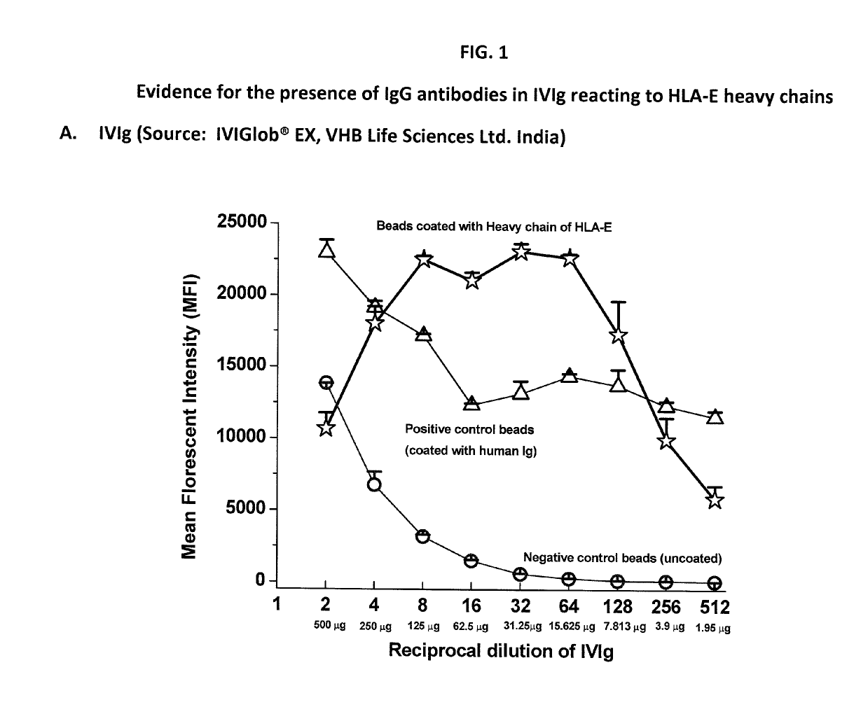

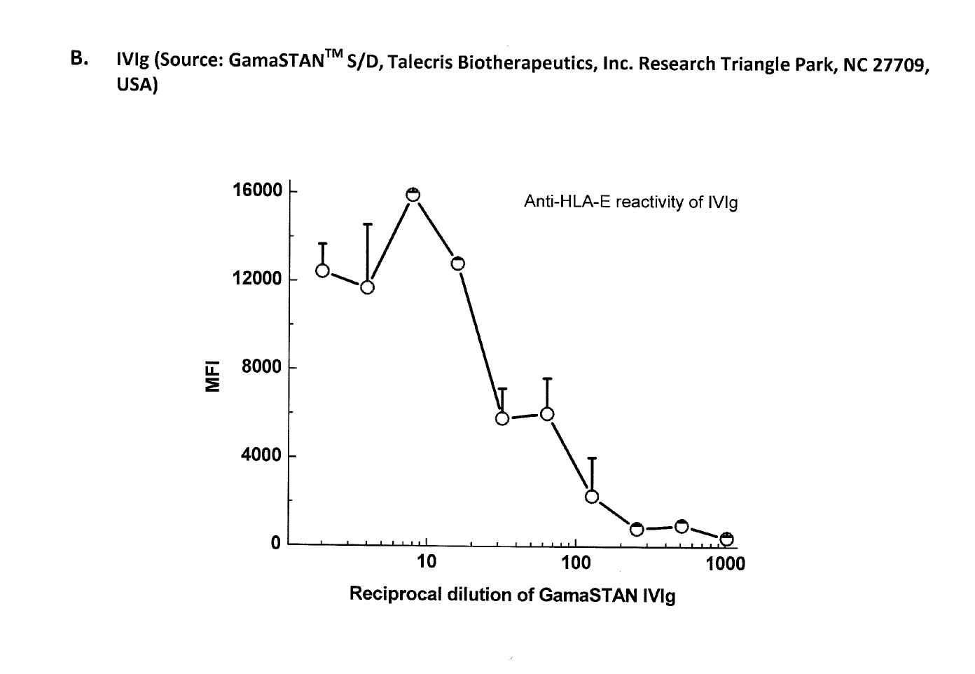

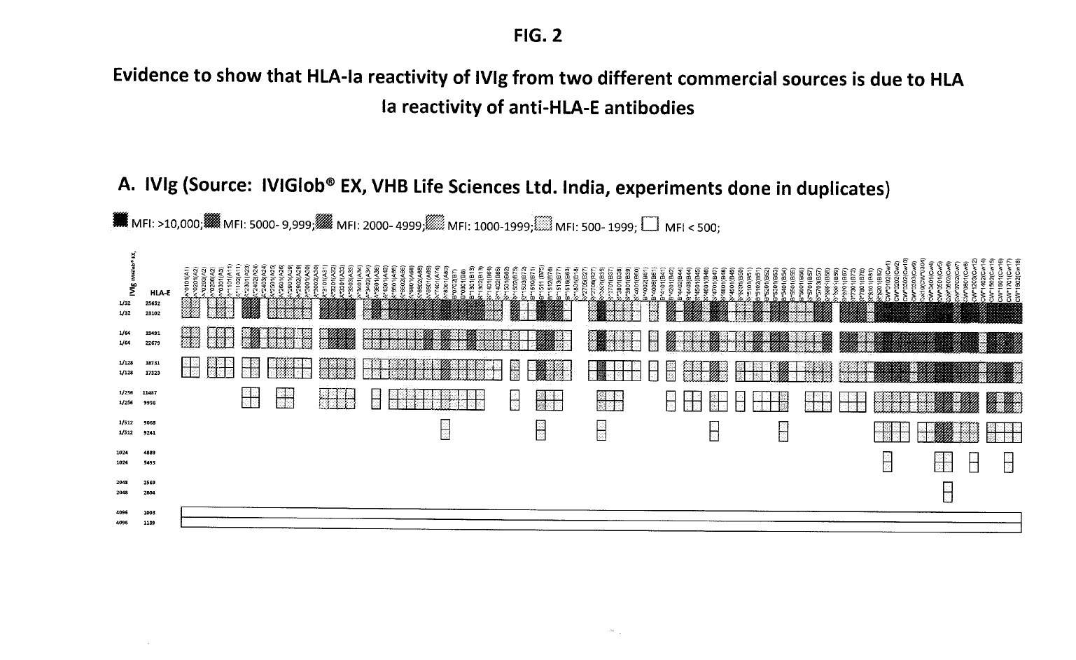

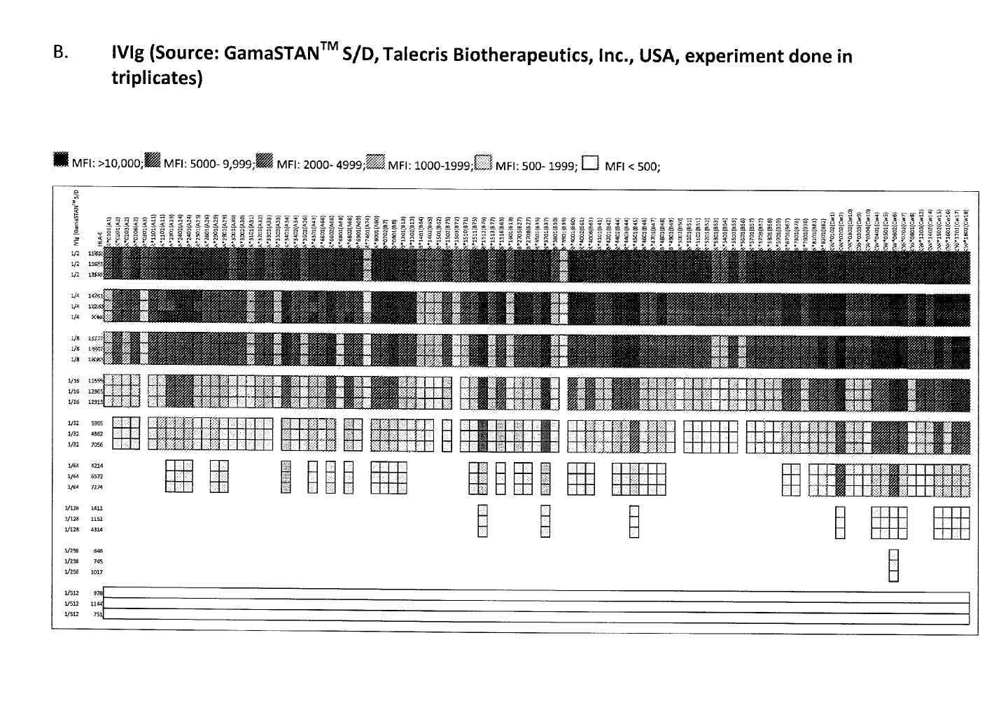

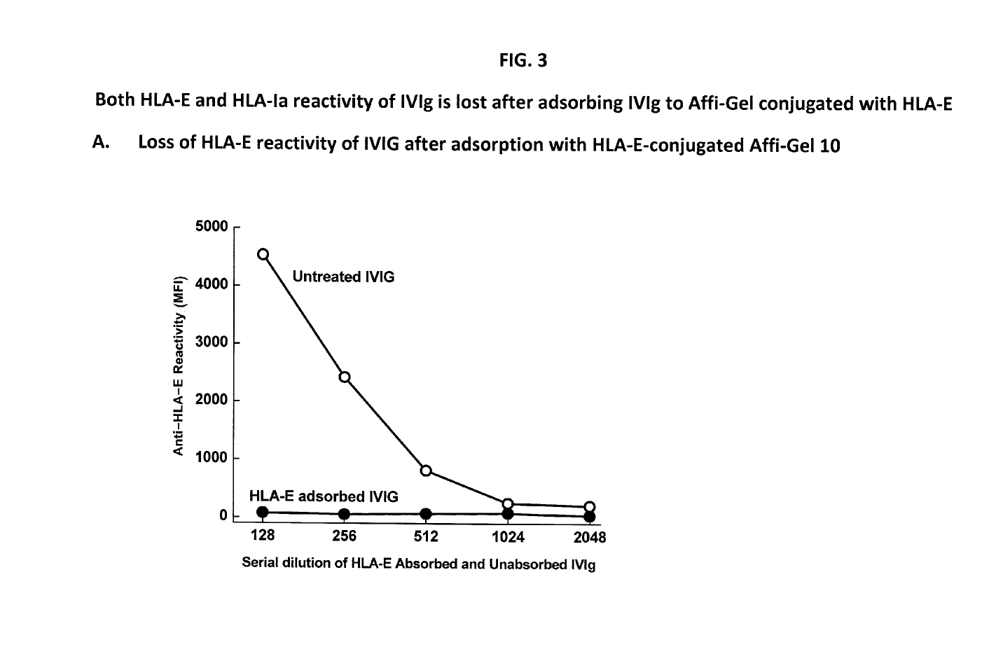

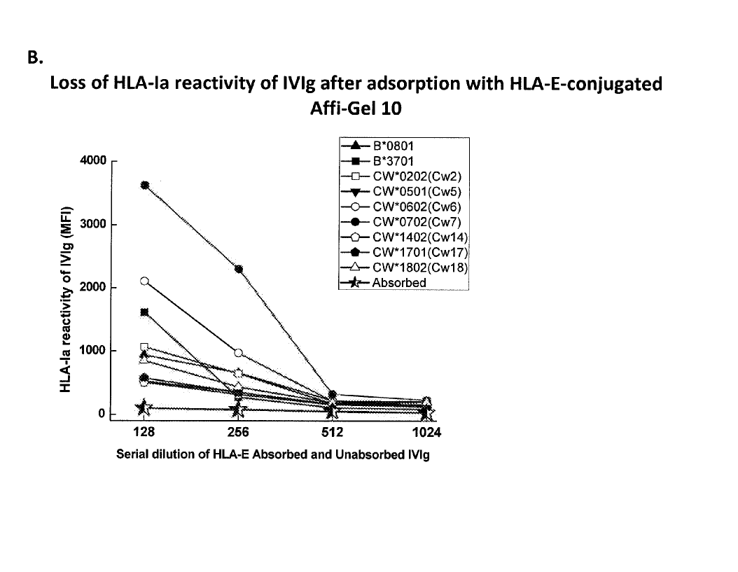

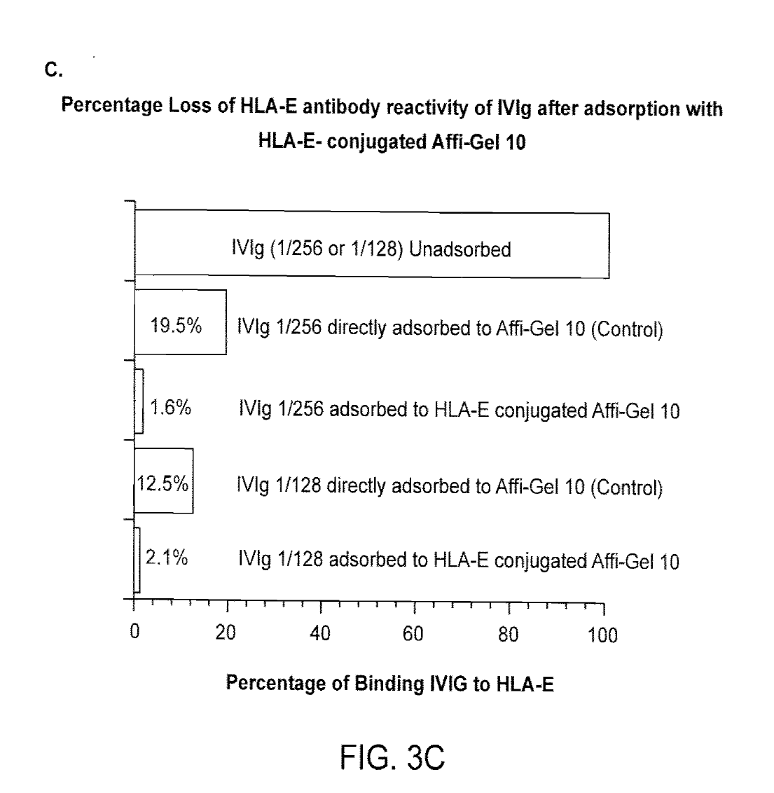

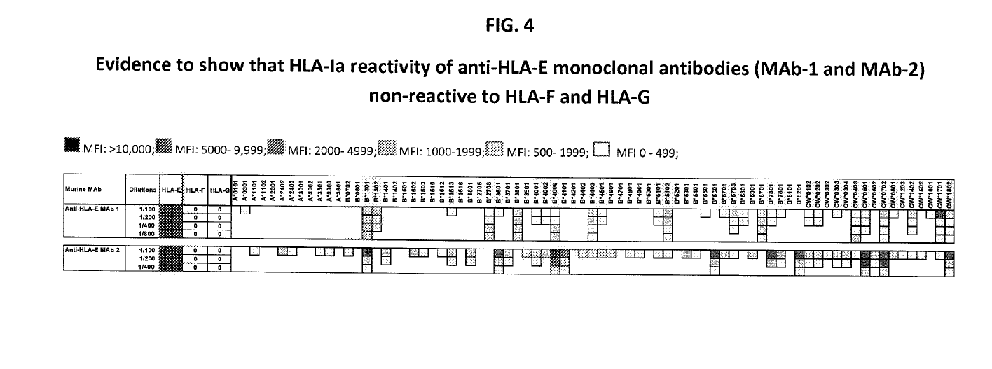

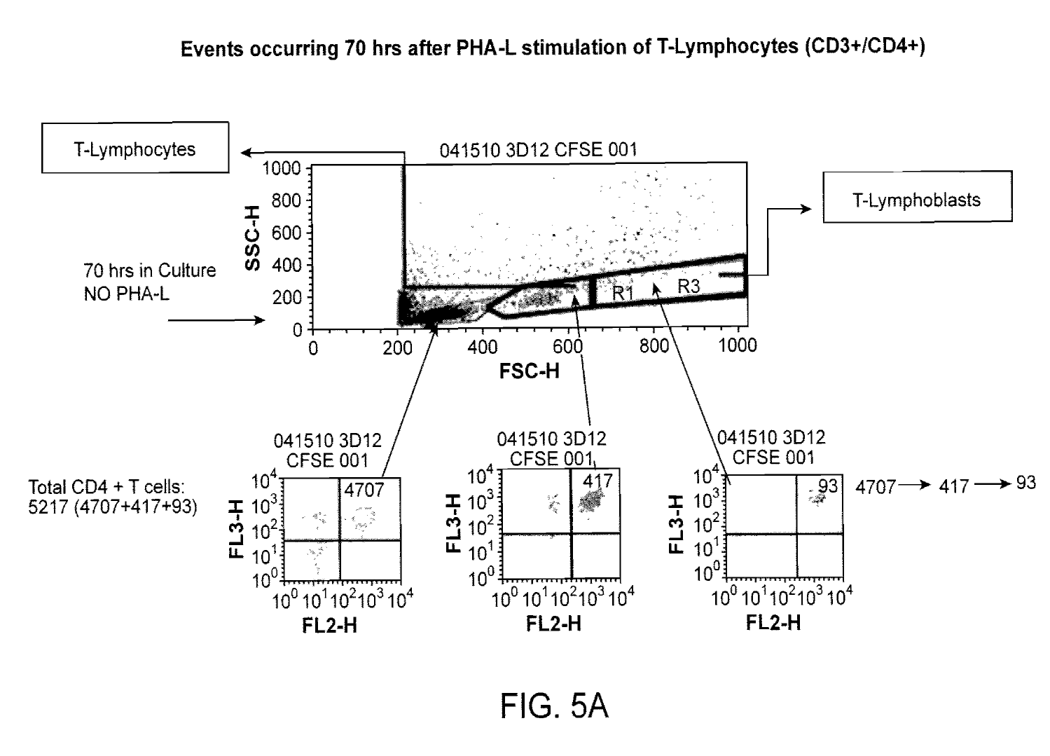

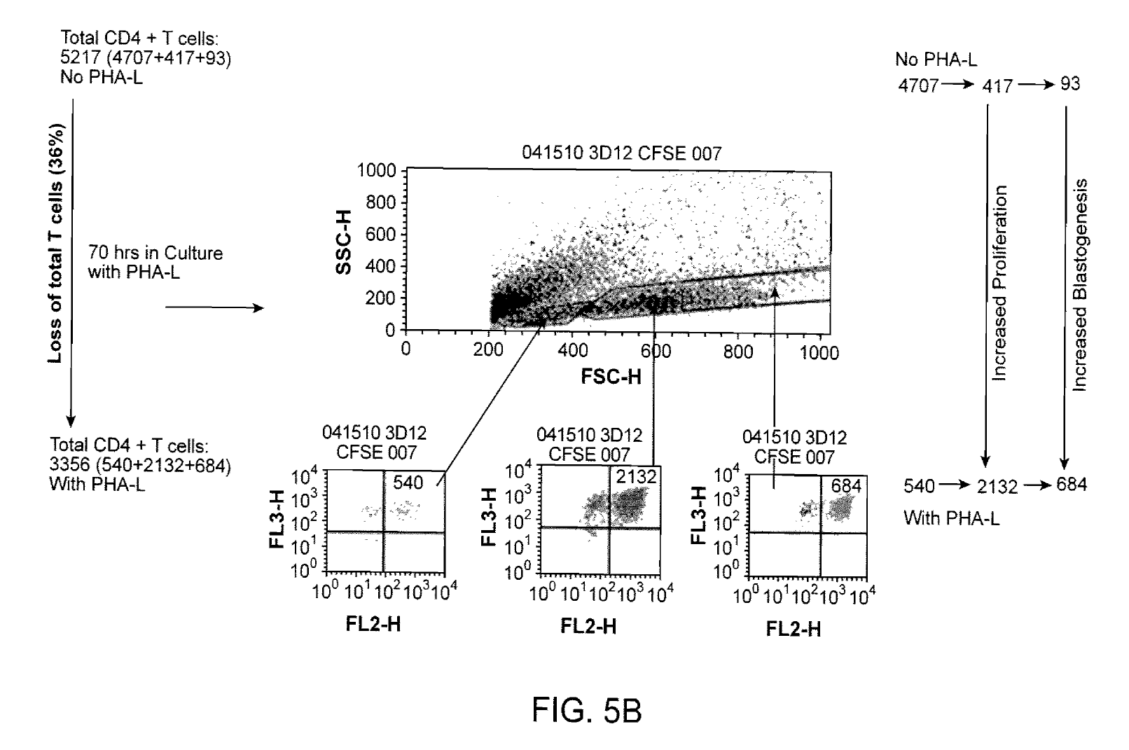

Without being bound to any particular theory of operation, it is believed that a pharmaceutical composition comprising anti-HLA-E antibodies can mimic the therapeutic effects of whole IVIg. For instance, our observations show that IVIg comprises high levels of anti-HLA-E antibodies (FIG. 1) and IVIg is as immunosuppressive as anti-HLA-E antibodies (FIGS. 5 to 14). For example, anti-HLA-E antibodies have been demonstrated to inhibit the proliferation and blastogenesis of CD4+ and CD8+ T-cells in a manner similar to whole IVIg (FIGS. 5 to 14). Indeed, IVIg depleted of anti-HLA-E antibodies no longer exhibits HLA Ia reactivity (FIG. 3A, B and C). Most importantly, IVIg mimics the immunoreactivity of anti-HLA-E antibodies to HLA la alleles (FIG. 2 and FIG. 4). Thus, it is believed that compositions comprising anti-HLA-E antibodies can advantageously be used as cost effective IVIg substitutes for the prevention, treatment, therapy and/or amelioration of particular diseases while minimizing IVIg related side effects.

The pharmaceutical composition can be made by any technique apparent to one of skill in the art, including the techniques described herein. Each element of the pharmaceutical composition is discussed in further detail below.

Anti-HLA-E Antibodies

Provided herein are chimeric, humanized or human anti-HLA-E IgG antibodies immunoreactive to the heavy chain polypeptide of HLA-E and not immunoreactive to the heavy chain polypeptide of HLA-F or HLA-G.

Major Histocompatibility Class 1 (MHC-I) molecules include highly polymorphic classical HLA class Ia (HLA-A alleles [n=767], HLA-B alleles [n=1178], HLA-C alleles [n=439]) and less polymorphic non-classical HLA Ib (HLA-E alleles [n=9], HLA-F alleles [n=21], HLA-G alleles [n=43]) (Geraghty et al., 1987, Proc. Natl. Acad. Sci. U.S.A. 84: 9145-9149; Geraghty et al., 1990, J. Exp. Med. 171: 1-18; Koller et al., 1988, J. Immunol. 141: 897-904; Shawar et al., 1994, Ann. Rev. Immunol. 12: 839-880).

HLA Ia molecules are co-dominantly expressed on the cell membrane as pair of alleles for each of the three HLA-Ia molecules. HLA Ia molecules can bind and present peptide antigens produced intracellularly, including viral and tumor specific proteins, to CD8+ effector T-cells (e.g., cytotoxic T-cells (CTLs)). In response to foreign antigens presented by HLA Ia bearing cells. CD8+ effector T-cells can destroy the cells presenting the foreign antigen.

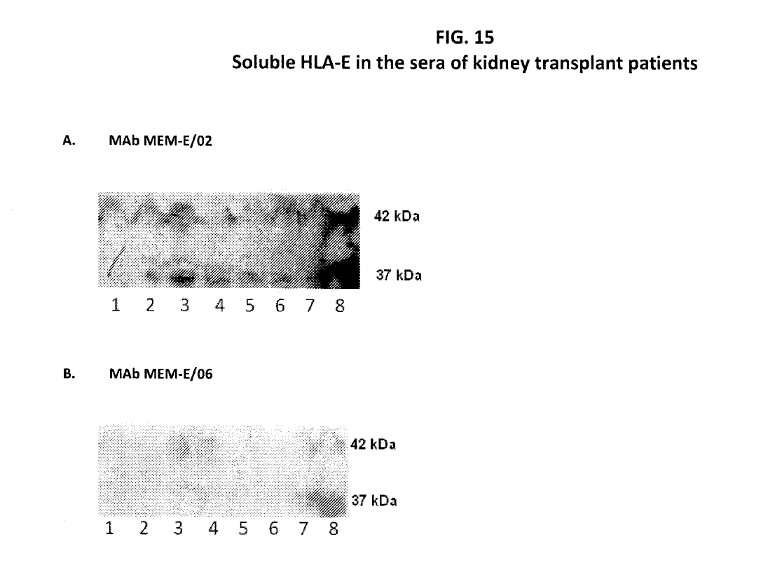

Each HLA-I molecule, when expressed on a cell surface. may consist of a heavy chain (HC) (of about 346 amino acids) that is free, an HC linked to an HC of the same allele or an HC non-covalently linked to 132-microglobulin (“β₂m”) (99 amino acids). HC consists of three extracellular domains (α1, α2 & α3), a transmembrane domain and a C-terminal cytoplasmic domain. However, HLA la molecules can also be expressed without β2m on the cell surface on activated T-lymphocytes (see Schnabel et al., 1990, J. Exp. Med. 171: 1431-1432, CD14+ blood monocytes, activated dendritic cells (see Raine et al., 2006, Rheumatology 45: 1338-1344) of healthy individuals and in cells and tissues of patients with inflammatory diseases (see Raine et al., 2006, Rheumatology 45: 1338-1344; Tsai et al., 2002, Rheumatology 29: 966-972). On the cell surface, HC and β2m can dissociate, leaving membrane bound HC only (Machold, et al., 1996, J. Exp. Med. 184: 2251-2259; Carreno et al., 1994, Eur. J. Immunol. 24: 1285-1292; Parker et al.,1992, J. Immunol. 149: 1896-1904). On the cell surface, the HC of MHC class I can occur in different conformations (Marozzi et al. 1996, Immunogenetics, 43: 289-295). The HC of HLA-I molecules are released from the cell surface into surrounding media and circulation (Demaria et al., 1994, Biol. Chem. 269:6689-6694). In circulation, in blood and in other body fluids, HLA I molecules occur as soluble fraction (heavy chains free or associated with β2-microglobulin) of different molecular weights (47, 42, 35 kDa). See FIGS. 15A and 15B, Table 4 and Table 5. Soluble HLA I can trigger cell death of CD8+ cytotoxic T-Iymphocytes and natural killer cells impair natural killer cell functions. See Demaria et al., 1993, Int J Clin Lab Res. 23:61-9; Puppo et al., 2000. Int Immzinol. 12:195-203; Puppo et al., 2002, ScientificWorldIournal. 2:421-3; Contini et al., 2000, Hum Immunol. 61:1347-51; Contini et al,, 2003, Eur J Immunol. 33:125-34; Spaggiari et al., 2002, Blood 99:1706-14; Spaggiari et al., 2002, Blood 100:4098-107).

Anti-HLA-E antibodies described herein are immunoreactive to HLA-E and not immunoreactive to HLA-F or HLA-G. See, Example 4 and FIG. 4. An antibody is immunoreactive to a particular HLA or HLAs when it binds to the particular HLA or HLAs as determined using experimental immunoassays known to those skilled in the art including, but not limited to, RIAs (radioimmunoassays), enzyme immunoassays like ELISAs (enzyme-linked immunosorbent assays). LIAs (luminescent immunoassays) and FIAs (fluorescent immunoassays), in which the antibodies, either used as primary or secondary antibodies, can be labeled with radioisotopes (e.g., ¹²⁵I), fluorescent dyes (e.g., PC or FITC) or enzymes (e.g., peroxidase or alkaline phosphatase) that catalyze fluorogenic or luminogenic reactions.

Anti-HLA-E IgG antibodies can be produced by any methods known in the art for the synthesis of antibodies, in particular, by chemical synthesis or by recombinant expression techniques. These methods employ. unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al., 1982, Molecular Cloning: A Laboratory Manual, Cold Spring Harbor Laboratory Press; Sambrook et al., 1989, Molecular Cloning: A Laboratory Manual, Second Edition, Cold Spring Harbor Laboratory Press; Ausubel et al., 1987 and annual updates, Current Protocols in Molecular Biology, John Wiley & Sons; Gait ed., 1984, Oligonucleotide Synthesis: A Practical Approach, IRL Press; Eckstein ed., 1991, Oligonucleotides and Analogues: A Practical Approach, IRL Press; Birren et al., 1999, Genome Analysis: A Laboratory Manual, Cold Spring Harbor Laboratory Press.

Chimeric antibodies described herein can be produced by any technique known to those of skill in the art. See, e.g., Morrison, 1985, Science 229: 1202; Oi et al., 1986, BioTechniques 4: 214; Gillies et al., 1989, J. Immunol. Methods 125: 191-202; and U.S. Pat. Nos. 5,807,715; 4,816,567; 4,816,397; and 6,331,415, which are incorporated herein by reference in their entirety.

Human antibodies described herein can be produced by any method known in the art, including but not limited to methods described in PCT Publication Nos. WO 98/24893, WO 96/34096, and WO 96/33735; and U.S. Pat. Nos. 5,413.923; 5,625,126; 5,633.425; 5,569,825; 5,661.016; 5.545,806; 5,814,318; and 5,939,598, which are incorporated by reference herein in their entirety.

Humanized antibodies described herein can be produced using any technique known in the art, including but not limited to, CDR-grafting (European Patent No. EP 239,400; International Publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5): 489-498; Studnicka et aL, 1994. Protein Engineering 7(6): 805-814; and Roguska et al., 1994, PNAS 91; 969-973), chain shuffling (U.S. Pat. No. 5,565,332), and techniques disclosed in, e.g., U.S. Pat. No. 6,407,213; U.S. Pat. No. 5,766,886; WO 9317105; Tan et al., 2002, J. Immunol. 169: 1119 25; Caldas et al., 2000, Protein Eng. 13(5): 353-60; Morea et al., 2000, Methods 20(3): 267 79; Baca et al., 1997, J. Biol. Chem. 272(16): 10678-84: Roguska et al., 1996, Protein Eng. 9(10): 895 904; Couto et al., 1995, Cancer Res. 55 (23 Supp): 5973s- 5977s; Couto et al., 1995, Cancer Res. 55(8): 1717-22; Sandhu, 1994. Gene 150(2): 409-10; and Pedersen et al., 1994, J. Mol. Biol. 235(3): 959-73. See also U.S. Patent Pub. No. US 2005/0042664 A1 (Feb. 24, 2005), which are incorporated by reference herein in its entirety.

In some embodiments, the anti-HLA-E antibodies are purified antibodies. Purified antibodies are substantially free of cellular material or other contaminating proteins from the cell or tissue source from which the protein is derived, or substantially free of chemical precursors or other chemicals when chemically synthesized. Methods of purifying antibodies are well known to those skilled in the art.

The anti-HLA-E antibodies provided herein include, but are not limited to, synthetic antibodies, monoclonal antibodies, polyclonal antibodies recombinantly produced antibodies, multispecific antibodies, single-chain Fvs (scFvs), Fab fragments, F(ab′) fragments, disulfide-linked Fvs (sdFv), anti-idiotypic (anti-Id) antibodies, and epitope-binding fragments of any of the above. In particular embodiments, the anti-HLA-E antibodies comprise immunoglobulin molecules and immunologically active portions of immunoglobulin molecules. In particular embodiments, the anti-HLA-E antibodies comprise monoclonal antibodies. In particular embodiments, the anti-HLA-E antibodies comprise purified monoclonal antibodies. In particular embodiments, the anti-HLA-E antibodies comprise polyclonal antibodies. In particular embodiments, the anti-HLA-E antibodies comprise purified polyclonal antibodies. In other embodiments, the anti-HLA-E antibodies comprise Fab fragments.

Anti-HLA-E antibodies described herein can be of any subclass of IgG (e.g., IgG1, IgG2 (e.g., IgG2a and IgG2b), IgG3, IgG4) of immunoglobulin molecule. In some embodiments, the anti-HLA-E antibodies are IgG antibodies. In particular embodiments, the antibodies comprise IgG I antibodies.

Anti-HLA-E antibodies include both modified antibodies (i.e., antibodies that comprise a modified IgG (e.g., IgG1) constant domain, or FcRn-binding fragment thereof (e.g., the Fc-domain or hinge-Fc domain)) and unmodified antibodies (i.e., antibodies that do not comprise a modified IgG (e.g., IgG1) constant domain. or FcRn-binding fragment thereof (e.g., the Fc-domain or hinge-Fc domain)), that bind to HLA-E and not HLA-F and HLA-G polypeptides (e.g., heavy chain polypeptides). Techniques of making modified antibodies are well known to those skilled in the art. In some embodiments of the pharmaceutical compositions provided herein. the anti-HLA-E antibodies are modified antibodies. In some embodiments, the anti-HLA-E antibodies comprise modified loG constant domain or FcRn-binding fragments.

In some embodiments, the anti-HLA-E antibodies are modified to increase the in vivo serum half life. In some embodiments, the anti-HLA-E antibodies comprise modified IgG constant domain or FcRn-binding fragments that increase the in vivo serum half-lives of the antibodies. In some embodiments, the anti-HLA-E antibodies are attached to inert polymer molecules to prolong the in vivo serum circulation of the antibodies. In particular embodiments, the inert polymer molecules are high molecular weight polyethyleneglycols (PEGs). PEGs can be attached to the antibodies with or without a multifunctional linker either through site-specific conjugation of the PEG to the N- or C-terminus of the antibodies or via epsilon-amino groups present on lysine residues. In another embodiment, the anti-HLA-E antibodies are conjugated to albumin. The techniques are well-known in the art. See, e.g., International Publication Nos. WO 93/15199, WO 93/15200, and WO 01/77137; and European Patent No. EP 413,622, all of which are incorporated herein by reference.

In some embodiments, the anti-HLA-E antibodies are immunoreactive to the heavy chain polypeptide of HLA-E and are not immunoreactive to the heavy chain polypeptide of HLA-F or HLA-G. In some embodiments, the anti-HLA-E antibodies are immunoreactive to the heavy chain polypeptide of HLA-E and are not immunoreactive to the heavy chain polypeptide of HLA-F or HLA-G or to β2-microglobulin.

In certain embodiments, anti-HLA-E antibodies provided herein are immunoreactive to HLA-E either in native or denatured confirmation. In some embodiments, the anti-HLA-E antibodies provided herein are immunoreactive to HLA-E in native form (i.e., an HLA-E heavy chain polypeptide in native form). In other embodiments, the anti-HLA-E antibodies provided herein are immunoreactive to HLA-E in denatured form (i.e., a denatured HLA-E heavy chain polypeptide).

In some embodiments, the anti-HLA-E antibodies are also immunoreactive to one or more HLA Ia antigens. The HLA Ia loci is highly polymorphic and, therefore, there exists many alleles for HLA-A (767 alleles). HLA-B (1,178 alleles) and HLA-Cw (439 alleles). Antibodies immunoreactive to HLA-E can bind to a shared peptide (or epitope) sequences in a polypeptide encoded by a particular allele of HLA-A, HLA-B or HLA-C as determined by any method known to those skilled in the art, including, but not limited to, RIAs (radioimmunoassays), enzyme immunoassays like ELISAs (enzyme-linked immunosorbent assays), LIAs (luminescent immunoassays) and FIAs (fluorescent immunoassays), in which the antibodies, either used as primary or secondary antibodies, are labeled with radioisotopes (e.g., ¹²⁵I). fluorescent dyes (e.g., PC or FITC) or enzymes (e.g., peroxidase or alkaline phosphatase) that catalyze fluorogenic or luminogenic reactions. An HLA Ia antigen comprises an HLA heavy chain or portion of an HLA heavy chain that is bound to another HLA heavy chain to form a homodimer, or an HLA heavy chain or portion of an HLA heavy chain associated with a β2-microglobulin to form a heterodimer or an HLA heavy chain or portion of an HLA heavy chain that is free (Le., not bound to another HLA or β2-microglobulin). HLA antigens include those expressed or located on a cell surface or those occurring in soluble form in circulation or body fluids.

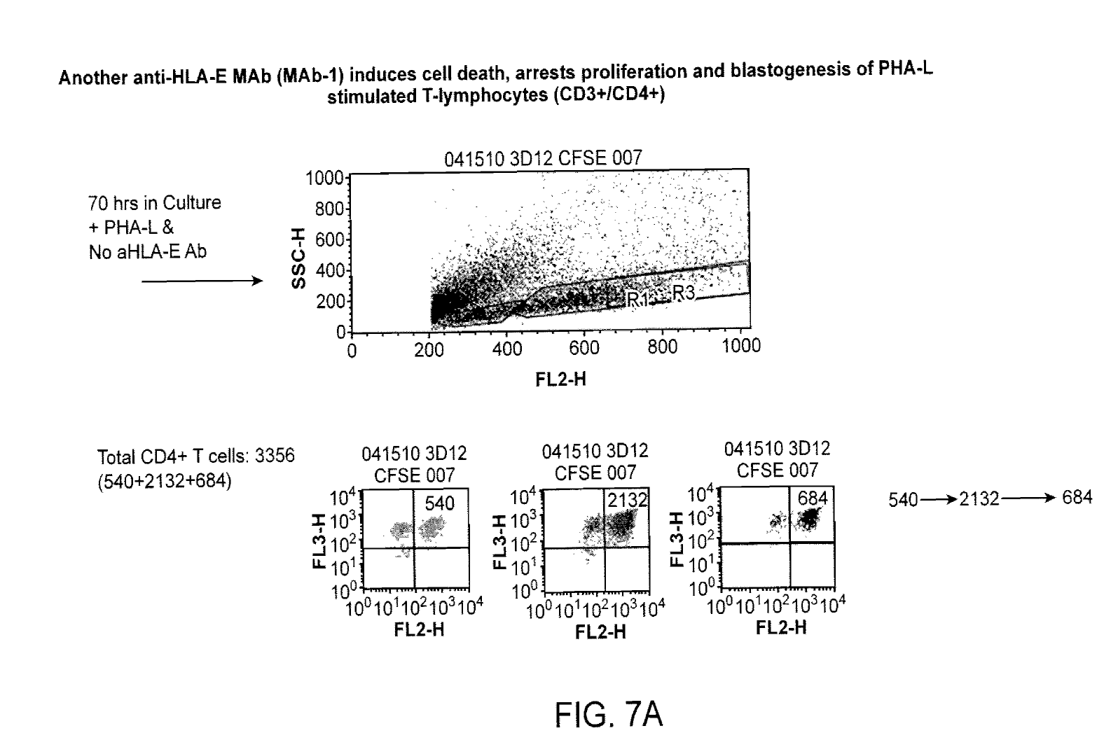

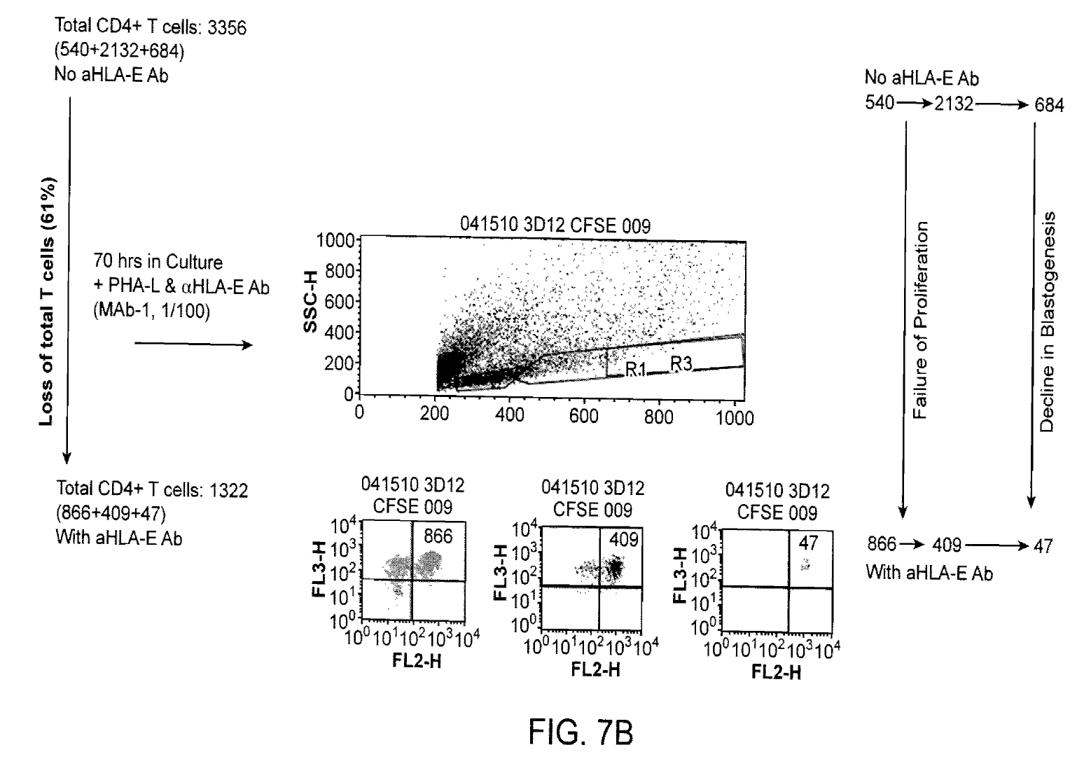

When an anti-HLA-E antibody binds an HLA-E or HLA Ia expressed on the surface of a cell, it can (1) suppress the immune activities of the cell; (2) cause death of the cell either by apoptosis or necrosis; (3) induce cytotoxicity to the cell; or (4) activate or stimulate the target cell to proliferate. For example, an anti-HLA-E antibody described herein may suppress proliferation of PHA-L activated CD4+ T-lymphocytes, activate naïve CD8+ T-cells and induce cytotoxicity in CD8+ lymphoblasts. See FIGS. 7, 8, 9E-G, 11 and 12.

When an anti-HLA-E antibody described herein binds a soluble HLA-E or HLA Ia antigen, it can block or prevent the activities of the soluble HLA antigen. For example, the anti-HLA-E antibody may prevent the soluble HLA antigen from binding to a receptor on a lymphocyte to suppress or trigger death of the lymphocyte or activate the lymphocyte as described above. Such blocking or inhibition of the soluble HLA antigen is referred to as “neutralization.” Furthermore, an anti-HLA-E antibody described herein that binds to a soluble HLA antigen in circulation or a body fluid may clear the soluble HLA antigen from the circulation or body fluid before the soluble HLA causes any drastic effect on an immune system. Without being bound to any particular theory of operation, it is believed that the therapeutic efficacy of an anti-HLA-E antibody provided herein is dependent on the ability of the anti-HLA-E antibody to bind to a particular HLA-E or HLA Ia.

In certain embodiments, the anti-HLA-E antibodies are also immunoreactive to HLA-A, HLA-B or HLA-Cw. In certain embodiments, the anti-HLA-E antibodies are also immunoreactive to at least one HLA-A. In certain embodiments, the anti-HLA-E antibodies are also immunoreactive to several HLA-B. In certain embodiments, the anti-HLA-E antibodies are also immunoreactive to several HLA-Cw . In certain embodiments, the anti-HLA-E antibodies are also immunoreactive to at least one HLA-A and more than one HLA-B and HLA-Cw. In certain embodiments, the anti-HLA-E antibodies are also immunoreactive to more than one of HLA-A, HLA-B and HLA-Cw. In certain embodiments, the anti-HLA-E antibodies are also immunoreactive to less than five HLA-A and to more than five HLA-B and HLA-Cw.

Pharmaceutical Compositions

In certain embodiments, provided herein are pharmaceutical compositions comprising antibodies in a pharmaceutically acceptable carrier. In some embodiments, the pharmaceutical composition comprises antibodies, wherein at least 70% of the antibodies are anti-HLA-E antibodies. In certain embodiments, at least 75% of the antibodies are anti-HLA-E antibodies. In certain embodiments, at least 80% of the antibodies are anti-HLA-E antibodies. In certain embodiments, at least 85% of the antibodies are anti-HLA-E antibodies. In certain embodiments, at least 90% of the antibodies are anti-HLA-E antibodies. In certain embodiments, at least 95% of the antibodies are anti-HLA-E antibodies. In certain embodiments, at least 99% of the antibodies are anti-HLA-E antibodies. In other embodiments, at least 99.5% of the antibodies are anti-HLA-E antibodies.

In some embodiments of the pharmaceutical compositions provided herein, the immunoreactivity of the anti-HLA-E antibodies can be blocked by one or more particular peptides comprising an amino acid sequence listed in Table 3 or combinations thereof.

TABLE 3
Peptide sequences of HLA-E shared and not
shared(*) by other Class Ia and Ib alleles

| | | HLA alleles | | | | | |
|---|---|---|---|---|---|---|---|
| HLA-E peptide sequences | | Class | | | Non-classical | | |
| SEQ ID | | HLA Ia | | | HLA Ib | | |
| NO: | Amino Acid Sequence | A | B | Cw | F | G | Specificity |
| 1 | ⁴⁷PRAPWMEQE⁵⁵ | 1 | 0 | 0 | 0 | 0 | A*3306 |
| 2 | ⁵⁹EYWDRETR⁶⁵ | 5 | 0 | 0 | 0 | 0 | A-restricted |
| 3 | ⁹⁰AGSHTLQW⁹⁷ | 1 | 10 | 48 | 0 | 0 | Polyspecific |
| 4 | ¹⁰⁸RFLRGYE¹²³ | 24 | 0 | 0 | 0 | 0 | A-restricted |
| 5 | ¹¹⁵QFAYDGKDY¹²³ | 1 | 194 | 75 | 0 | 0 | Polyspecific |
| 6 | ¹¹⁷AYDGKDY¹²³ | 491 | 831 | 271 | 21 | 30 | Polyspecific |
| 7 | ¹²⁶LNEDLRSWTA¹³⁵ | 239 | 210 | 261 | 0 | 30 | Polyspecific |
| 8 | ¹³⁷DTAAQI¹⁴² | 0 | 824 | 248 | 0 | 30 | Polyspecific |
| 9 | ¹³⁷DTAAQIS¹⁴³ | 0 | 52 | 4 | 0 | 30 | Polyspecific |
| 10 | ¹⁵⁷RAYLED¹⁶² | 0 | 1 | 0 | 0 | 0 | B*8201 |
| 11 | ¹⁶³TCVEWL¹⁶⁸ | 282 | 206 | 200 | 0 | 30 | Polyspecific |
| 12 | ¹⁸²EPPKTHVT¹⁹⁰ | 0 | 0 | 19 | 0 | 0 | C-restricted |
| 13 | ⁶⁵RSARDTA⁷¹ (*) | 0 | 0 | 0 | 0 | 0 | E-restricted |
| 14 | ¹⁴³SEQKSNDASE¹⁵² (*) | 0 | 0 | 0 | 0 | 0 | E-restricted |

The amino acid sequences listed in Table 3 are amino acid sequences (with the exception of two sequences: RSARDTA (SEQ ID NO: 13) and SEQKSNDASE (SEQ ID NO: 14) that were found to be shared by at least one HLA-E and one HLA Ia. Thus, while not being bound to any particular theory of operation, it is believed that in some embodiments, the immunoreactivity of the anti-HLA-E antibodies can be blocked by polypeptides having at least one of these amino acid sequences. In some embodiments of the pharmaceutical compositions provided herein, the immunoreactivity of the anti-HLA-E antibodies can be blocked by polypeptides comprising the amino acid sequence PRAPWMEQE (SEQ ID NO: 1). In some embodiments, the immunoreactivity of the anti-HLA-E antibodies can be blocked by polypeptides comprising the amino acid sequence EYWDRETR (SEQ ID NO: 2). In some embodiments, the immunoreactivity of the anti-HLA-E antibodies can be blocked by polypeptides comprising the amino acid sequence AGSHTLQW (SEQ ID NO: 3). In some embodiments, the immunoreactivity of the anti-HLA-E antibodies can be blocked by polypeptides comprising the amino acid sequence RFLRGYE (SEQ ID NO: 4). In some embodiments, the immunoreactivity of the anti-HLA-E antibodies can be blocked by polypeptides comprising the amino acid sequence QFAYDGKDY (SEQ ID NO: 5). In some embodiments. the immunoreactivity of the anti-HLA-E antibodies can be blocked by polypeptides comprising the amino acid sequence AYDGKDY (SEQ ID NO: 6). In some embodiments, the immunoreactivity of the anti-HLA-E antibodies can be blocked by polypeptides comprising the amino acid sequence LNEDLRSWTA (SEQ ID NO: 7). In some embodiments, the immunoreactivity of the anti-HLA-E antibodies can be blocked by polypeptides comprising the amino acid sequence DTAAQI (SEQ ID NO: 8). In some embodiments, the immunoreactivity of the anti-HLA-E antibodies can be blocked by polypeptides comprising the amino acid sequence DTAAQIS (SEQ ID NO: 9). In some embodiments, the immunoreactivity of the anti-HLA-E antibodies can be blocked by polypeptides comprising the amino acid sequence RAYLED (SEQ ID NO: 10). In some embodiments, the immunoreactivity of the anti-HLA-E antibodies can be blocked by polypeptides comprising the amino acid sequence TCVEWL (SEQ ID NO: 11). In some embodiments, the immunoreactivity of the anti-HLA-E antibodies can be blocked by polypeptides comprising the amino acid sequence EPPKTHVT (SEQ ID NO: 12). In some embodiments, the immunoreactivity of the anti-HLA-E antibodies can be blocked by unshared polypeptide comprising the amino acid sequence RSARDTA (SEQ ID NO: 13). In some embodiments, the immunoreactivity of the anti-HLA-E antibodies can be blocked by unshared polypeptide comprising the amino acid sequence SEQKSNDASE (SEQ ID NO: 11). In some embodiments, the immunoreactivity of the anti-HLA-E antibodies can be blocked by a polypeptide comprising the amino acid sequences QFAYDGKDY (SEQ ID NO: 5) and DTAAQI (SEQ ID NO: 8). In some embodiments, the immunoreactivity of the anti-HLA-E antibodies can be blocked by a polypeptide comprising the amino acid sequences QFAYDGKDY (SEQ ID NO: 5) and DTAAQI (SEQ ID NO: 8), wherein the sequences QFAYDGKDY and DTAAQI are discontinuous. See, e.g., Ravindranath et al., 2010,. Mol. Immunol. 47: 1121-1131. In some embodiments, the immunoreactivity of the anti-HLA-E antibodies can be blocked by polypeptides. wherein each polypeptide comprises the amino acid sequences QFAYDGKDY (SEQ ID NO: 5), LNEDLRSWTA (SEQ ID NO: 7) and DTAAQI (SEQ ID NO: 8).

Without being bound to any particular theory of operation, it is believed that the pharmaceutical compositions described herein can suppress proliferation and/or blastogenesis of naïve and/or activated T-cells in a recipient of the pharmaceutical composition. See, e.g., FIGS. 5, 7 and 8. Further, without being bound to any particular theory of operation, it is believed that the pharmaceutical compositions described herein can induce cell death of naïve and/or activated T-cells in a recipient of the pharmaceutical composition. See, e.g., FIGS. 5, 7 and 8.

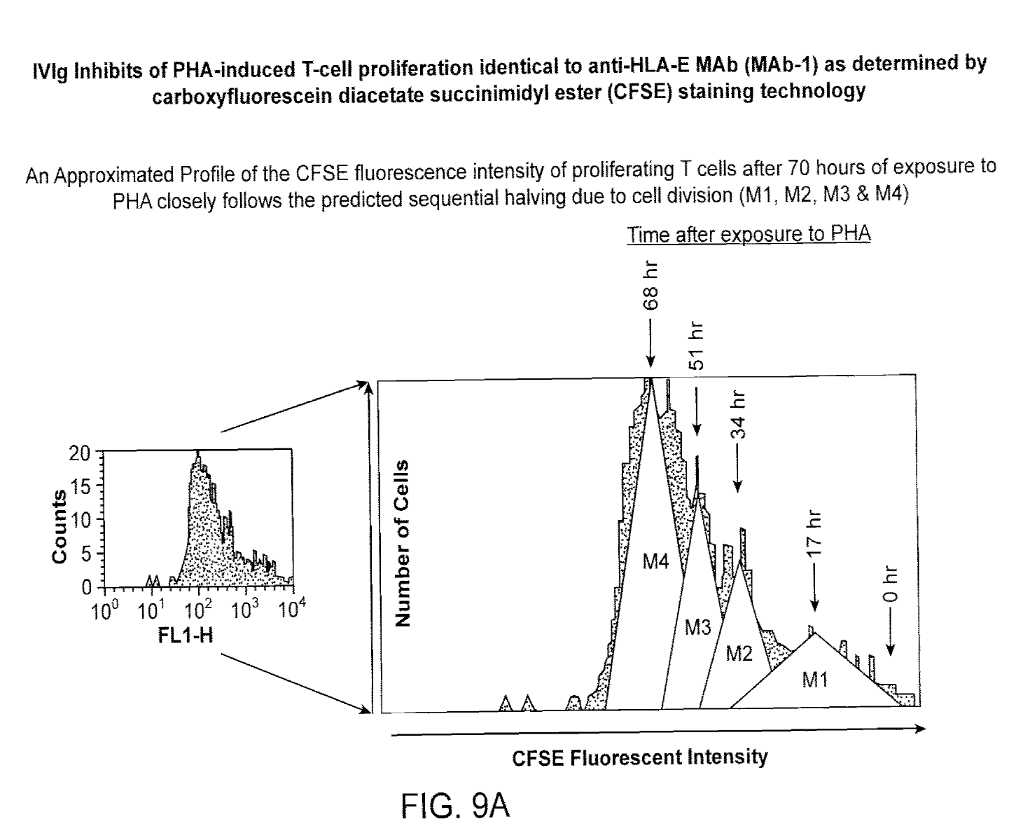

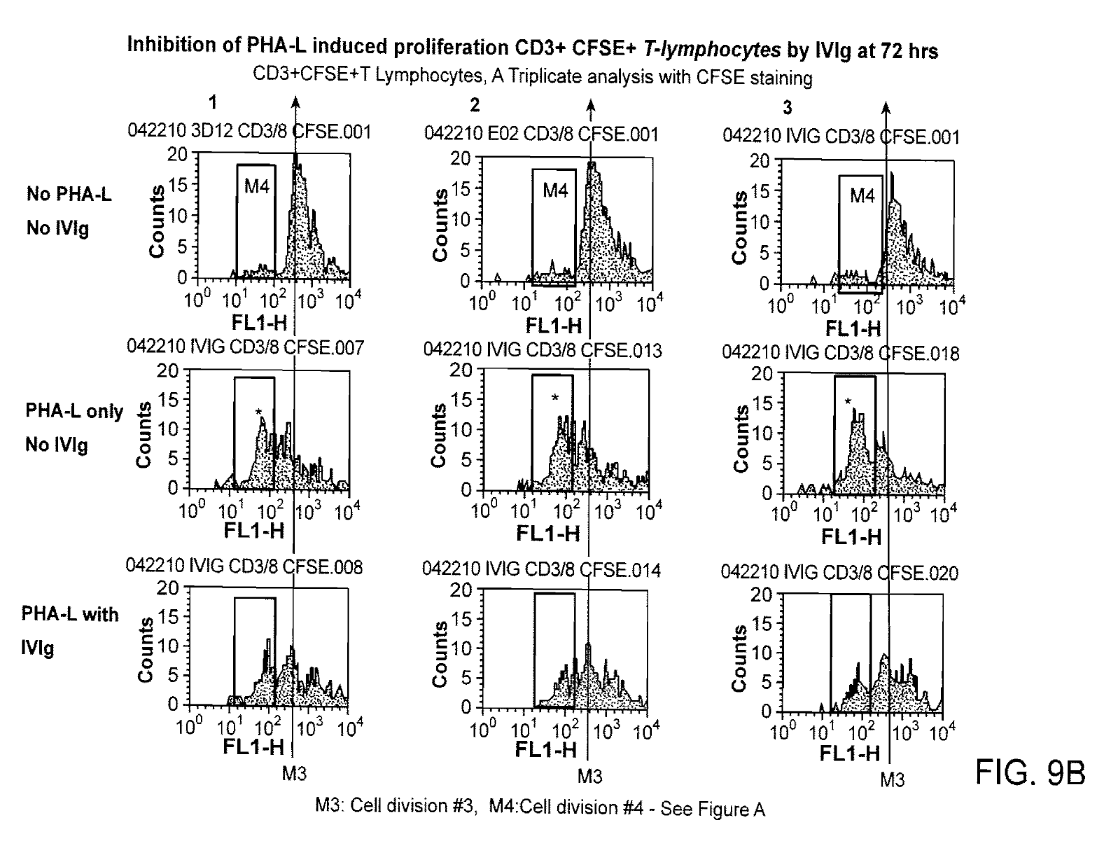

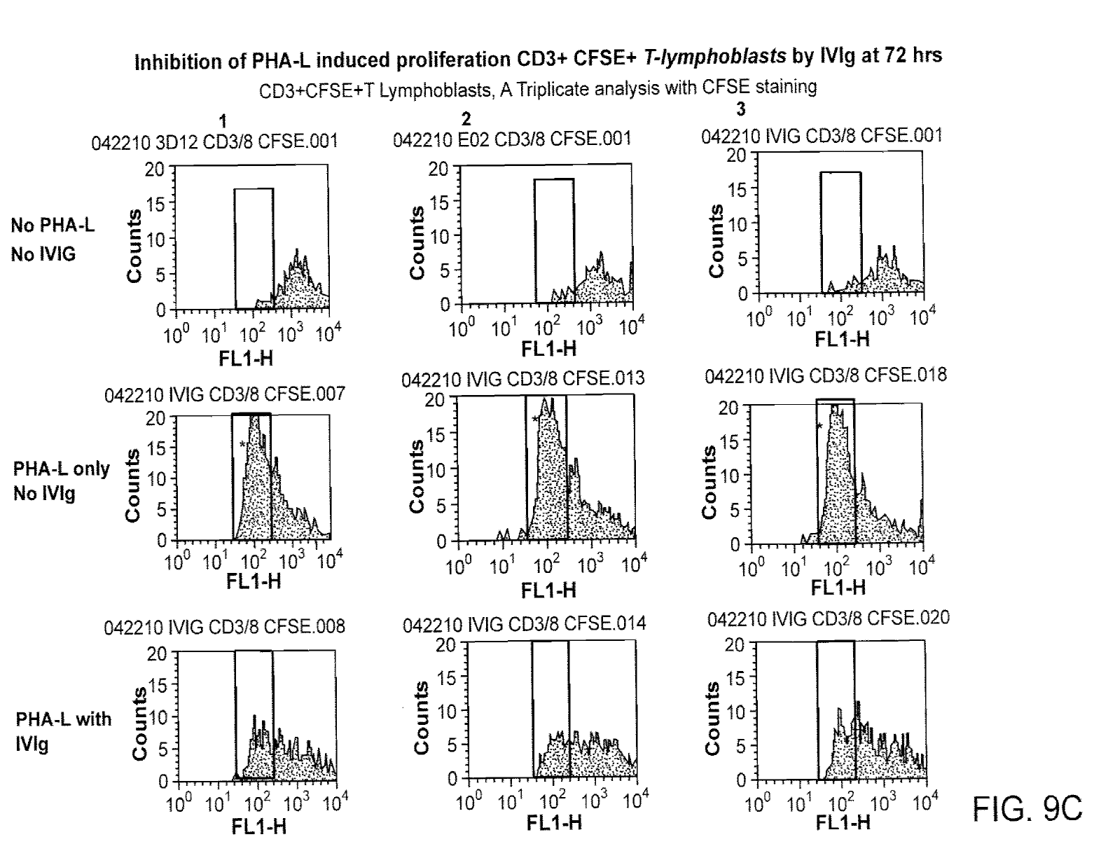

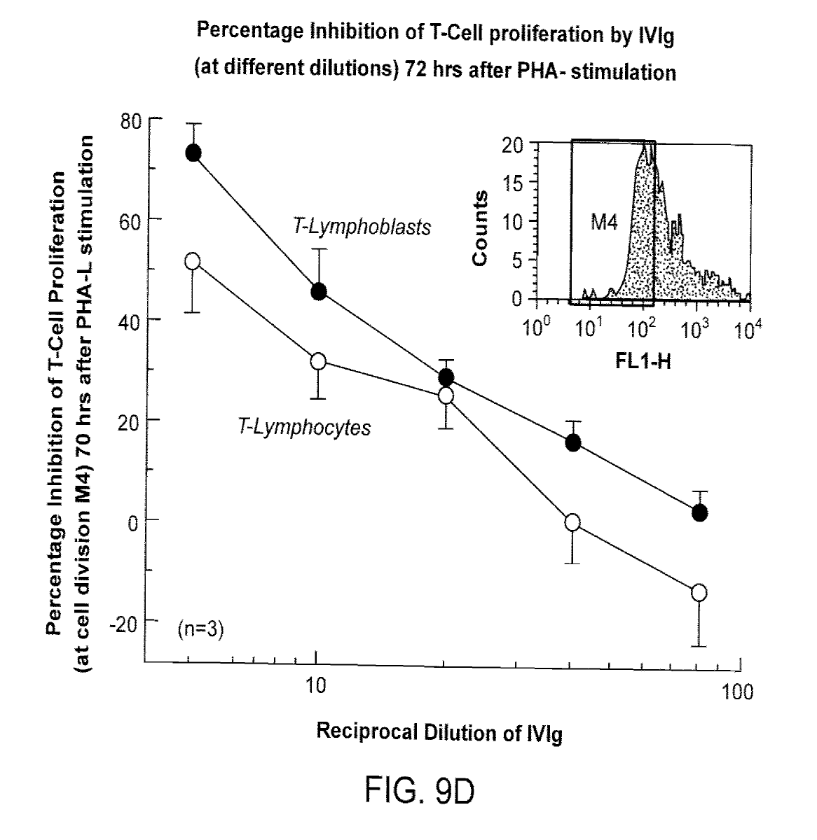

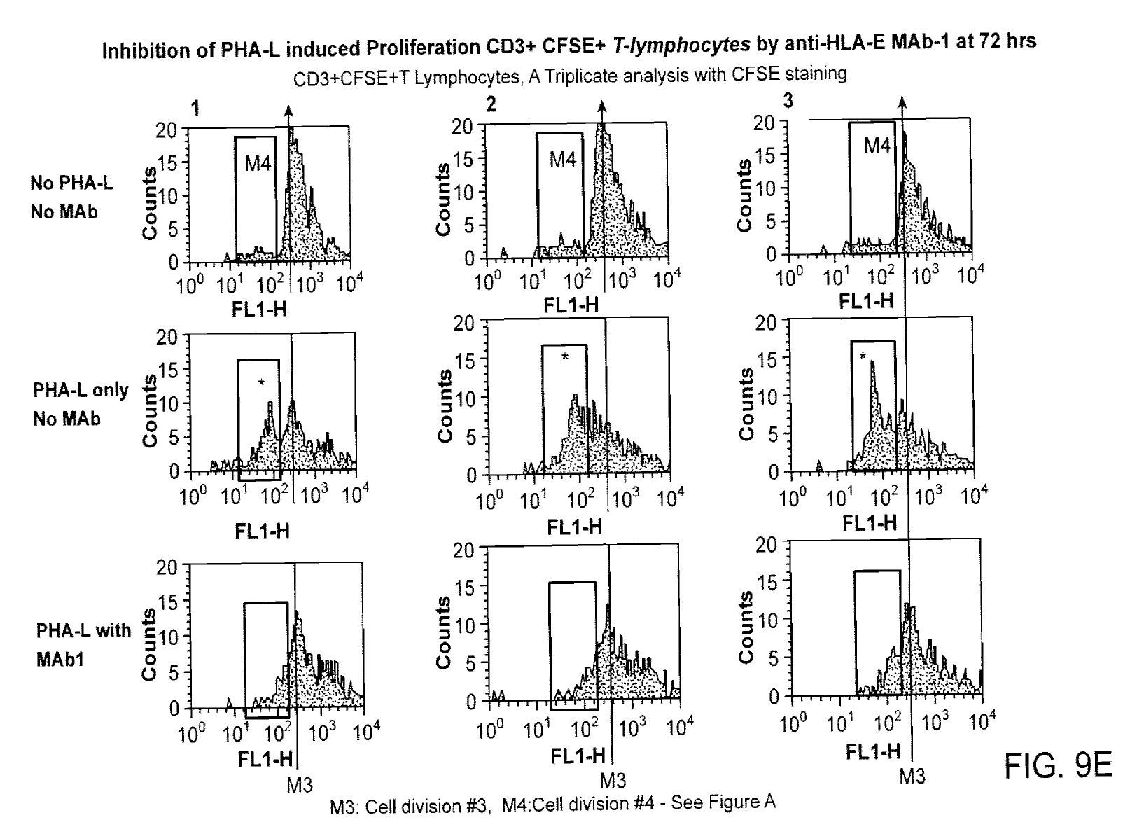

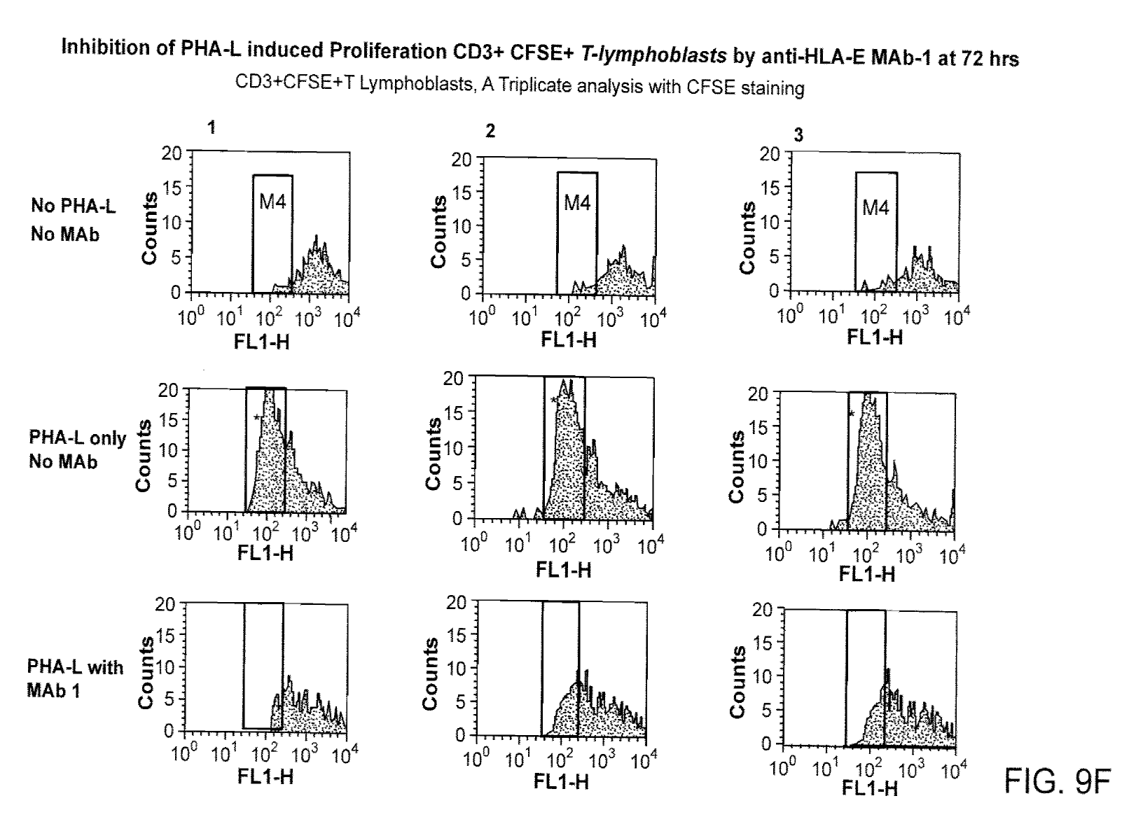

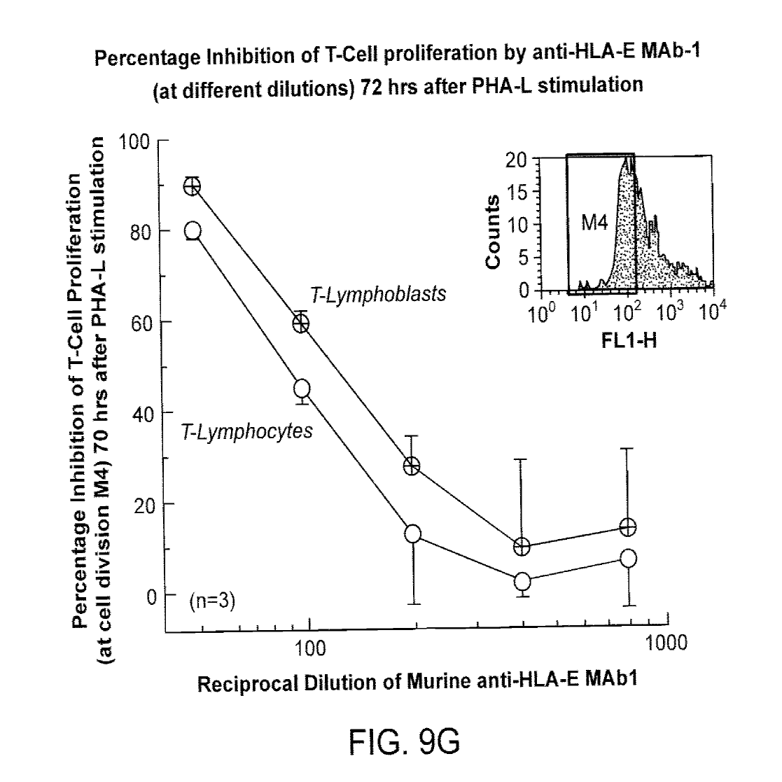

In some embodiments provided herein, the pharmaceutical composition is capable of suppressing proliferation and/or blastogenesis of naïve and/or activated T-cells in a recipient of the pharmaceutical composition. See, e.g., FIGS. 5, 7 to 14. Techniques to determine suppression of T-cell proliferation and blastogenesis are well known to those skilled in the art, including, for example, flow cytometry analysis. In certain embodiments, the pharmaceutical composition is capable of suppressing proliferation of naïve CD3+/CD4+ T-cells in a recipient of the pharmaceutical composition. In certain embodiments, the pharmaceutical composition is capable of suppressing proliferation of activated CD3+/CD4+ T-cells in a recipient of the pharmaceutical composition. In certain embodiments, the pharmaceutical composition is capable of suppressing blastogenesis of naïve CD3+/CD4+ T-cells in a recipient of the pharmaceutical composition. See, e.g., FIGS. 9F-G and 11. In certain embodiments, the pharmaceutical composition is capable of suppressing blastogenesis of activated CD3+/CD4+ T-cells in a recipient of the pharmaceutical composition. See, e.g., FIGS. 9F-G and 11.

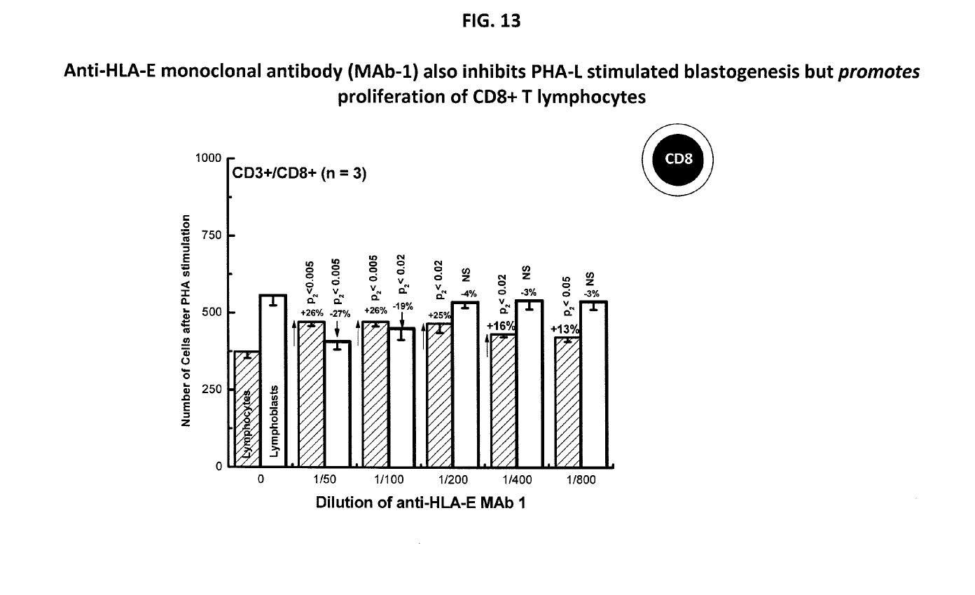

In certain embodiments, the pharmaceutical composition is capable of suppressing proliferation of naïve CD3+/CD8+ T-cells in a recipient of the pharmaceutical composition. In certain embodiments, the pharmaceutical composition is capable of suppressing proliferation of activated CD3+/CD8+ T-cells in a recipient of the pharmaceutical composition. See, e.g., FIG. 13. In certain embodiments, the pharmaceutical composition is capable of suppressing blastogenesis of naïve CD3+/CD8+ T-cells in a recipient of the pharmaceutical composition. In certain embodiments, the pharmaceutical composition is capable of suppressing blastogenesis of activated CD3+/CD8+ T-cells in a recipient of the pharmaceutical composition. See, e.g., FIG. 13.

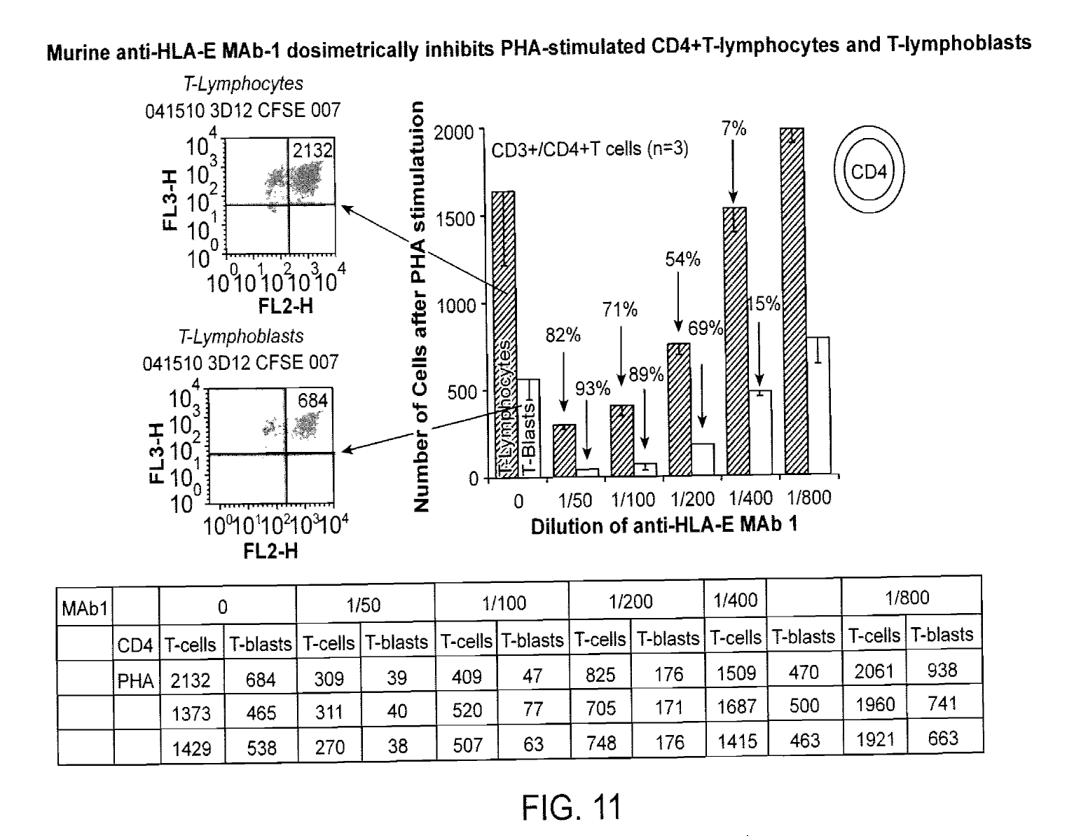

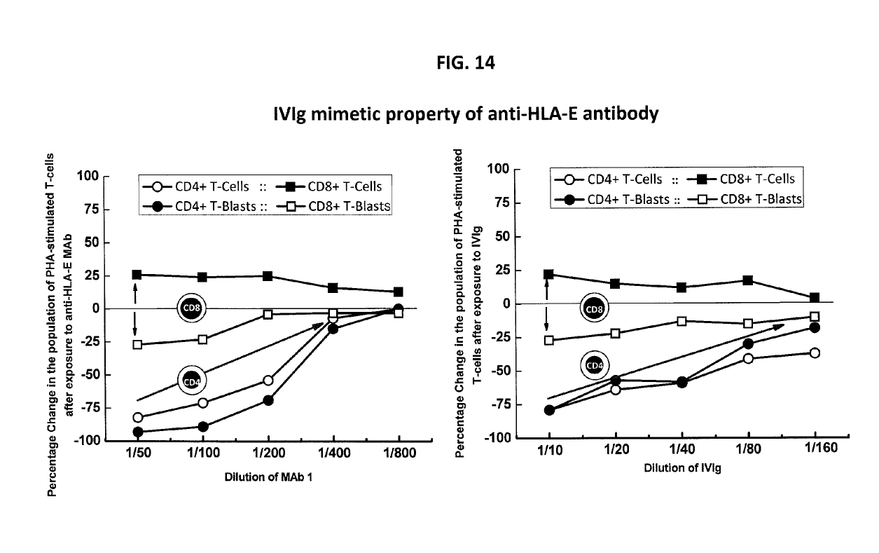

In some embodiments provided herein, the pharmaceutical composition is capable of inducing cell death of naïve and/or activated T-cells in a recipient of the pharmaceutical composition. In certain embodiments, the pharmaceutical composition is capable of inducing cell death of naïve CD3+/CD4+ T-cells. See, e.g., FIGS. 11 and 14. In certain embodiments, the pharmaceutical composition is capable of inducing cell death of activated CD3+/CD4+ T-cells. See, e.g., FIGS. 11 and 14. In certain embodiments, the pharmaceutical composition is capable of inducing cell death of naïve CD3+/CD8+ T-cells. See, e.g., FIGS. 13 and 14. In certain embodiments, the pharmaceutical composition is capable of inducing cell death of activated CD3+/CD8+ T-cells. See, e.g., FIGS. 13 and 14.

In certain embodiments, the pharmaceutical composition is capable of inducing apoptosis of naïve and/or activated CD3+/CD4+ T-cells in a recipient of the pharmaceutical composition. See, e.g., FIGS. 7, 8, 9E-G, 11 and 14. In certain embodiments, the pharmaceutical composition is capable of inducing apoptosis of naïve and/or activated CD3+/CD8+ T-cells in a recipient of the pharmaceutical composition. See, e.g., FIGS. 13 and 14. In certain embodiments, the pharmaceutical composition is capable of inducing necrosis of nave and/or activated CD3+/CD4+ T-cells in a recipient of the pharmaceutical composition. See, e.g., FIGS. 7, 8, 9E-G, 11 and 14. In certain embodiments, the pharmaceutical composition is capable of inducing necrosis of naïve and/or activated CD3+/CD8+ T-cells in a recipient of the pharmaceutical composition. See, e.g., FIGS. 13 and 14.

Without being bound to any particular theory of operation, it is believed that the pharmaceutical compositions described herein can suppress formation of T-cell dependent anti-HLA antibodies in a recipient. In certain embodiments, the pharmaceutical composition is capable of suppressing formation of T-cell dependent anti-HLA-A antibodies. In certain embodiments, the pharmaceutical composition is capable of suppressing formation of T-cell dependent anti-HLA-B antibodies. In certain embodiments, the pharmaceutical composition is capable of suppressing formation of T-cell dependent anti-HLA-Cw antibodies. In certain embodiments, the pharmaceutical composition is capable of suppressing formation of T-cell dependent anti-HLA-E antibodies. In certain embodiments, the pharmaceutical composition is capable of suppressing formation of T-cell dependent anti-HLA-F antibodies. In certain embodiments, the pharmaceutical composition is capable of suppressing formation of T-cell dependent anti-HLA-G antibodies.

Without being bound to any particular theory of operation, it is believed that the pharmaceutical compositions described herein can block or neutralize the proinflammatory or adverse effects caused by a soluble HLA antigen by interfering with the ability of the soluble HLA antigen to bind to a lymphocyte bound receptor in a body fluid or circulation. In certain embodiments, the anti-HLA-E antibodies are capable of blocking or neutralizing the proinflammatory or adverse effects caused by a soluble HLA antigen by interfering with the ability of the soluble HLA to bind to a lymphocyte bound receptor in a body fluid or circulation.

Without being bound to any particular theory of operation, it is believed that the pharmaceutical compositions described herein can clear soluble HLA heavy chains from circulation. In some embodiments. the pharmaceutical composition is capable of clearing HLA heavy chains from circulation.

In some embodiment of the pharmaceutical compositions provided herein, the anti-HLA-E antibodies are immunoreactive to HLA la antigens similar to a commercial preparation of IVIg. See, e.g., FIG. 4. HLA-E antibodies that are immunoreactive to HLA Ia antigens similar to IVIg bind to a percentage of the same HLA la antigens as IVIg as determined by any method known to those skilled in the art. A comparison of the binding of HLA Ia antigens by IVIg and the pharmaceutical compositions provided herein can be performed using any technique known to those skilled in the art, including, but not limited to, enzyme-linked immunosorbent assays (ELISAs). Commercial sources of IVIg are available, for example, from Baxter, Bayer, Centeon and Novartis. In some embodiments of the pharmaceutical composition provided herein, the anti-HLA-E antibodies are immunoreactive to at least 50% of the same FILA Ia antigens as a commercial preparation of IVIg. See, e.g., FIG. 4. In certain embodiments, the anti-HLA-E antibodies are immunoreactive to at least 60% of the same HLA Ia antigens as a commercial preparation of IVIg. See, e.g., FIG. 4. In certain embodiments, the anti-HLA-E antibodies are immunoreactive to at least 70% of the same HLA la antigens as a commercial preparation of IVIg. In certain embodiments, the anti-HLA-E antibodies are immunoreactive to at least 75% of the same HLA Ia antigens as a commercial preparation of IVIg. In certain embodiments, the anti-HLA-E antibodies are immunoreactive to at least 80% of the same HLA Ia antigens as a commercial preparation of IVIg. In certain embodiments, the anti-HLA-E antibodies are immunoreactive to at least 85% of the same HLA Ia antigens as a commercial preparation of IVIg. In certain embodiments, the anti-HLA-E antibodies are immunoreactive to at least 90% of the same HLA Ia antigens as a commercial preparation of IVIg. In certain embodiments, the anti-HLA-E antibodies are immunoreactive to at least 95% of the same HLA Ia antigens as a commercial preparation of IVIg. In certain embodiments, the anti-HLA-E antibodies are immunoreactive to at least 99% of the same HLA Ia antigens as a commercial preparation of IVIg.

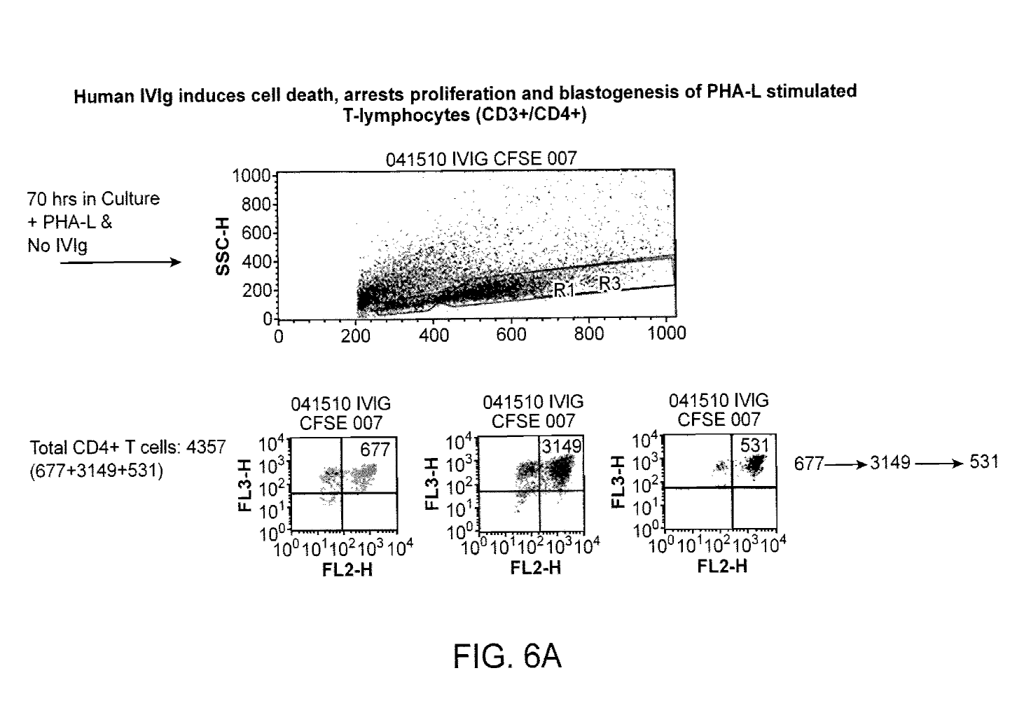

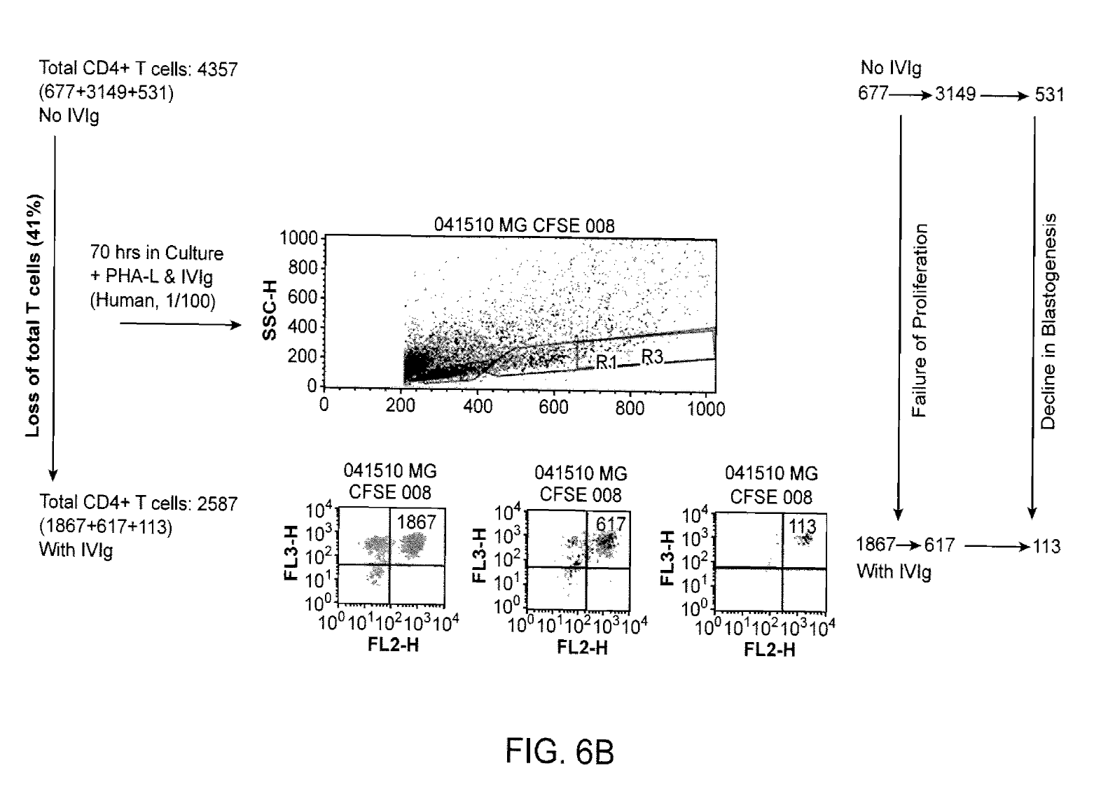

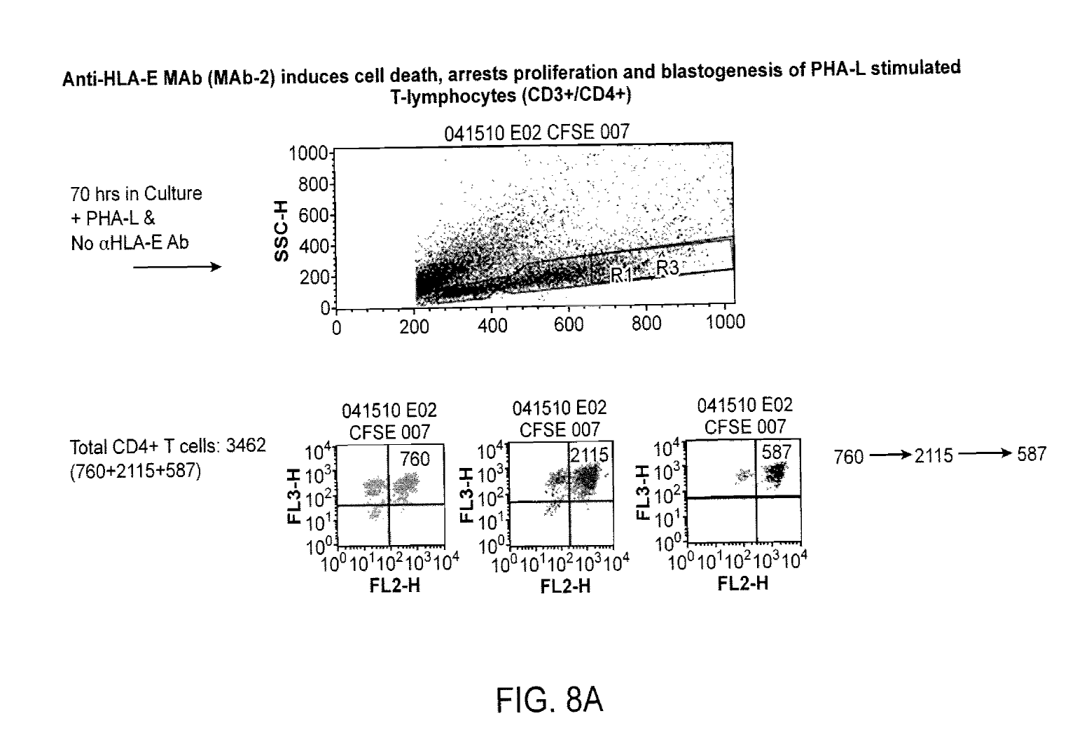

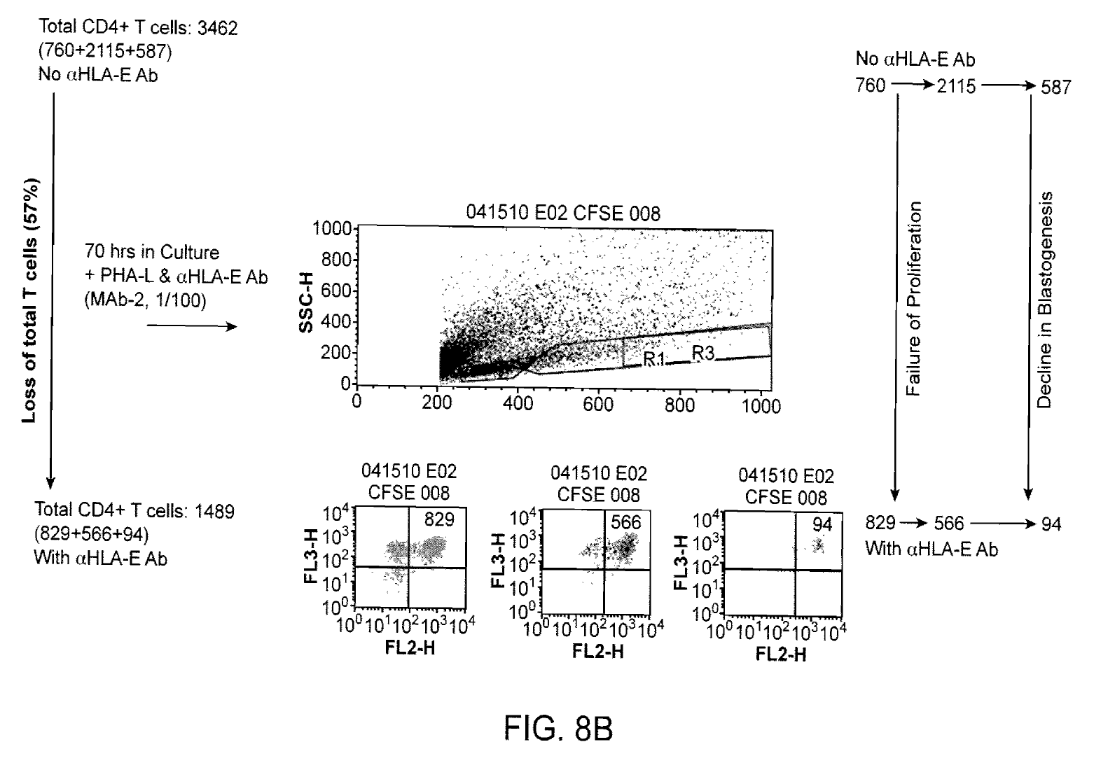

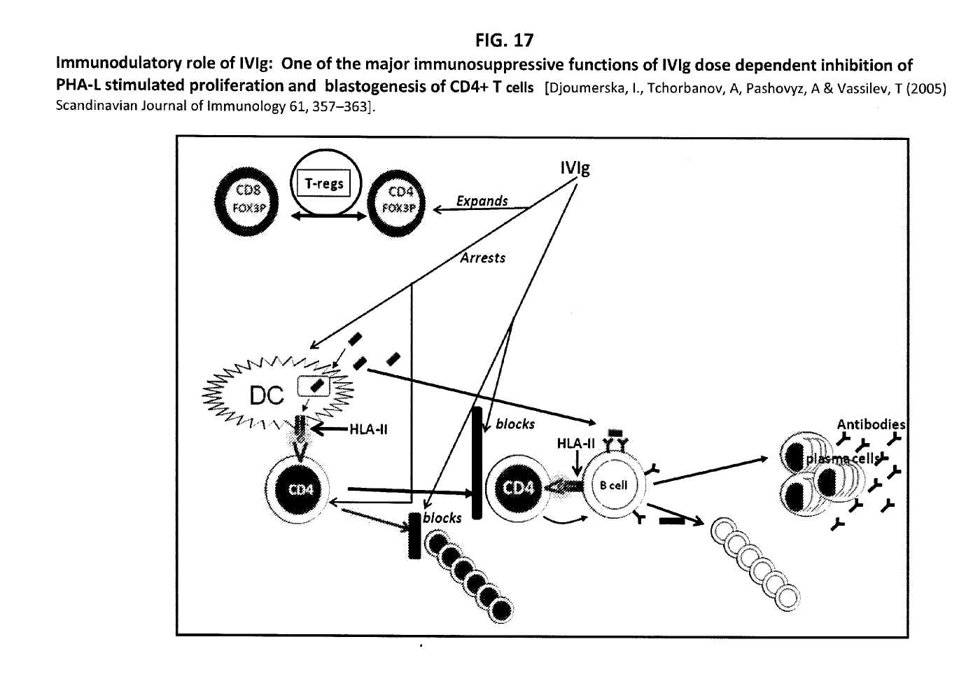

In some embodiments of the pharmaceutical composition, the anti-HLA-E antibodies have immunomodulatory activity comparable to a commercial preparation of IVIg (for example, compare FIGS. 7 and 8 with FIG. 6, or FIG. 9E-G with FIG. 9 B-D; FIG. 14). Commercial preparations of IVIg are thought to provide immunomodulatory effects within a recipient. These immunomodulatory activities of IVIg are thought to include, but are not limited to, modulation of T-cell, B-cell and dendritic cell growth, expansion or proliferation, downregulation of expression of MI-IC class II molecules, inhibition of expression of CD80/CD86 molecules, suppression of dendritic cell-mediated activation and proliferation of alloreactive T-cells, induction of apoptosis of T-cells, suppression of the expansion of autoreactive B-cells, inhibition of complement activation, and enhancement of clearance of endogenous pathogenic autoantibodies. See FIG. 17. Without being bound to any particular theory of operation, it is believed that a pharmaceutical composition comprising anti-HLA-E antibodies has at least one or more of the same immunomodulatory activities as compared to a commercial preparation of IVIg. The immunomodulatory activities described above can be measured by any technique known to those skilled in the art. In some embodiments, the anti-HLA-E antibodies modulate T-cell growth, expansion and/or proliferation comparable to a commercial preparation of IVIg (for example, compare FIGS. 7 and 8 with FIG. 6, or FIG 3. 9E-H with FIG. 9 C-E; FIG. 14). In some embodiments, the anti-HLA-E antibodies modulate B-cell growth, expansion and/or proliferation similar to a commercial preparation of IVIg. In some embodiments, the anti-HLA-E antibodies modulate dendritic cell growth, expansion and/or proliferation comparable to a commercial preparation of IVIg. In some embodiments, the anti-HLA-E antibodies modulate downregulation of expression of MHC class II molecules comparable to a commercial preparation of IVIg. In some embodiments, the anti-HLA-E antibodies modulate inhibition of expression of CD80/CD86 molecules comparable to a commercial preparation of IVIg. In some embodiments, the anti-HLA-E antibodies modulate suppression of dendritic cell-mediated activation and/or proliferation of alloreactive T-cells comparable to a commercial preparation of IVIg. In some embodiments, the anti-HLA-E antibodies modulate suppression of the expansion of autoreactive B-cells comparable to a commercial preparation of IVIg. In some embodiments, the anti-HLA-E antibodies modulate suppression of the inhibition of complement activation comparable to a commercial preparation of IVIg. In some embodiments, the anti-HLA-E antibodies modulate the enhancement of clearance of endogenous pathogenic autoantibodies comparable to a commercial preparation of IVIg.

In some embodiments, the pharmaceutical composition provided herein is therapeutically effective for the treatment of one or more inflammatory diseases or conditions treatable by a commercial preparation of IVIg. Without being bound to any particular theory of operation, it is believed that a pharmaceutical composition comprising anti-HLA-E antibodies can mimic the immunomodulatory effects of whole IVIg. Thus, it is believed that in some embodiments, the pharmaceutical compositions provided herein are therapeutically effective for the treatment of one or more inflammatory diseases or conditions treatable by IVIg. A pharmaceutical composition that is therapeutically effective for the treatment of a particular disease or condition reduces the severity, the duration and/or the number of sy mptoms associated with that disease or condition. Inflammatory diseases and conditions treatable by commercial preparations of IVIg include, but are not limited to: Kawasaki disease, immune-mediated thrombocytopenia, primary immunodeficiencies, hematopoietic stem cell transplantation, chronic B-cell lymphocytic leukemia, pediatric HIV type 1 infection, aplastic anemia, pure red cell aplasia, Diamond-Blackfan anemia, autoimmune hemolytic anemia, hemolytic disease of the newborn, acquired factor I inhibitors, acquired von Willebrand disease, immune-mediated neutropenia, refractoriness to platelet transfusion, neonatal alloimmune thrombocytopenia, posttransfusion purpura, thrombotic thrombocytopenic purpura/hemolytic uremic syndrome, hemolytic transfusion reaction, hemophagocytic syndrome thrombocytopenia, acute lymphoblastic leukemia, multiple myeloma, human T-cell lymphotrophic virus-1-myelopathy, nephritic syndrome, membranous nephropathy, nephrotic syndrome, acute renal failure, epilepsy, chronic inflammatory, demyelinating polyneuropathy, Guillain-Barre syndrome, myasthenia gravis, Lambert-Eaton myasthenic syndrome, multifocal motor neuropathy, multiple sclerosis, Wegener granulomatosis, amyotrophic lateral sclerosis, lower motor neuron syndrome, acute disseminated encephalomyelitis, paraneoplastic cerebellar degeneration, paraproteinemic neuropathy, polyneuropathy, progressive lumbosacral plexopathy, lyme radiculoneuritis, endotoxemia of pregnanacy, parvovirus infection, streptococcal toxic shock syndrome, rheumatoid arthritis, systemic lupus erythematosus, dermatomyositis, polymyositis, inclusion-body myositis, autoimmune blistering dermatosis, cardiomyopathy, acute cardiomyopathy, euthyroid ophthalmopathy, uveitis, recurrent otitis media, asthma, cystic fibrosis, Behcet syndrome. chronic fatigue syndrome, congenital heart block, diabetes mellitus, acute idiopathic dysautonomia, opsoclonus-myoclonus, Rasmussen syndrome. Reiter syndrome, Vogt-Koyanagi-Harada syndrome, trauma and burns. In some embodiments, the pharmaceutical composition is therapeutically effective for the treatment of one or more of the aforementioned inflammatory diseases or conditions treatable by a commercial preparation of IVIg.

Pharmaceutically Acceptable Carriers

The pharmaceutical compositions provided herein also comprise a pharmaceutically acceptable carrier. The carrier can be a diluent, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in E. W. Martin, 1990, Remington's Pharmaceutical Sciences, Mack Publishing Co.

Formulations

In some embodiments, the pharmaceutical composition is provided in a form suitable for administration to a human subject. In some embodiments, the pharmaceutical composition will contain a prophylactically or therapeutically effective amount of the anti-HLA-E antibody together with a suitable amount of carrier so as to provide the form for proper administration to the patient. The formulation should suit the mode of administration.

In some embodiments, the pharmaceutical composition is provided in a form suitable for intravenous administration. Typically, compositions suitable for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous administration.

In particular embodiments, the pharmaceutical composition is suitable for subcutaneous administration. In particular embodiments, the pharmaceutical composition is suitable for intramuscular administration.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection. an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, the anti-HLA-E antibody is supplied as a water free concentrate. In some embodiments. the antibody is supplied as a dry sterile lyophilized powder at a unit dosage of at least 0.5 mg. at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 ma, or at least 75 mg.

In another embodiment, the pharmaceutical composition is supplied in liquid form. In some embodiments, the pharmaceutical composition is provided in liquid form and is substantially free of surfactants and/or inorganic salts. In some embodiments, the antibody is supplied as in liquid form at a unit dosage of at least 0.1 mg/ml, at least 0.5 mg/ml, at least 1 mg/ml, at least 2.5 mg/ml, at least 3 mg/ml, at least 5 mg/ml, at least 8 mg/ml, at least 10 mg/ml, at least 15 mg/ml, at least 25 mg/ml, at least 30 mg/ml, or at least 60 mg/ml.

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Methods for Treatment of Diseases

In another aspect provided herein are methods of preventing, managing, treating and/or ameliorating various diseases. the method comprising administering to a human subject a therapeutically effective amount of any one of the pharmaceutical compositions provided herein.

Studies described herein show that anti-HLA-E antibodies can recapitulate the immunosuppressive effects of whole IVIg. Anti-HLA-E antibodies in commercial preparations of IVIG account for the immunomodulatory activity of IVIG. Thus, while not intending to be bound by any particular theory of operation, it is believe that pharmaceutical compositions comprising anti-EILA-E antibodies can be used as immunodulatory agents in preventing, managing, treating and/or ameliorating various diseases and conditions treatable by IVIg.

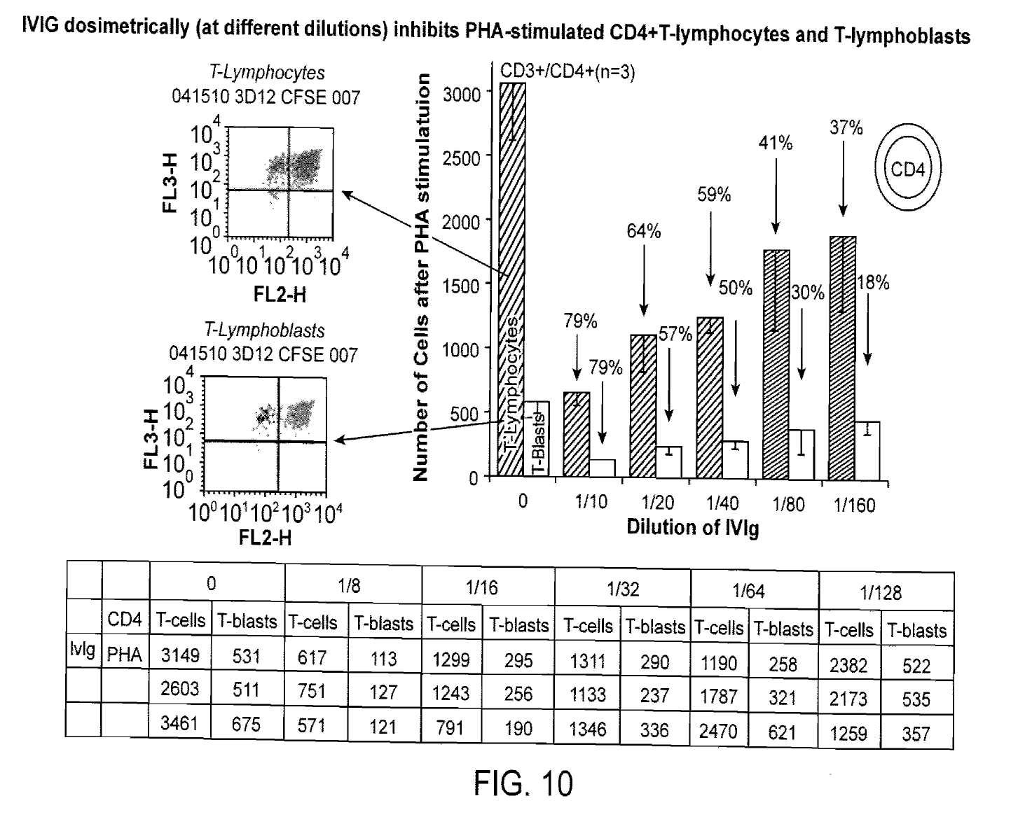

A therapeutically effective amount of the pharmaceutical composition is an amount that is required to reduce the severity, the duration and/or the symptoms of a particular disease or condition. The amount of a pharmaceutical composition that will be therapeutically effective in the prevention, management, treatment and/or amelioration of a particular disease can be determined by standard clinical techniques. The precise amount of the pharmaceutical composition to be administered with depend, in part, on the route of administration, the seriousness of the particular disease or condition, and should be decided according to the judgment of the practitioner and each human patient's circumstances. Effective amounts may be extrapolated from dose-response curves derived from preclinical protocols either in vitro using T-cells from patients as illustrated in FIGS. 10 and 11 or using in vivo animal (e.g., Wistar or Lewis rat or different strains of mice used for different diseases, or Cynomolgous monkey) test systems.

In some embodiments, the effective amount of an antibody of the pharmaceutical composition provided herein is between about 0.025 mg/kg and about 1000 mg/kg body weight of a human subject. In certain embodiments, the pharmaceutical composition is administered to a human subject at an amount of about 1000 mg/kg body weight or less, about 950 mg/kg body weight or less, about 900 mg/kg body weight or less, about 850 mg/kg body weight or less, about 800 mg/kg body weight or less, about 750 mg/kg body weight or less, about 700 mg/kg body weight or less, about 650 mg/kg body weight or less, about 600 mg/kg body weight or less, about 550 mg/kg body weight or less, about 500 mg/kg body weight or less, about 450 mg/kg body weight or less, about 400 mg/kg body weight or less, about 350 mg/kg body weight or less, about 300 mg/kg body weight or less, about 250 mg/kg body weight or less, about 200 mg/kg body weight or less, about 150 mg/kg body weight or less, about 100 mg/kg body weight or less, about 95 mg/kg body weight or less, about 90 mg/kg body weight or less. about 85 mg/kg body weight or less. about 80 mg/kg body weight or less, about 75 mg/kg body weight or less. about 70 mg/kg body weight or less. or about 65 mg/kg body weight or less.

In some embodiments, the effective amount of an antibody of the pharmaceutical composition provided herein is between about 0.025 mg/kg and about 60 mg/kg body weight of a human subject. In some embodiments, the effective amount of an antibody of the pharmaceutical composition provided herein is about 0.025 mg/kg or less, about 0.05 mg/kg or less, about 0.10 mg/kg or less, about 0.20 mg/kg or less, about 0.40 mg/kg or less, about 0.80 mg/kg or less, about 1.0 mg/kg or less, about 1.5 mg/kg or less, about 3 mg/kg or less, about 5 mg/kg or less, about 10 mg/kg or less, about 15 mg/kg or less, about 20 mg/kg or less, about 25 mgikg or less, about 30 mg/kg or less, about 35 mg/kg or less, about 40 mg/kg or less, about 45 mg/kg or less, about 50 mg/kg or about 60 mg/kg or less.

In some embodiments, the method further comprises coadministrating to the human subject one or more immunosuppressive agents with the pharmaceutical composition. Examples of immunosuppressive agents that can be coadministered with the pharmaceutical composition include, but are not limited to corticosteroids, vitamin D3. azathioprine, prednisone, cylcosporin, cyclophosphamide, OKT3, FK506, mycophenolic acid or the morpholinethylester thereof, 15-deoxyspergualin, rapamycin. mizoribine, misoprostol, anti-interleukin-1 receptor antibodies, an anti-lymphocyte globulin, Velcade, Bortesomib, inhibitors of plasma cells and antibody production, NFKB, MERK, Akt, Jun pathway inhibitors, and phytonutrients or plant chemical nutrients, such as carotenoids (alpha-carotene, beta-carotene, lycopene, lutein, zeaxanthin, and cryptoxanthin), capsaisin, coumarins, flavanoids, flavonolignans, xilibinin or mixture of silymarin (silibinin A and B, isosibilinin A and B, silicristin, silidianin), ellagic acid, isoflavones, isothiocyanates, lignans, polyphenols (e.g., epicatechins-EC, epicatechin gallate-ECG, epigallocatechin-EGC, epigallocatechin gallate, EGCG, oxidized qu(nonoids, curcuminoids, curcumin), saponins and phytosterols.

The pharmaceutical composition of the method can be administered using any method known to those skilled in the art. For example, the pharmaceutical composition can be administered intramuscularly, intradermally, intraperitoneally, intravenously, subcutaneously administration, or any combination thereof. In some embodiments, the pharmaceutical composition is administered subcutaneously. In some embodiments, the composition is administered intravenously. In some embodiments, the composition is administered intramuscularly.

Allograft Rejection

In one aspect provided herein, is a method of preventing, managing. treating and/or ameliorating an allograft rejection, the method comprising administering to a human subject a therapeutically effective amount of any one of the pharmaceutical compositions provided herein.

Rejection of donated grafts (e.g., organs, tissue, or cells) by a transplant recipient can be caused by anti-HLA antibodies directed against the HLA-antigens of the donor in the sera of the recipient. IVIg has been used as an immunodulatory agent in the prevention, management and treatment of allograft rejections. See, e.g., Glotz et al., 2004, Transpl Int 17: 1-8. As shown in the studies described herein, anti-HLA-E antibodies in commercial preparations of IVIg alone can recapitulate immunomodulatory effects of whole IVIg. Thus, without being bound to any particular theory of operation. it is believed that pharmaceutical compositions comprising the immunodulatory component of IVIg, anti-HLA-E antibodies, are also useful in the prevention, management, treatment and amelioration of allograft rejections.

In some embodiments, the allograft is an organ. In some embodiments, the allograft is a heart, kidney or lung. In particular embodiments, the allograft is a heart. In particular embodiments, the allograft is a kidney. In other embodiments, the allograft is a lung. In some embodiments, the allograft is a tissue. In other embodiments, the graft is a plurality of cells. In some embodiments, the allograft is a plurality of bone marrow cells. In some embodiments the allograft is a plurality of blood cells.

In some embodiments, the pharmaceutical composition is administered to the human subject prior to transplantation. In some embodiments, the pharmaceutical composition is administered to the human subject at a therapeutically effective amount of 0.1 to about 1000 mg/kg body weight. In some embodiments, the pharmaceutical composition is administered to the human subject at a therapeutically effective amount of 1 to about 500 mg/kg body weight.

Methods of Treatment of Other Diseases

In another aspect provided herein, is a method of managing, treating and/or ameliorating a disease or condition selected from the aforementioned diseases or conditions listed in Section 2. In some embodiments, is a method of managing, treating and/or ameliorating a disease or condition selected from the group consisting of: Kawasaki disease, immune-mediated thrombocytopenia, a primary immunodeficiency, hematopoietic stem cell transplantation. chronic B-cell lymphocytic leukemia, pediatric HIV type 1 infection, a hematological disease, nephropathy, neuropathy, a bacterial infection, a viral infection, an autoimmune disease that is not vasculitis, cardiomyopathy, an eye or ear disease, a lung disease, recurring pregnancy loss, Behcet syndrome, chronic fatigue syndrome, congenital heart block, diabetes mellitus, acute idiopathic dysautonomia, opsoclonus-myoclonus, Rasmussen syndrome, Reiter syndrome, or Vogt-Koyanagi-Harada syndrome, the method comprising administering to a human subject a therapeutically effective amount of any one of the pharmaceutical compositions provided herein.

IVIg has been shown to be a useful immunodulatory agent in the prevention. management, treatment and amelioration of the disease conditions listed in Section 2. Thus, compositions comprising the immunodulatory component of IVIg, anti-HLA-E antibodies, are thought to also be useful in the prevention, management, treatment and amelioration of such conditions.

In one embodiment of the method, the disease or condition is Kawasaki disease. In another embodiment, the disease or condition is immune-mediated thrombocytopenia. In another embodiment, the disease or condition is a primary immunodeficiency. In another embodiment, the disease or condition is hematopoietic stem cell transplantation. In another embodiment, the disease or condition is chronic B-cell lymphocytic leukemia. In another embodiment, the disease or condition is pediatric HIV type 1 infection.

In some embodiments, the disease or condition is a hematological disease. In certain embodiments, the hematological disease is aplastic anemia, pure red cell aplasia, Diamond-Blackfan anemia, autoimmune hemolytic anemia, hemolytic disease of the newborn, acquired factor I inhibitors, acquired von Willebrand disease, immune-mediated neutropenia, refractoriness to platelet transfusion, neonatal alloimmune thrombocytopenia, posttransfusion purpura, thrombotic thrombocytopenic purpura/hemolytic uremic syndrome, hemolytic transfusion reaction, hemophagocytic syndrome thrombocytopenia, acute lymphoblastic leukemia, multiple myeloma, or human T-cell lymphotrophic virus-1-myelopathy.

In some embodiments, the disease or condition is nephropathy. In some embodiments, the nephropathy is nephritic syndrome, membranous nephropathy, nephrotic syndrome, or acute renal failure.

In some embodiments, the disease or condition is neuropathy. In some embodiments, the neuropathy is epilepsy. chronic inflammatory demyelinating polyneuropathy and Guillain-BarreSyndrome, myasthenia gravis, Lambert-Eaton myasthenic syndrome, multifocal motor neuropathy, multiple sclerosis, Wegener granulomatosis, Amyotrophic lateral sclerosis, lower motor neuron syndrome, acute disseminated encephalomyelitis, paraneoplastic cerebellar degeneration, paraproteinemic neuropathy, polyneuropathy, or progressive lumbosacral plexopathy.

In some embodiments, the disease or condition is an infection. In certain embodiments, the infection is an HIV infection, lyme radiculoneuritis, endotoxemia of pregnancy, a paroN, irus infection or streptococcal toxic shock syndrome.

In some embodiments, the disease or condition is an autoimmune disease that is not vasculitis. In certain embodiments, the autoimmune disease is rheumatoid arthritis, systemic lupus erythematosus, dermatomyositis, polymyositis, inclusion-body myositis, or autoimmune blistering dermatosis.

In some embodiments, the disease or condition is cardiomyopathy. In particular embodiments, the cardiomyopathy is acute cardiomyopathy.

In some embodiments, the disease or condition an eye or ear disease. In particular embodiments, the eye or ear disease is euthyroid ophthalmopathy, uveitis, or recurrent otitis media.

In some embodiments, the condition is a lung disease. In specific embodiments, the lung disease is asthma or cystic fibrosis.

### EXAMPLES

The following examples are presented to further document the supporting evidences and aspects of the compositions and describe the methods provided herein. Example 1 provides evidence showing that IgG antibodies constituting IVIg have remarkable capability and very high or potent affinity for HLA-E heavy chains. Example 2 shows IVIg from two different commercial sources have immunoreactivity to HLA Ia. Example 3 provides evidence showing that the immunoreactivity of IVIg to HLA-E and HLA Ia is lost after adsorbing IVIg to Affi-Gel conjugated with HLA-E. Example 4 shows that anti-HLA-E monoclonal antibodies (MAb-1 and MAb-2) are not immunoreactive to HLA-F and HLA-G, but are immunoreactive to HLA-class Ia alleles. Example 5 depicts the activation of T-lymphocytes using a lectin Phytohemagglutinin (PHA-L), which is capable of stimulating human T-lymphocytes and inducing blastogenesis. Example 6 demonstrates that IVIg induced cell death, proliferation arrest and suppression of blastogenesis of PHA-L stimulated T-lymphocytes (CD3+/CD4+). Example 7 demonstrates that anti-HLA-E MAb-2 induced cell death, proliferation arrest and suppression of blastogenesis of PHA-L stimulated T-lymphocytes (CD3+/CD4+). Example 8 demonstrates that anti-HLA-E MAb (MAb-1) induced cell death, proliferation arrest and suppression of blastogenesis of PHA-L stimulated T-lymphocytes (CD3+/CD4+). Example 9 demonstrates that IVIg inhibition of PHA-L induced T-cell proliferation is identical to anti-HLA-E MAb-1. In this assay system, carboxufluorescein diacetate succinimidyl ester (CFSE) staining technology is used. Example 10 provides a dosimetric analysis of the effects of IVIg on PHA-L stimulated CD3+/CD8+ blastogenesis and proliferation of CD8+ T-Iymphoblasts. Example 11 provides a dosimetric analysis of the effects of anti-HLA-E MAb-1 on PHA-L stimulated CD3+/CD8+ blastogenesis and proliferation of CD8+ T-Iymphoblasts.

### Example 1

### Determination of Potential anti-HLA-E Reactivity of IgG Antibodies in IVIg

This example demonstrates that IgG immunoreactive to HLA-E is present in IVIg. Multiplex Luminex®-based immunoassay were used To detect the presence of Abs that react to HLA-E in IVIg. IVIg was obtained from two sources: (1) IVIGlob® EX, VHB Life Sciences Ltd., India; and (2) GamaSTAN™ S/D, TALECRIS, USA. IVIg was serially diluted, starting from a 1/2 dilution and ending in a 1/512 dilution with PBS (pH 7.2). Using dual-laser flow cytometric principles of Luminex® xMAP® multiplex technology, the single Ag (allele) assays were carried out for data acquisition and quantitative estimation of the level of HLA-E Abs. The Luminex® assays utilize microbeads on which HLA-E heavy chains have been covalently bonded (xMap® assays). Three kinds of beads were used: (1) negative control beads that do not contain any proteins; (2) positive control beads coated with human Immunoglobulin (Ig), most commonly IgG; and (3) experimental beads coated with HLA-E heavy chain. The recombinant HLA-E heavy chain was attached to 5.6 μ polystyrene microspheres by a process of simple chemical coupling, the microspheres internally dyed at One Lambda with red and infrared flurophores, using different intensities of two dyes (xMAP® microsphere number #005). Recombinant HLA-E folded heavy chain (10 mg/ml in MES buffer) was purchased from the core facility at the Immune Monitoring Lab., Fred Hutchinson Cancer Research Center, University of Washington, Seattle, Wash. Data generated with Luminex Multiplex Flow Cytometry (LABScan® 100) was analyzed using computer software. PE-conjugated anti-human IgG Abs were used for immunolocalization of the Ab bound to Ags coated on to the microbeads. The reporter fluorophore intensity was then measured in a specialized flow cytometer together with the microbead identifiers, and the fluorescence measurement was classified by bead identifier. Florescence intensity from a sample of 90 or more beads was collected. The Trimmed Mean is obtained by trimming a percent off the high and low ends of a distribution and finding the mean of the remaining distribution.

FIGS. 1A and 1B document the presence of IgG immunoreactive to HLA-E in IVIg. The levels of the antibody were high as evidenced at different dilutions. The values are expressed as mean fluorescent intensity (MFI). The MFI values increased from 1/2 to 1/32 dilution for IVIg from IVIGlob® EX (FIG. 1A) and from 1/2 to 1/8 dilution for IVIg from GamaSTAN™ S/D (FIG. 1B). Such increases signify the aggregation of IgG immunoreactive to HLA-E at high concentration and also indicates the high titer of anti-HLA-E IgG antibodies in the IVIg preparations.

### Example 2

### Determination of the Presence of Potential anti-HLA Ia-reactivity of IVIg Obtained from Two Different Commercial Sources

This example demonstrates that two commercial sources of IVIg are immunoreactive to HLA Ia. To detect the presence of Abs that are immunoreactive to HLA la alleles in IVIg, a multiplex Luminex®-based immunoassay was used. IVIg (IVIGlob® EX, VHB Life Sciences Ltd. India; GamaSTAN™ S/D, TALECRIS, Talecris Biotherapeutics, Inc., USA) was serially diluted starting from 1/2 dilution and ending in 1/512 dilution with PBS (pH 7.2). Using dual-laser flow cytometric principles of Luminex® xMAP® multiplex technology, the single Ag (allele) assays were carried out for data acquisition and quantitative estimation of the level of HLA-E Abs. The Luminex® assays utilize microbeads on which individual HLA Ags have been covalently bonded (xMap® assays). XMap® microbeads contain two reporter fluorophores that are proportionally varied to identify them as one of 100 possible bead identifiers. The LABScreen® (One Lambda, Canoga Park, Calif.) consists of a panel of color-coded microspheres (SAB, coated with single Ag HLA alleles) to identify Ab specificities. The array of HLA Ags representing various alleles on the beads are listed at the One Lambda website under Ab detection products/LABScreen® Single Ag Product sheet/HLA Ia combi-LS1A04-Lot 002 or LS1A04-Lot 005 Worksheet Rev-1. The SAB products in LS1A04 include 31 HLA-A, 50 HLA-B and 16 HLA-C alleles. It should be noted that not all existing HLA Ia alleles are represented in the beads analyzed.

Three kinds of microspheres or beads were used: (1) negative control beads that do not contain any proteins; (2) positive control beads coated with human Immunoglobulin (Ig), most commonly IgG; and (3) experimental beads coated with HLA-E or HLA Ia alleles. The recombinant HLA antigens were attached to 5.6 μ polystyrene microspheres by a process of simple chemical coupling, the microspheres internally dyed at One Lambda with red and infrared flurophores, using different intensities of two dyes (xMAP® microsphere number #005). Data generated with Lumeinex® Multiplex Flow Cytometry (LABScan® 100) were analyzed using computer software. PE-conjugated anti-human IgG Abs were used for immunolocalization of the Ab bound to Ags coated onto the microbeads. The reporter fluorophore intensity was then measured in a specialized flow cytometer together with the microbead identifiers, and the fluorescence measurement was classified by bead identifier. Florescence intensity from a sample of 90 or more beads was collected. The Trimmed Mean was obtained by trimming a percent off the high and low ends of a distribution and finding the mean of the remaining distribution. The legend for colored boxes are given with FIG. 2A and B.

FIGS. 2A and 2B demonstrate the presence of Abs immunoreactive to HLA Ia in two commercial sources of IVIg. The immunoreactivity of IVIg to HLA Ia shown in FIGS. 2A and 2B is comparable to that of anti-HLA-E IgG reported in FIG. 4. Human HLA class Ia antigens belonging to Cw* alleles seems to be well recognized by IVIg. Even at high dilutions, IVIg recognizes HLA-Cw* alleles. This is true for both the commercial preparations. Both HLA-E reactivity and C-alleleic reactivity of IVIg can be used to compare and standardize the potency of IVIg from different commercial sources. Both FILA-E and C-alleleic reactivity of IVIg can also be used for quality control and quality assurance of IVIg.

### Example 3

### Loss of both HLA-E and HLA Ia Reactivity of IVIg After Adsorption of IVIg to Affi-Gel Conjugated with HILA-E

This example demonstrates that HLA Ia reactivity of IVIg is due to the presence of HLA-E antibodies in IVIg. HLA-E heavy chain (6 mg) was dialyzed overnight at 4° C. against sodium bicarbonate buffer (pH 8.5) to remove Urea and DTT. For conjugating HLA-E to Affi-Gel 10, Affi-Gel 10 was washed with distilled water and sodium bicarbonate buffer for 20 minutes. After removing supernatant, HLA-E (6 mg) in 1 ml of buffer was mixed with 500 μl of the Affi-Gel 10 suspension (338 til) suspension. The mixture was kept on an inverting rotator for overnight in a refrigerator. The tube was taken out and centrifuged at 600 g for 5 minutes. The supernatant was recovered and the gel was washed three times in distilled water and twice with carbonate buffer (Elution Buffer). After removing the supernatant completely, 100 μl of IVIg (1/128 dilution) was added to the gel and mixed well. The HLA-E coupled Affi-Gel-10 and IVIg (1/128dilution) mixture was placed on an inverter for 1 hour. In the meantime, 100 μl of 1/128 diluted IVIg was further serially diluted (1/128, 1/256, 1/512 and 1/1024 dilutions, to a total volume of 50 μl). IVIg adsorbed to HLA-E gel (or control Affi-Gel 10 without HLA-E) was recovered and designated Eluate # 1a and # 1b. Eluate # 1 was also serially diluted as 1/128, 1/256, 1/512 and 1/1024 dilutions. The entire sets were tested against HLA-E beads and HLA Ia beads.

IVIg used for this specific experiment came from the same batch as the original, but had been stored in aliquots in the refrigerator for six months. Consequently. the IVIg used in the experiment had reduced potency in binding to HLA but it did bind 1/4^{th} of the original. The MFI of anti-FILA-E reactivity was >18.000 but the aliquot was 4,500.

As shown in FIGS. 3A-C, both IVIg immunoreactivity to HLA-E and HLA Ia are lost after adsorbing IVIg to HLA-E conjugated Affi-Gel. The data provides evidence that IVIg immunoreactivity to HLA Ia is due to the presence of HLA-E antibodies in IVIg.

### Example 4

### Anti-HLA-E Monoclonal Antibodies (MAb-1 and MAb-2) are Non-Reactive to HLA-F and HLA-G, but Reactive with HLA-class Ia Alleles

This example demonstrates that anti-HLA-E monoclonal antibodies (MAb-1 and MAb-2) are not immunoreactive to HLA-F and HLA-G, but are immunoreactive to HLA-class Ia alleles.

A multiplex Luminex®-based immunoassay was used to determine the HLA Ia immunoreactivity of two HLA-E specific (i.e., immunoreactive to HLA-E and not immunoreactive to other EILA Ib molecules, namely, HLA-F and HLA-G) murine monoclonal antibodies (MAb-1 and MAb-2) against HLA-E and HLA-A, HLA-B, HLA-Cw, HLA-F and HLA-G. Anti-HLA-E MAbs were diluted 1/100, 1/200 and 1/400 with PBS (pH 7.2). Using dual-laser flow cytometric principles of Lum M Luminex® xAP® multiplex technology, the single Ag (allele) assays were carried out for data acquisition and quantitative (Mean Florescent Intensity or MFI) estimation of the level of HLA-E Abs. The Luminex® assays utilize microbeads on which individual HLA Ags (HLA-E and HLA Ia antigens) have been covalently bonded (xMap® assays). XMap® microbeads contain two reporter fluorophores that are proportionally varied to identify them as one of 100 possible bead identifiers. The LABScreen® (One Lambda, Canoga Park, Calif.) consists of a panel of color-coded microspheres (SAB, coated with single Ag HLA alleles) to identify Ab specificities. The array of HLA Ags representing various alleles on the beads are listed at the One Lambda website under Ab detection products/LABScreeng Single Ag Product sheet/HLA Ia combi-LSIA04-Lot 002 Worksheet Rev-1. The SAB products in LS1A04 include 31 HLA-A, 50 HLA-Bund 16 HLA-C alleles. It should be noted that not all existing HLA Ia alleles are represented in the beads analyzed.

Three kinds of beads were used: (1) negative control beads that do not contain any proteins; (2) positive control beads coated with human Immunoglobulin (Ig), most commonly IgG; and (3) experimental beads coated with HLA-E or HLA Ia alleles. The recombinant HLA antigens were attached to 5.6 μ polystyrene microspheres by a process of simple chemical coupling, the microspheres internally dyed at One Lambda with red and infrared flurophores, using different intensities of two dyes (xMAP® microsphere number #005). Data generated with Luminex® Multiplex Flow Cytometry (LABScan® 100) were analyzed using computer software. PE-conjugated anti-Human IgG Abs were used for the immunolocalization of the Ab bound to Ags coated on to the microbeads. The reporter fluorophore intensity w as then measured in a specialized flow cytometer together with the microbead identifiers, and the fluorescence measurement was classified by bead identifier. Florescence intensity from a sample of 90 or more beads was collected. The Trimmed Mean was obtained by trimming a percent off the high and low ends of a distribution and finding the mean of the remaining distribution.

FIG. 4 summarizes the immunoreactivity of MAb-1 and MAb-2 monoclonal antibodies for HLA Ia. The tainted (bluish) HLA Ia alleles signify common alleles reacted by both the monoclonal antibodies. It is evident that immunoreactivity to HLA-E accompanies immunoreactive to HLA Ia as evidenced from the affinity of two different sources of anti-HLA-E monoclonal antibodies. As seen in FIG. 4, there are differences in recognition of some of the HLA-Ia alleles between the two antibodies. This could be due to peptide sequences recognized or not recognized in addition to recognizing the peptide sequences (epitopes) shared with HLA-E, namely ¹¹⁵QFAYDGKDY^{I23} (SEQ ID NO: 5) and ¹³⁷DTAAQI¹⁴² (SEQ ID NO: 8). It should be noted that MAb-1 recognizes ¹²⁶LNEDRSWTA^{I35}(SEQ ID NO: 7), an epitope not recognized by MAb-2. See Ravindranath et al., 2010, Mol. Immunol. 47: 1121-1131; Ravindranath et al., 2010, J. Immunol. 185: 1935-1948; Ravindranath, et al., 2011, Mol. Immunol. 48: 423-430.

### Example 5

### Activation of T-Lymphocytes using a Lectin Phytohemagglutinin (PHA-L), which is Capable of Stimulating Human T-Lymphocytes and Inducing Blastogenesis

This example depicts the activation of T-lymphocytes using a lectin Phytohemagglutinin (PHA-L). which is capable of stimulating human T-lymphoeytes and inducing blastogenesis. PHA-L stimulated T-lymphocytes were used to test the ability of IVIg and the claimed antibodies provided herein to induce cell death, proliferation arrest and suppression of blastogenesis.

Events occurring 70 hrs after PHA-L stimulation of T-lymphocytes (CD3+/CD4+) were assessed using whole blood (20 ml) drawn from a healthy donors into Acid Citrate Dextrose (ACD) tubes. Whole blood (15 ml) was pipetted into 25 ml of PBS (without calcium or magnesium) in a 50 ml conical centrifuge tube and underlayed with Ficoll-Hypaque (10 ml) at room temperature. After centrifugation (20 min. at 800 g (2000 rpm in H-1000 rotor), 20° C.)), the plasma-platelet-containing supernatant was aspirated from above the interface band. The interface band, which includes the lymphocytes, was then aspirated with <5 ml of fluid and transferred to a new 50 ml centrifuge tube. combining the bands from 2 to 3 Ficoll-Hypaque gradients. PBS was then added to the combined interface bands to a total volume of 50 ml and centrifuged (10 min. at 600g (1500 rpm in H-1000 rotor), 20° C.). The supernatants were aspirated and the pellet in each tube was combined and resuspended in 10 ml of PBS at RT. PBS was then added to a volume of 50 ml and the mixture was centrifuged (15 min. 300 g (750 rpm in H-1000 rotor), 20° C.). The lymphocyte pellet was resuspended in PBS (1 ml) at RT and the viable cells were counted. The cells were then distributed equally among three Fisher tubes with PBS and centrifuged (1 min. at 1000 g). The supernatant was discarded and the pellet was re-suspended and mixed well with 0.8 ml of Lympho-Kwik® T. The mixture was incubated (20 min. at 37° C. or RT) in a water bath or heat block with occasional mixing by inverting capped tube. PBS (0.2 ml) was then layered over the cell preparation and centrifuged (2 min. at 2000 g). The pellet was resuspended in PBS and centrifuged (1 min. at 1000g). Washing was repeated once and each pellet was resuspended in 0.8 ml of the following Lympho-Kwik® T Prep. The entire mixing, incubation, centrifugation and resuspension of pellet was repeated. In the final step, the pellet was resuspended in A1M-V medium +1% HEPES at a final concentration of 5×10⁷ cells/ml. An aliquot was tested for purity of T-cel Is using CD3 monoclonal antibody in flow cytometry. The cells were labeled with CFSE. The quantity of cells labeled was 10⁵ to 10⁶ cells per ml 10% heparinized donor plasma added. Two microliters of 5 mM CFSE per milliliter cells (final 10 μM) was added into a tube that was ≧6× the volume of cells. The cells were incubated (15 min. at RT or for 10 min. at 37° C.). The staining was quenched by adding 5 vol ice-cold AIM-V medium (+1% HEPES buffer. with 10% heparinized plasma from donor) and the cells were incubated on ice for 5 min. The cells were washed three times in the culture medium to ensure that CFSE bound to protein in the supernatant was removed, preventing any subsequent uptake into bystander cells.

The in vitro cell culture assays were set up in 96 well tissue culture plates. Purified PHA-L were added to specific wells at a concentrations of 1.12 μg/ml. The final cell concentration was 2×10⁵ cells/well. Negative and positive controls were run in triplicates. For negative controls, 10 μl it of CFSE labeled cells (2×10⁵cells) were added to wells containing 190 μl of AIM-V. For positive controls, 10 μl of CFSE labeled cells (2×10⁵ cells in 100 μl/well) were added to wells containing 90 μl of PHA-L in AIM-V and 100 μl of AIM-V. One of the three profiles of the controls is presented in FIG. 5.

In FIG. 5. the three upper and lower quadrants of flow cytometric profiles refer to different populations of T-cells (CD3+/CD4+). Two monoclonal antibodies were used: (1) CD3 MAb is indicated by the Y or vertical axis: and (2) CD4 MAb is indicated by the X or horizontal axis. Upper left quadrant: CD3 positive cells; lower left quadrant: CD3 negative cells; upper right quadrant: CD3 positive and CD4 positive T-Iymphocytes; lower right quadrant: CD3 negative and CD4 negative cells. Cells stained green, in the left most quadrants were CD3+/CD4+ primordial nave T-cells. Cells stained red in the middle quadrants were CD3+/CD4+ activated T-cells. Cells stained pink in the right quadrants were CD3+/CD4+ T-lymphoblasts. Lymphoblasts were identified by the size of the cells which results in migration of the cells towards left or upper side, indicative of the increased size and possibly granulation. In comparing PHA negative (after 70 hrs) quadrants (upper three) with PHA positive (after 70 hrs) quadrants (lower three), one may notice an increase in the cell populations of the middle quadrants (red) and right most quadrants (pink). The increase in number of pink cells (93 to 684) signify the increase in lymphoblasts after exposure to PHA for 70 hrs. Similarly, there was an increase in the number of red cells in the middle quadrants (417 to 2132), which is indicative of increase in activated T-Iymphocytes, all of which are CD4 positive T-lymphocytes. This experiment was done in triplicate and FIG. 5 is representative of one sample. FIGS. 5A and B depict the same experiment. While FIG. 5A clarifies the details and cell types involved, FIG. 5B provides and details the outcome of the experiment after exposing the lymphocytes to PHA for 70 hours.

### Example 6

### IVIg Induced Cell Death, Proliferation Arrest and Suppression of Blastogenesis of PHA-L Stimulated T-Lymphocytes (CD3+/CD4+)

This example demonstrates that IVIg can induce cell death, proliferation arrest and suppression of blastogenesis of PHA-L stimulated T-lymphocytes (CD3+/CD4+).

To determine the ability of IVIg to induce cell death, proliferation arrest and suppression of blastogenesis of PHA-L stimulated T-lymphocytes (CD3+/CD4+), an in vitro cell culture assay, similar to the one described in Example 5, was used.

CD3+ cells were isolated from whole blood, washed and labeled with CSFE using the protocol described in Example 5.

The in vitro cell culture assays were set up in 96 well tissue culture plates. Purified PHA-L was added to specific wells at a concentrations of 1.12 μg/ml. The final cell concentration w as 2×10⁵ cells/well. Negative and positive controls were run in triplicates. For PHA-L without IVIg control, 10 μl of CFSE labeled cells (2×10⁵ cells in 100 μl/well) were added to 90 μl of PHA-L in AIM-V and 100 μl of AIM-V. For PHA-L with IVIg, 10 μl of CFSE labeled cells (2×10⁵ cells in 100 μl/well) were added to 90 μl of PHA-L in AIM-V and 100 μl AIM-V containing 1/100 dilution of IVIg. One of the three profiles of the controls is presented in FIG. 6.

In FIG. 6, the three upper and lower quadrants of flow cytometric profiles refer to different populations of T-cells (CD3+/CD4+). Two monoclonal antibodies were used: (1) CD3 MAb is indicated by the Y or vertical axis; and (2) CD4 MAb is indicated by the X or horizontal axis. Upper left quadrant: CD3 positive cells; lower left quadrant: CD3 negative cells; upper right quadrant: CD3 positive and CD4 positive T-Iymphocytes; lower right quadrant; CD3 negative and CD4 negative cells. Cells that are stained green in the left most quadrants are CD3+/CD4+ primordial naïve T-cells. Cells stained red in the middle quadrants are CD3+/CD4+ activated T-cells. The right quadrants refer to CD3+/CD4+ T-lymphoblasts. Lymphoblasts were identified by the size of the cells, which results in migration of the cells towards left or upper side, indicative of the increased size and possibly granulation. In comparing PHA without IVIg- quadrants (upper three) with PHA-with IVIg-quadrants, one may notice a decrease in the cell populations in the middle quadrants (red) and right most quadrants (pink) after IVIg was added. The decrease in number of pink cells (531 to 113) signify the decrease in CD3+/CD4+ lymphoblasts in the presence of IVIg even after exposure to PHA for 70 hrs. A similar decrease is seen in the middle quadrants (red) (3149 to 617) indicates a decrease in the number of activated T-Iymphocytes. The total number of CD3+/CD4+ T-lymphocytes decreased from 4357 to 2587 in the presence of IVIg. The loss of red cells in the middle quadrants signifies death of CD4+ T-cells, while loss of cells in the pink quadrant signifies arrest in blastogenesis of CD4+ T cells. The results indicate that Wig is capable of suppressing T-cell proliferation and is capable of causing cell death of CD4+ lymphocytes. Both characteristics signify the immunosuppressive nature of IVIg. This experiment was done in triplicate and FIG. 6 is representative of one sample.

To determine the dosimetric effects of IVIg induced suppression of PHA-stimulated CD4+ T-Iymphocytes and lymphoblasts, a similar experiment was performed using different dilutions of IVIg (0 dilution, 1/10, 1/20, 1/40, 1/80 and 1/160 dilution). Three values were obtained for each dilution. The mean and standard deviation was determined. FIG. 10 illustrates the percentage change in CD4+ T-Iymphocytes and lymphoblasts at each dilution of IVIg. As shown in FIG. 10, IVIg dosimetrically inhibits PHA-stimulated CD4+ T-lymphoctes and lymphoblasts.

### Example 7

### Anti-HLA-E MAb-1 Induced Cell Death, Proliferation Arrest and Suppression of Blastogenesis of PHA-L Stimulated T-lyvmphocytes (CD3+/CD4+).

This example demonstrates that anti-HLA-E MAb-1 induced cell death, proliferation arrest and suppression of blastogenesis of PHA-L stimulated T-lymphocytes (CD3+/CD4+).

To determine the ability of MAb-1 to induce cell death, proliferation arrest and suppression of blastogenesis of PHA-L stimulated T-kmphocytes (CD3+/CD4+), an in vitro cell culture assay, similar to the one described in Example 5, was used.

CD3+ cells were isolated from whole blood, washed and labeled with CSFE using the protcol described in Example 5.

The in vitro cell culture assays were set up in 96 well tissue culture plates. Purified PHA-L were added to specific wells at a concentrations of 1.12 μg/ml. The final cell concentration was 2×10⁵ cells/well. Negative and positive controls were run in triplicates. For PHA-L without anti-HLA-E MAb (control), 10 μl of CFSE labeled cells (2×10⁵ cells in 100 μl/well) were added to 90 μl of PHA-L in AIM-V and 100 μl of AIM-V. For PHA-L with anti-HLA-E MAb-1, 10 μl of CFSE labeled cells (2×10⁵ cells in 100 μl/well) were added to 90 μl of PHA-L in AIM-V and 100 μl AIM-V containing 1/100 dilution of anti-HLA-E MAb-1. One of the three profiles of the controls is shown in FIG. 7.

In FIG. 7, the three upper and lower quadrants of flow cytometric profiles refer to different populations of T-cells (CD3+/CD4+). Two monoclonal antibodies were used: (1) CD3 MAb is indicated by the Y or vertical axis; (2) CD4 MAb is indicated by the X or horizontal axis. Upper left quadrant: CD3 positive cells; lower left quadrant: CD3 negative cells; upper right quadrant: CD3 positive and CD4 positive T-lymphocytes: lower right quadrant: CD3 negative and CD4 negative cells. Cells stained green in the left most quadrants are CD3+/CD4+ primordial naïve T-cells. Cells stained red in the middle quadrants refer to CD3+/CD4+ activated T-cells. The right most quadrants depict CD3+/CD4+ T-Iymphoblasts. Lymphoblasts were identified by the size of the cells, which results in migration of the cells towards left or upper side, indicative of the increased size and possibly granulation.

In comparing PHA w ithout anti-HLA-E MAb- quadrants (upper three) with PHA- with anti-HLA-E MAb-1-quadrants, one notices a decrease in the cell populations of the middle quadrants (red) and the right most quadrants (pink) after adding anti-HLA-E MAb-1. The decrease in number of pink cells (684 to 47) signify the decrease in lymphoblasts in the presence of anti-HLA-E MAb-1 even after exposure to PHA for 70 hrs. Similarly, a decrease in the number of red cells in the middle quadrants (red) (2132 to 409) is indicative of a fall in the number of activated T-lymphocytes. The total number of CD3+/CD4+ T-Iymphocytes decreased from 3356 to 1322 in the presence of anti-HLA-E MAb-1. The loss of red cells in the middle quadrants signify death of CD4+ T-lymphocytes, while loss of pink cells in the right quadrants signify arrest in blastogenesis of CD4+ T-cells.

The results indicate that anti-HLA-E MAb-2 is capable of suppressing T cell proliferation and causing cell death of CD4+ lymphocytes. Both characteristics signify the immunosuppressive nature of anti-HLA-E MAb-1, similar to the immunosuppressive nature of IVIg as seen in Example 6 and anti-HLA-E MAb-2 as seen in Example 8. This experiment was done in triplicate and FIG. 7 is representative of one sample.

A similar experiment was performed to determine the dosimetric effects of anti-HLA-E MAb-1 induced suppression of PHA-stimulated CD4+ T-lymphocytes and T-lymphoblasts. For PHA-L without anti-HLA-E MAb (control), 10 μl of CFSE labeled cells (2×10⁵ cells in 100 μl/well) were added to 90 μl of PHA-L in AIM-V and 100 μl of AIM-V. For PHA-L with anti-HLA-E MAb-1, 10 μl of CFSE labeled cells (2×10⁵ cells in 100 μl/well) were added to 90 μl of PHA-L in AIM-V and 100 μl AIM-V containing anti-HLA-E MAb-1 at 0 dilution, 1/150, 1/100, 1/200, 1/400 and 1/800 dilution. Three values were obtained for each dilution. The mean and standard deviation was determined. FIG. 11 illustrates the percentage change in CD4+ T-Iymphocytes and T-Iymphoblasts at each dilution of MAb-1. As shown in FIG. 11, anti-FILA-E MAb-1 induced suppression of PHA-stimulated CD4+ T-lymphocytes and T-Iymphoblasts in a dose dependent manner.

As shown in FIG. 14, anti-HLA-E MAb-1 and IVIg were both able to inhibit PHA-L stimulated proliferation and blastogenesis of CD4+ T-cells in a similar dose-dependent manner. The differences in the dilutions show the differences in the potency between IVIg and anti-HLA-E Ab. Anti-HLA-E Ab, though functionally similar to IVIg, seems to be more potent than IVIg.

### Example 8

### Anti-HLA-E MAb-2 Induced Cell Death, Proliferation Arrest and Suppression of Blastogenesis of PHA-L Stimulated T-lyvmphocytes (CD3+/CD4+).

This example demonstrates that anti-HLA-E MAb-2 induced cell death, proliferation arrest and suppression of blastogenesis of PHA-L stimulated T-lymphocytes (CD3+/CD4+).

To determine the ability of MAb-2 to induce cell death, proliferation arrest and suppression of blastogenesis of PHA-L stimulated T-lymphoc)tes (CD3+/CD4+), an in vitro cell culture assay, similar to the one described in Example 5, was used.

CD3+ cells were isolated from whole blood, washed and labeled with CSFE using the protcol described in Example 5.

The in vitro cell culture assays were set up in 96 well tissue culture plates. Purified PHA-L were added to specific wells at a concentrations of 1.12 μg/ml. The final cell concentration was 2×10⁵ cells/well. Negative and positive controls were run in triplicates. For PHA-L without anti-HLA-E MAb (control), 10 μl of CFSE labeled cells (2×10⁵ cells in 100 μl/well) were added to 90 μl of PHA-L in AIM-V and 100 μl of AIM-V. For PHA-L with anti-HLA-E MAb-2, 10 μl of CFSE labeled cells (2×10⁵ cells in 100 μl/well) were added to 90 μl of PHA-L in AIM-V and 100 μl AIM-V containing 1/100 dilution of anti-HLA-E MAb-2. One of the three profiles of the controls is presented in FIG. 8.

In FIG. 8, the three upper and lower quadrants of flow cytometric profiles refer to different populations of T-cells (CD3+/CD4+). Two monoclonal antibodies were used: (1) CD3 MAb is indicated by the Y or vertical axis; (2) CD4 MAb is indicated by the X or horizontal axis. Upper left quadrant: CD3 positive cells; lower left quadrant: CD3 negative cells; upper right quadrant: CD3 positive and CD4 positive T-lymphocytes; lower right quadrant: CD3 negative and CD4 negative cells. Cells stained green in the left most quadrants are CD3+/CD4+ primordial naïve T-cells. Cells stained red in the middle quadrants are CD3+/CD4+ activated T-cells. The third right most quadrants refer to CD3+/CD4+ T-lymphoblasts. Lymphoblasts were identified by the size of the cells, which results in migration of the cells towards left or upper side, indicative of the increased size and possibly granulation. In comparing PHA without anti-HLA-E MAb- quadrants (upper three) with PHA-with anti-HLA-E MAb-2-quadrants, one may notice a decrease in the cell populations of the middle quadrants (red) and the right most quadrants (pink) after adding anti-HLA-E MAb-2. The decrease in the number of pink cells (587 to 94) signify the decrease in lymphoblasts in the presence of anti-HLA-E MAb-2 even after exposure to PHA for 70 hrs. Similarly, the decrease in red cells in the middle quadrants (2115 to 566) indicates a decrease in the number of activated T-lymphocytes. The total number of CD3+/CD4+ T-lymphocytes decreased from 3462 to 1489 in the presence of anti-HLA-E MAb-2. The loss of red cells in the middle quadrants signifies death of CD4+ T-cells, while loss of pink cells in the right quadrants signifies arrest in blastogenesis of CD4+ T-cells.

The results indicate that anti-HLA-E MAb-2 is capable of suppressing T cell proliferation and causing cell death of CD4+ lymphocytes. Both characteristics signify the immunosuppressive nature of anti-HLA-E MAb-2, similar to the immunosuppressive nature of IVIg as seen in Example 6. This experiment was done in triplicate and FIG. 8 is representative of one sample.

### Example 9

### IVIg Inhibition of PHA-Induced T Cell Proliferation is Identical to Anti-HLA-E MAb-1 using Carboxyfluorescein Diacetate Succinimidyl Ester (CFSE) Staining Technology

Example 9 demonstrates that IVIg inhibition of PHA-induced T-cell proliferation is similar to anti-HLA-E MAb-1. In this assay system, carboxufluorescein diacetate succinimidyl ester (CFSE) staining technology is used.

Whole blood (20 ml) was drawn from a healthy donor into Acid Citrate Dextrose (ACD) tubes. Fifteen ml of is the blood sample was pipetted into 25 ml of PBS (without Calcium or Magnesium) in a 50-ml conical centrifuge tube and underlayed with Ficoll-Hypaque (10 ml) at RT. After centrifugation (20 min. at 800 g (2000 rpm in H-1000 rotor), 20° C.), the plasma-platelet-containing supernatant was aspirated from above the interface band. The interface band, which that includes the lymphocytes, was then aspirated with <5 ml of fluid and transferred to a new centrifuge tube (50 ml), combining the bands from 2 to 3 Ficoll-Hypaque gradients. PBS was added to the separated bands to a volume of 50 ml and centrifuged (10 min. at 600 g (1500 rpm in H-1000 rotor), 20° C.). The supernatants were aspirated and the pellets in the tubes were combined and resuspended in PBS (10 ml) at RT. PBS was added to a volume of 50 ml and centrifufzed (15 min. 300 g (750 rpm in H-1000 rotor), 20° C.). The resulting lymphocyte pellet was resuspended in PBS (1 ml) at RT and the viable cells were counted. The cells were distributed equally among three Fisher tubes with PBS and centrifuged (1 min. at 1000 g). The supernatant was discarded and the pellet was resuspended and mixed well with 0.8 ml of Lympho-Kwik® T. The mixture was incubated (20 min. at 37° C. or RT) in a water bath or heat block with occasional mix by inverting capped tube. PBS (0.2 ml) was then layered over cell preparation and centrifuged (2 min. at 2000 g). The pellet was resuspended in PBS and centrifuged (1 min. at 1000 g). Washing was repeated once and each pellet was resuspended in 0.8 ml of the following Lympho-Kwik® T Prep. The entire mixing, incubation, centrifugation and resuspension of pellet was repeated. In the final step, the pellet was resuspended in AIM-V medium+1% HEPES at a final concentration of 5×10⁷ cells/ml. An aliquot was tested for purity of T-cells using CD3 monoclonal antibody in flow cytometry.

The cells were labeled with carboxyfluorescein succinimidyl ester (CFSE). CSFE is a fluorescent cell staining dye that is cell permeable and retained for long periods within cells. Within cells, CSFE covalently couples, via its succinimidyl group, to intracellular molecules. Due to this stable linkage, once in a cell, CFSE is not transferred to adjacent cells. The quantity of cells labeled was 10⁵ to 10 cells/ml. Ten percent of heparinized donor plasma was added. Two μl of 5 mM CFSE per milliliter cells (final 10 μM) was added in a tube containing greater than or equal to 6 times the volume of cells. The cells were incubated for 15 min. at RT or for 10 min. at 37° C. The staining was quenched by adding 5 vol ice-cold AIM-V medium (+1% HEPES buffer, with 10% heparinized plasma from donor) and the cells were incubated for 5 min. on ice. The cells were washed three times in the culture medium to ensure that CFSE bound to protein in the supernatant was removed, preventing any subsequent uptake into bystander cells.

The in vitro cell culture assays were set up in 96 well tissue culture plates. Purified PHA-L were added to specific wells at a concentrations of 1.12 The final cell concentration was 2×10⁵ cells/well. Negative and positive controls were run in triplicates. For PHA without IVIg or anti-HLA-E MAb-1 control, 10 μl of CFSE labeled cells (2×10⁵ cells in 100 μl/well) were added to 90 μl of PHA-L in AIM-V and 100 μl of AIM-V. For PHA with IVIg or anti-HLA-E MAb-1 experiments, 10 μl of CFSE labeled cells (2×10⁵ cells in 100 μl/well) were added to 90 μl of PHA-L in AIM-V and 100 μl AIM-V containing different dilutions of dilution of IVIg or anti-HLA-E MAb-1.

FIG. 9A shows the CFSE fluorescence intensity of proliferating T-celss after 70 hours of exposure to PHA. The fluorescence intensity closely follows the predicted sequential halving due to cell division (M1, M2, M3 and M4). FIG. 9B shows the inhibition of PHA-L induced proliferation of CD3+ CFSE+ T-lymphocytes by IVIg at 72 hrs. FIG. 9C shows the inhibition of PHA-L induced proliferation CD3+ CFSE+ T lymphoblasts by IVIg at 72 hrs. FIG. 9D shows the percentage of inhibition of T cell proliferation by IVIg at different dilutions, 72 hrs after PHA-L stimulation. FIG. 9E shows the inhibition of PHA-L induced proliferation CD3+ CFSE+ T lymphocytes by anti-HLA-E MAb-1 at 72 hrs. FIG. 9F shows the inhibition of PHA-L induced proliferation CD3+ CFSE+ T lymphoblasts by anti-HLA-E MAb-1 at 72 hrs. FIG. 9G shows the percentage inhibition of T cell proliferation by anti-HLA-E MAb-1 at different dilutions, 72 hrs after PHA-stimulation.

### Example 10

### Analysis of the Dosimetric Effects of IVIg on PHA-L Stimulated CD3+/CD8+ Blastogenesis and Proliferation of CD8+ T-lymphoblasts

This example provides a dosimetric analysis of the effects of IVIg on PHA-L stimulated CD3+/CD8+ blastogenesis and proliferation of CD8+ T-Iymphoblasts.

To determine the dosimetric effects of IVIg on PHA-L stimulated CD3+/CD8+ blastogenesis and proliferation of CD8+ T-lymphoblasts, an in vitro cell culture assay, similar to the one described in Example 5, was used.

CD3+ cells were isolated from whole blood, washed and labeled with CSFE using the protcol described in Example 5.

The in vitro cell culture assays were set up in 96 well tissue culture plates. Purified PHA-L were added to specific wells at a concentrations of 1.12 μg/ml. The final cell concentration was 2×10⁵ cells/well. For PHA-L without IVIg control, 10 μl of CFSE labeled cells (2×10⁵ cells in 100 μl/well) were added to 90 μl of PHA-L in AIM-V and 100 μl of AIM-V. For PHA-L with IVIg, 10 μl of CFSE labeled cells (2×10⁵ cells in 100 μl/well) were added to 90 μl of PHA-L in AIM-V and 100 μl AIM-V containing IVIg at 0 dilution, 1/10, 1/20, 1/40, 1/80 and 1/160 dilutions. Three values were obtained for each dilution. The mean and standard deviation was determined.

Cells were stained with two monoclonal antibodies: (1) CD3 MAb as indicated by the Y or vertical axis flow cytometric profile as in FIGS. 6; and (2) CD8 MAb as indicated by the X or horizontal axis in the flow cytometric profile (data not shown).

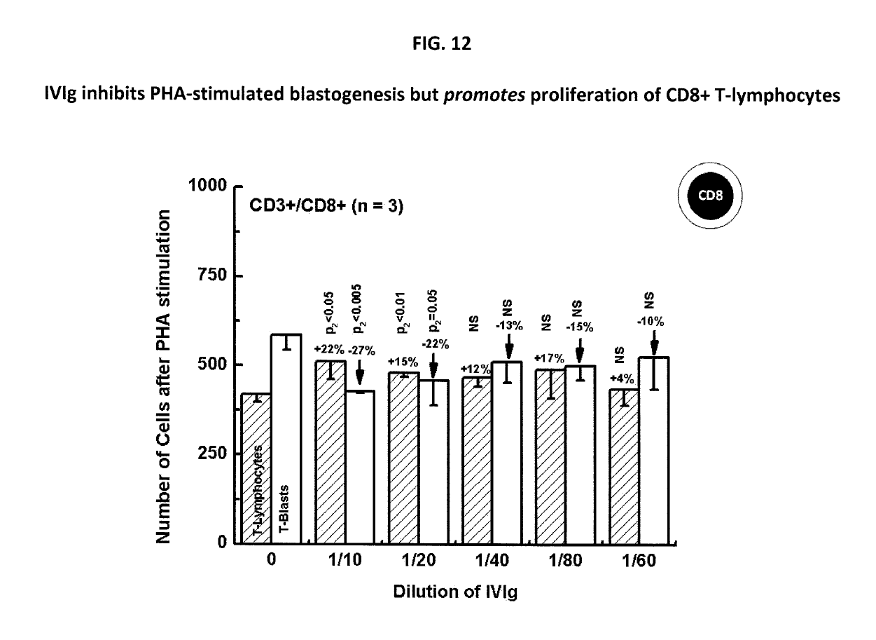

FIG. 12 illustrates the percentage change in CD3+/CD8+ T-lymphocytes and CD8+ T-lymphoblasts at each dilution of IVIg. As shown in FIG. 12, IVIg at different dilutions induced suppression of PHA-stimulated CD3+/CD8+ blastogenesis but promote the proliferation of CD8+ T-lymphoblasts.

### Example 11

### Analysis of the Dosimetric Effects of Anti-HLA E MAb (MAb-1) on PHA-L Stimulated CD3+/CD8+ Blastogenesis and Proliferation of CD8+ T-lymphoblasts

This example provides a dosimetric analysis of the effects of MAb-1 on PHA-L stimulated CD3+/CD8+ blastogenesis and proliferation of CD8+ T-Iymphoblasts.

To determine the dosimetric effects of IVIg on PHA-L stimulated CD3+/CD8+ blastogenesis and proliferation of CD8+ T-Iymphoblasts, an in vitro cell culture assay, similar to the one described in Example 5, was used.

CD3+ cells were isolated from whole blood, washed and labeled with CSFE using the protcol described in Example 5.

The in vitro cell culture assays were set up in 96 well tissue culture plates. Purified PHA-L were added to specific wells at a concentrations of 1.12 μg/ml. The final cell concentration was 2×10⁵ cells/vvell. Negative and positive controls were run in triplicates. For PHA-L without anti-HLA-E MAb control, 10 μl of CFSE labeled cells (2×10⁵ cells in 100 μl/well) were added to 90 μl of PHA-L in AIM-V and 100 μl of AIM-V. For PHA-L with anti-HLA-E MAb, 10 μl of CFSE labeled cells (2×10⁵ cells in 100 μl/well) were added to 90 μl of PHA-L in AIM-V and 100 μl AIM-V containing anti-HLA-E MAb-1 at 0 dilution, 1/10, 1/20. 1/40, 1/80 and 1/160 dilutions. Three values were obtained for each dilution. The mean and standard deviation was determined.

Two monoclonal antibodies were used: (1) CD3 MAb as indicated by the Y or vertical axis flow cytometric profile as in FIGS. 6; and (2) CD8 MAb as indicated by the X or horizontal axis in the flow cytometric profile (data not shown).

As shown in FIG. 13, MAb-1 at different dilutions induced suppression of PHA-stimulated CD3+/CD8+ blastogenesis but promotion of proliferation of CD8+ T-lymphoblasts.

As shown in FIG. 14, both MAb-1 and IVIg were unable to inhibit proliferation of PHA-L stimulated CD8+ T-cells.

### Example 12

### Analysis of the Presence of Soluble HLA-E Heavy Chains in the Sera of Kidney and Liver Allograft Recipients using Gel Electrophoresis and Western Blot

This example provides an analysis of the presence of soluble HLA-E heavy chains in the sera of kidney and liver transplant recipients.

To evaluate the presence of HLA-E heavy chains in the sera of kidney and liver transplant recipients, sera from kidney and liver transplant recipients were aliquoted into 8 μl samples and each sample was run under reducing conditions in separate wells of a 12% polyacrylamide gel. The gels were subject to Western blotting. Western blots were immunostained with murine MAb MEM-E/02 and −E/06 separately. HLA I heavy chains range in molecular weight from 47 to 32 kDa, β2-microglobulin has a molecular weight of 12 kDa.

FIG. 15 depicts the presence of soluble HLA-E in the sera of kidney transplant patients. The presence of HLA-E heavy chains in the sera of kidney transplant recipients (TFL-Michigan Sera: Patient ID: 1, Mi-9707, 2, Mi-11151, 3, Mi-11553, 4. Mi-10788, 5, Mi-11909, 6, Mi-12172, 7, Mi-13041, 8, Mi-13100) was detected through Western blot and immunostaining with two anti-HLA-E antibodies that bind to heavy chain, MAb MEM-E/02 (A) and MAb MEM-E/06(B).

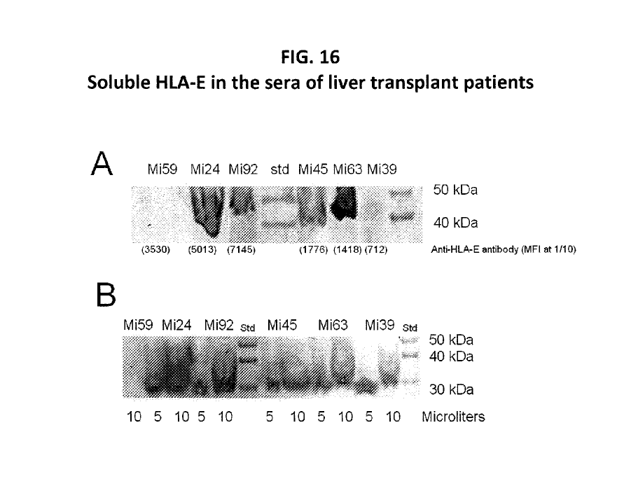

FIG. 16 depicts Western blots showing the presence of soluble HLA-E in the sera of liver transplant recipients. Electropherograms were obtained without (16A) and with (16B) reducing agents. The presence of HLA-E heavy chains in the sera of liver transplant patients (TEL-Milan Sera(Patient ID: Mi-59, Mi-24, Mi-92, Mi-45, Mi-63, Mi-39)) was detected through Western blot and immunostaining with murine MAb MEM-E/02. Upper row labeling shows patient ID and the lower row labeling shows MEI of sera at 1/10 dilution. The molecular weight of b2-m, which is not shown in the Western blots, is about 12 kDa. In some sera only one heavy chain fraction was observed, while in other sera, three fractions were observed.

Table 4 demonstrates that soluble HLA-E in the sera of liver allograft recipients (Mi127, Mi114. Mi92 & Mi59; sera diluted 1/100) was able to inhibit HLA Ia reactivity of the murine monoclonal antibody (MAb) MEM-E/02. Inhibition is expressed as percentage inhibition of Mean Fluorescent Intensity (MEI) of the MEM-E/02.

Table 5 demonstrates that different dilutions of soluble HLA-E in the IgG-free serum of a liver allograft recipient (Mi 92) inhibited HLA-la reactivity of the murine monoclonal antibody (MAb) MEM-E/02. The inhibition is compared with that of HLA-E. The values are expressed as Mean Fluorescent Intensity (MEI) of the MAb. For preparing IgG-free serum, the patient's serum was passed through a Protein G column.

The analysis in FIGS. 15A and 15B demonstrate that only HLA-E heavy chains are stained. In some sera only one faction was observed while, in others, 3 fractions were observed. Together, the evidence in FIGS. 15A and 15B demonstrate the presence of soluble HLA I in circulation.

Tables 4 and 5 further demonstrate that the soluble HLA-E binds to murine monoclonal antibodies MAb MEM-E/02 and the tables together with the figures enable one to infer that this binding is to the HLA-E heavy chain. In circulation. anti-HLA-E antibodies can bind, block and neutralize HLA I heavy chains.

TABLE 4

| | | MFI and Percentage Inhibition by MAb MEM-E/02 at dilution 1/600 | | | | | | | |
|---|---|---|---|---|---|---|---|---|---|
| | | Serum ID of liver second allograft recipients | | | | | | | |
| HLA-Ia | MEM-E/02 | Mi127 | | Mi114 | | Mi92 | | Mi59 | |
| alleles | Reactivity | | % | | % | | % | | % |
| Tested | Rank | MFI | inhibition | MFI | inhibition | MFI | inhibition | MFI | inhibition |
| Neg | | 35 | | 107 | | 123 | | 248 | |
| A*2402(A24) | 62 | 1425 | 34 | <500 | | <500 | | <500 | |
| A*2403(A24) | 63 | 1327 | 36 | <500 | | <500 | | <500 | |
| A*2501(A25) | >65 | 1223 | 36 | <500 | | 722 | 9 | <500 | |
| A*2601(A26) | 60 | 2659 | 15 | <500 | | <500 | | <500 | |
| A*2901(A29) | 48 | <500 | | <500 | | 678 | 38 | <500 | |
| A*3002(A30) | >65 | <500 | | 803 | 100 | <500 | | <500 | |
| A*3201(A32) | >65 | <500 | | 742 | 79 | <500 | | <500 | |
| A*3301(A33) | 50 | 3170 | 18 | <500 | | 522 | | <500 | |
| B*0801(B8) | >65 | <500 | | <500 | | 990 | 50 | <500 | |
| B*1301(B13) | 5 | <500 | | <500 | | 978 | 25 | <500 | |
| B*1401(B64) | 7 | 2990 | 13 | <500 | | 701 | 7 | <500 | |
| B*1511(B75) | 11 | 1637 | 18 | <500 | | 946 | | <500 | |
| B*1513(B77) | 41 | <500 | | 1546 | 56 | <500 | | <500 | |
| B*1516(B63) | 43 | 1836 | 15 | <500 | | 581 | | <500 | |
| B*1801(B18) | 36 | 1992 | 17 | <500 | | <500 | | <500 | |
| B*2708(B27) | >65 | 2427 | 11 | 2255 | 29 | <500 | | <500 | |
| B*3801(B38) | >65 | 2504 | 24 | 929 | 76 | <500 | | <500 | |
| B*3901(B39) | 14 | 1480 | 16 | 530 | | 737 | 60 | <500 | |
| B*4403(B44) | >65 | 1905 | 11 | <500 | | 600 | | <500 | |
| B*4501(B45) | 9 | 1726 | 11 | <500 | | <500 | | <500 | |
| B*4801(B48) | 12 | 1784 | 16 | 969 | 22 | <500 | | <500 | |
| B*4901(B49) | 38 | <500 | | <500 | | 890 | | <500 | |
| B*5001(B50) | >65 | 1345 | 18 | <500 | | 1418 | 45 | <500 | |
| B*5102(B51) | 39 | 2518 | 15 | 507 | | <500 | | <500 | |
| B*5201(B52) | 10 | 3075 | 14 | 678 | 88 | <500 | | <500 | |
| B*5301(B53) | 15 | 2019 | 17 | <500 | | 797 | | <500 | |
| B*5401(B54) | 31 | 3094 | 12 | 990 | 95 | <500 | | <500 | |
| B*5703(B57) | 41 | 917 | 11 | 1345 | 10 | <500 | | <500 | |
| B*5801(B58) | 18 | 1369 | 19 | 2110 | 37 | <500 | | <500 | |
| B*5901(B59) | 20 | 848 | 8 | 930 | 3 | <500 | | <500 | |
| B*6701(B67) | >65 | 2396 | 13 | <500 | | <500 | | <500 | |
| CW*0304) | 44 | <500 | | <500 | | 1025 | 38 | 557 | |
| CW*0401(Cw4) | >65 | <500 | | <500 | | 826 | 60 | 4591 | 22 |
| CW*0801(Cw8) | 8 | <500 | | <500 | | 1201 | 45 | <500 | |
| CW*1203(Cw12) | 36 | <500 | | <500 | | 788 | 65 | <500 | |
| CW*1502(Cw15) | >65 | <500 | | <500 | | 1182 | | 2304 | 24 |
| CW*1701(Cw17) | 28 | <500 | | <500 | | 1056 | 4 | <500 | |
| CW*1802(Cw18) | 1 | <500 | | <500 | | 1442 | 46 | 1085 | 27 |

TABLE 5

| | Inhibition of HLA-Ia reactivity of MAb MEM-E/02 by rHLA-E & | | | | | | | | |
|---|---|---|---|---|---|---|---|---|---|
| | IgG-free serum* of a liver second allograft recipient | | | | | | | | |
| MEM-E/02 | MFI of MAb MEM-E/02 | | | MFI of MAb MEM-E/02 | | | MFI of MAb MEM-E/02 | | |
| positive | 1/100 | | | 1/200 | | | 1/400 | | |
| HLA-Ia | Untreated | incubated with | | Untreated | incubated with | | Untreated | incubated with | |
| alleles | PBS only | Serum | rHLA-E | PBS only | Serum | rHLA-E | PBS only | Serum | rHLA-E |
| B*0801 | 2035 | 1440 | 219 | 1051 | 869 | 152 | 684 | 520 | 148 |
| B*1401 | 7341 | 7080 | 230 | 5326 | 5372 | 163 | 3865 | 4091 | 142 |
| B*1402 | 2523 | 2386 | 115 | 1758 | 1690 | 108 | 1205 | 1173 | 88 |
| B*1502 | 2699 | 2913 | 169 | 1835 | 2009 | 146 | 1204 | 1367 | 149 |
| B*1511 | 3431 | 3318 | 192 | 2359 | 2418 | 125 | 1653 | 1715 | 111 |
| B*1513 | 2649 | 2699 | 256 | 1903 | 2010 | 241 | 1395 | 1381 | 228 |
| B*1801 | 3545 | 3514 | 154 | 2580 | 2602 | 149 | 1830 | 1865 | 127 |
| B*2705 | 1161 | 1096 | 101 | 842 | 754 | 72 | 551 | 481 | 64 |
| B*2708 | 1936 | 1749 | 191 | 1409 | 1250 | 147 | 991 | 870 | 122 |
| B*3701 | 4784 | 4253 | 80 | 3133 | 2721 | 43 | 2114 | 1791 | 39 |
| B*4001 | 2838 | 2676 | 235 | 2152 | 1939 | 90 | 1575 | 1384 | 87 |
| B*4002 | 2900 | 2995 | 174 | 2058 | 2194 | 194 | 1412 | 1450 | 177 |
| B*4006 | 8593 | 9087 | 175 | 7009 | 7410 | 105 | 5288 | 5553 | 60 |
| B*4101 | 3796 | 3390 | 145 | 2844 | 2433 | 82 | 2054 | 1752 | 84 |
| B*4402 | 2719 | 2372 | 123 | 2027 | 1645 | 66 | 1458 | 1139 | 44 |
| B*4403 | 3678 | 3895 | 89 | 2785 | 2874 | 75 | 1929 | 2007 | 48 |
| B*4501 | 4772 | 4410 | 75 | 3672 | 3288 | 58 | 2686 | 2265 | 51 |
| B*4601 | 2521 | 2278 | 123 | 1758 | 1676 | 103 | 1151 | 1119 | 103 |
| B*4701 | 3646 | 3015 | 145 | 2885 | 2344 | 67 | 2152 | 1585 | 54 |
| B*4801 | 3054 | 2682 | 224 | 2121 | 1934 | 169 | 1553 | 1326 | 166 |
| B*5101 | 2206 | 2309 | 150 | 1536 | 1591 | 134 | 1038 | 1125 | 128 |
| B*5201 | 3137 | 3031 | 155 | 2313 | 2020 | 87 | 1683 | 1399 | 70 |
| B*5801 | 3987 | 4571 | 160 | 2831 | 3421 | 70 | 1854 | 2421 | 48 |
| B*8201 | 2469 | 2416 | 132 | 1792 | 1760 | 151 | 1197 | 1203 | 140 |
| CW*0202 | 3876 | 4113 | 220 | 3015 | 2949 | 107 | 2256 | 2156 | 79 |
| CW*0302 | 2451 | 2199 | 188 | 1826 | 1631 | 137 | 1362 | 1183 | 130 |
| CW*0303 | 3196 | 2877 | 139 | 2362 | 2090 | 64 | 1648 | 1494 | 50 |
| CW*0304 | 2846 | 2703 | 181 | 2170 | 2029 | 99 | 1572 | 1481 | 75 |
| CW*0501 | 7178 | 7529 | 201 | 5831 | 6259 | 147 | 4544 | 4627 | 87 |
| CW*0602 | 4041 | 4184 | 88 | 2673 | 2188 | 99 | 1716 | 1562 | 52 |
| CW*0702 | 3076 | 3093 | 220 | 1842 | 1539 | 137 | 1320 | 1066 | 120 |
| CW*0801 | 5888 | 6071 | 163 | 4502 | 4746 | 104 | 3340 | 3528 | 102 |
| CW*1402 | 3474 | 3286 | 165 | 2590 | 2412 | 79 | 1901 | 1742 | 60 |
| CW*1502 | 2695 | 2755 | 170 | 1890 | 1859 | 86 | 1414 | 1270 | 79 |
| CW*1601 | 2736 | 2609 | 188 | 2001 | 1826 | 112 | 1468 | 1245 | 87 |
| CW*1701 | 3356 | 2817 | 376 | 2267 | 1931 | 237 | 1671 | 1357 | 184 |
| CW*1802 | 10295 | 10606 | 122 | 8827 | 8806 | 56 | 7037 | 7225 | 60 |
| p (2-tail) | | 0.0913 | <0.0001 | | 0.0413 | <0.0001 | | 0.0385 | <0.0001 |

| | Inhibition of HLA-Ia reactivity of MAb MEM-E/02 by rHLA-E & | | | | | |
|---|---|---|---|---|---|---|
| | IgG-free serum* of a liver second allograft recipient | | | | | |
| MEM-E/02 | MFI of MAb MEM-E/02 | | | f MAb MEM-E/02 | | |
| positive | 1/800 | | | 1/1600 | | |
| HLA-Ia | Untreated | incubated with | | Untreated | incubated with | |
| alleles | PBS only | Serum | rHLA-E | PBS only | Serum | rHLA-E |
| B*0801 | 306 | 262 | 141 | 262 | 194 | 128 |
| B*1401 | 1999 | 1917 | 134 | 1407 | 1034 | 134 |
| B*1402 | 576 | 517 | 95 | 408 | 301 | 97 |
| B*1502 | 611 | 603 | 162 | 426 | 350 | 155 |
| B*1511 | 796 | 716 | 97 | 529 | 388 | 96 |
| B*1513 | 760 | 686 | 242 | 582 | 458 | 246 |
| B*1801 | 899 | 777 | 118 | 628 | 441 | 109 |
| B*2705 | 270 | 215 | 61 | 191 | 132 | 59 |
| B*2708 | 521 | 417 | 131 | 396 | 272 | 134 |
| B*3701 | 993 | 784 | 29 | 636 | 376 | 19 |
| B*4001 | 804 | 589 | 85 | 560 | 324 | 83 |
| B*4002 | 703 | 655 | 194 | 515 | 403 | 196 |
| B*4006 | 2839 | 2586 | 51 | 1872 | 1335 | 49 |
| B*4101 | 960 | 720 | 68 | 645 | 392 | 70 |
| B*4402 | 669 | 470 | 48 | 447 | 248 | 51 |
| B*4403 | 983 | 850 | 44 | 615 | 441 | 43 |
| B*4501 | 1297 | 897 | 52 | 808 | 464 | 45 |
| B*4601 | 543 | 475 | 87 | 375 | 274 | 85 |
| B*4701 | 1168 | 737 | 57 | 804 | 389 | 55 |
| B*4801 | 837 | 640 | 172 | 598 | 412 | 166 |
| B*5101 | 515 | 483 | 99 | 347 | 281 | 99 |
| B*5201 | 786 | 609 | 76 | 537 | 337 | 72 |
| B*5801 | 835 | 938 | 45 | 524 | 485 | 48 |
| B*8201 | 614 | 522 | 139 | 417 | 313 | 136 |
| CW*0202 | 1149 | 1060 | 76 | 785 | 578 | 73 |
| CW*0302 | 681 | 558 | 121 | 478 | 335 | 115 |
| CW*0303 | 805 | 634 | 46 | 530 | 332 | 33 |
| CW*0304 | 783 | 667 | 73 | 512 | 370 | 64 |
| CW*0501 | 2450 | 2215 | 70 | 1618 | 1262 | 68 |
| CW*0602 | 839 | 694 | 54 | 581 | 366 | 44 |
| CW*0702 | 663 | 512 | 99 | 473 | 295 | 88 |
| CW*0801 | 1755 | 1648 | 109 | 1104 | 865 | 103 |
| CW*1402 | 1006 | 803 | 48 | 653 | 432 | 48 |
| CW*1502 | 698 | 581 | 80 | 485 | 317 | 80 |
| CW*1601 | 752 | 582 | 87 | 524 | 338 | 83 |
| CW*1701 | 847 | 676 | 161 | 619 | 392 | 155 |
| CW*1802 | 4089 | 3814 | 35 | 2751 | 2067 | 30 |
| p (2-tail) | | <0.0001 | <0.0001 | | <0.0001 | <0.0001 |

*IgG-free serum is obtained after passing the serum through Protein-G column; IgG anti-idiotypic antibodies, present if any, are removed

1. A pharmaceutical composition comprising purified antibodies in a pharmaceutically acceptable carrier, wherein said purified antibodies are chimeric, humanized or human anti-HLA-E antibodies immunoreactive to the polypeptide heavy chain of HLA-E and are not immunoreactive to the polypeptide heavy chain of HLA-F or HLA-G or to β2-microglobulin.

2. The pharmaceutical composition of claim 1, wherein said anti HLA E antibodies are immunoreactive to the polypeptide heavy chains of one or more HLA-A alleles and a plurality of HLA-B and HLA-Cvv alleles.

3. The pharmaceutical composition of claim 1 wherein each said polypeptide heavy chain is free, or associated with β2-microglobulin or associated with another polypeptide heavy chain of the same alleles, and wherein each polypeptide heavy chain is expressed on a cell surface or present in circulation or a body fluid.

4. The pharmaceutical composition of any of claim 1 wherein the immunoreactivity of said anti HLA-E antibodies is blocked by the peptide sequences QFAYDGKDY (SEQ ID NO: 5) and DTAAQI (SEQ ID NO: 8).

5. The pharmaceutical composition of claim 1 wherein the immunoreactivity of said anti HLA-E antibodies is blocked by any peptide according to one of the sequences QFAYDGKDY (SEQ ID NO: 5), LNEDLRSWTA (SEQ ID NO: 7) and DTAAQI (SEQ ID NO: 8).

6. The pharmaceutical composition of claim 1 wherein the immunoreactivity of said anti HLA-E antibodies is blocked by a peptide according to the sequence EYWDRETR (SEQ ID NO: 2).

7. The pharmaceutical composition of claim 1 wherein the immunoreactivity of said and HLA-E antibodies is blocked by a peptide according to the sequence EPPKTHVT (SEQ ID NO: 12).

8. The pharmaceutical composition of claim 1 wherein the immunoreactivity of said anti HLA-E antibodies is blocked by a peptide according to the sequence RAY LED (SEQ ID NO: 10).

9. The pharmaceutical composition of any of claim 1 wherein the immunoreactivity of said anti HLA-E antibodies is blocked by a peptide according to the sequence ⁶⁵RSARDTA⁷¹ (SEQ ID NO: 13).

10. The pharmaceutical composition of claim 1 wherein the immunoreactivity of said anti HLA-E antibodies is blocked by a peptide according to the sequence ¹⁴³SEQKSNDASE¹⁵² (SEQ ID NO: 14).

11. The pharmaceutical composition of claim 1 wherein said anti HLA-E antibodies are purified monoclonal antibodies, purified polyclonal antibodies, recombinantly produced antibodies, Fab fragments, F(ab′) fragments or epitope-binding fragments.

12. The pharmaceutical composition of claim 1 wherein the composition is suitable for subcutaneous, intravenous, or intramuscular administrations.

13. The pharmaceutical composition of claim 1 wherein the composition is capable of modulating naïve and/or activated CD3+/CD4+ T-cells in a recipient of the pharmaceutical composition.

14. The pharmaceutical composition of claim 1 wherein the composition is capable of immunomodulating naïve and/or activated CD3+/CD8+ T-cells in a recipient of the pharmaceutical composition.

15. The pharmaceutical composition of claim 1 wherein the composition is capable of inducing cell death of naïve and/or activated CD3+/CD4+ T-cells in a recipient of the pharmaceutical composition.

16. The pharmaceutical composition of claim 1 wherein the composition is capable of suppressing formation of T-cell dependent HLA antibodies in a recipient.

17. The pharmaceutical composition of claim 1 wherein the composition is capable of blocking or neutralizing a proinflammatory or adverse effect of said polypeptide heavy chain by interfering with the binding of said polypeptide heavy chain to a lymphocyte bound receptor, wherein said polypeptide is soluble and in circulation or a body fluid.

18. The pharmaceutical composition of claim 1 wherein the composition is capable of clearing one or more of said HLA-E, HLA-A, HLA-B or HLA-Cw polypeptide heavy chain from circulation or a body fluid, wherein said polypeptide heavy chain is soluble.

19. The pharmaceutical composition of claim 1 wherein said anti HLA E antibodies are immunoreactive to HLA Ia antigens similar to therapeutically administered commercial preparations of Intravenous immunoglobulin (IVIg).

20. The pharmaceutical composition of claim 1 wherein said anti-HLA-E antibodies have immunomodulatory activity comparable to Intravenous immunoglobulin (IVIg).

21. The pharmaceutical composition of claim 1 wherein the composition is therapeutically effective for the treatment of one or more inflammatory diseases treatable by a therapeutically administered commercial preparation of Intravenous immunoglobulin (IVIg).

22. The pharmaceutical composition of any one of claim 1 wherein said anti HLA-E antibodies are purified IgG antibodies.

23. The pharmaceutical composition of any one of claim 1 wvherein said anti HLA-E antibodies are purified IgG1 antibodies.

24. A method of preventing, managing, treating and/or ameliorating graft rejection, the method comprising administering to a human an effective amount of the pharmaceutical composition of claim 1.

25. The method of claim 24, wherein the graft is a tissue graft.

26. The method of claim 24, wherein the graft is an organ graft.

27. The method of claim 24, wherein the organ graft is a heart, kidney or liver graft.

28. The method of claim 24, wherein the graft is a cell graft.

29. The method of claim 24, wherein the cell graft is bone marrow transplantation or a blood transfusion.

30. A method of managing, treating and/or ameliorating an inflammatory condition selected from the group consisting of: a hematological disease, nephropathy, neuropathy, a bacterial infection, a viral infection, an autoimmune disease, cardiomyopathy, an eye or ear disease, a lung disease, recurring pregnancy loss, Behget syndrome, chronic fatigue syndrome, congenital heart block, diabetes mellitus, acute idiopathic dysautonomia, opsoclonus-myoclonus, Rasmussen syndrome, Reiter syndrome, or Vogt-Koyanagi-Harada syndrome, the method comprising administering to a mammal an effective amount of the pharmaceutical composition of claim 1.

31. The method of claim 24 wherein at least 99% of said antibodies are purified anti-HLA-E antibodies.


SEQUENCE LISTING (14 sequences)

SEQ ID NO: 1 - protein, 9 aa, Artificial Sequence
  Synthetic peptide
PRAPWMEQE

SEQ ID NO: 2 - protein, 8 aa, Artificial Sequence
  Synthetic peptide
EYWDRETR

SEQ ID NO: 3 - protein, 8 aa, Artificial Sequence
  Synthetic peptide
AGSHTLQW

SEQ ID NO: 4 - protein, 7 aa, Artificial Sequence
  Synthetic peptide
RFLRGYE

SEQ ID NO: 5 - protein, 9 aa, Artificial Sequence
  Synthetic peptide
QFAYDGKDY

SEQ ID NO: 6 - protein, 7 aa, Artificial Sequence
  Synthetic peptide
AYDGKDY

SEQ ID NO: 7 - protein, 10 aa, Artificial Sequence
  Synthetic peptide
LNEDLRSWTA

SEQ ID NO: 8 - protein, 6 aa, Artificial Sequence
  Synthetic peptide
DTAAQI

SEQ ID NO: 9 - protein, 7 aa, Artificial Sequence
  Synthetic peptide
DTAAQIS

SEQ ID NO: 10 - protein, 6 aa, Artificial Sequence
  Synthetic peptide
RAYLED

SEQ ID NO: 11 - protein, 6 aa, Artificial Sequence
  Synthetic peptide
TCVEWL

SEQ ID NO: 12 - protein, 8 aa, Artificial Sequence
  Synthetic peptide
EPPKTHVT

SEQ ID NO: 13 - protein, 7 aa, Artificial Sequence
  Synthetic peptide
RSARDTA

SEQ ID NO: 14 - protein, 10 aa, Artificial Sequence
  Synthetic peptide
SEQKSNDASE